SEQUENCE LISTING (9 sequences)

SEQ ID NO: 1 - protein, 9 aa, Artificial Sequence
  Synthetic
GSYWYDVWF

SEQ ID NO: 2 - protein, 13 aa, Artificial Sequence
  Synthetic
  Disulfid (2)..(12)
  SITE (5)..(5): X is any amino acid
DCAWXLGELVWCT

SEQ ID NO: 3 - protein, 56 aa, Artificial Sequence
  Synthetic
  acetylation (1)..(1): N-terminal
  non_ter (56)..(56)
DKEWILQKIYEIMRLLDELGHAEASMRVSDLIYEFMKKGDERLLEEAERLLEEVER

SEQ ID NO: 4 - protein, 13 aa, Artificial Sequence
  Synthetic
  acetylation (1)..(1)
  disulfid (2)..(12)
DCAWNLGELVWCT

SEQ ID NO: 5 - protein, 13 aa, artificial sequence
  synthetic
  acetylation (1)..(1)
  disulfid (2)..(12)
  non_ter (13)..(13)
DCAWHLGELVWCT

SEQ ID NO: 6 - protein, 13 aa, Artificial Sequence
  Synthetic
  non_ter (1)..(1)
  disulfid (2)..(12)
DCAWHLGELVWCT

SEQ ID NO: 7 - protein, 14 aa, Artificial Sequence
  Synthetic
  SITE (11)..(11): X is Tic
  SITE (12)..(12): X is Aib
  Non_term (14)..(14)
ASYHLGELVWXXCE

SEQ ID NO: 8 - protein, 13 aa, Artificial Sequence
  Synthetic
  disulfid (2)..(12)
DCAWHLGELVWCT

SEQ ID NO: 9 - protein, 15 aa, Artificial Sequence
  Synthetic
  disulfid (1)..(15)
  disulfid (3)..(13)
CDCAWHLGELVWCTC

(19) United States
(12) Patent Application Publication
(10) Pub. No.: US 2024/0252674 A1
(43) Pub. Date: Aug. 1, 2024

(54) AGENTS FOR DIRECTED CONJUGATION TECHNIQUES AND CONJUGATED PRODUCTS

(71) Applicant: Biohaven Therapeutics Ltd., New Haven, CT (US)

(72) Inventors: Wieslaw KAZMIERSKI, Raleigh, NC (US); Gene M. DUBOWCHIK, Middlefield, CT (US); Reese M. CALDWELL, Cambridge, MA (US); David Adam SPIEGEL, Hamden, CT (US)

(21) Appl. No.: 18/555,857

(22) PCT Filed: May 17, 2022
(86) PCT No.: PCT/US2022/029535

§ 371 Date: Oct. 17, 2023

Related U.S. Application Data
(60) Provisional application No. 63/189,522, filed on May 17, 2021.

(51) Int. Cl. A61K 47/68; A61K 47/64

(52) U.S. Cl. CPC A61K 47/6889; A61K 47/64; A61K 47/6835

(57) ABSTRACT

Among other things, the present disclosure provides technologies for site-directed conjugation of various moieties of interest to target agents. In some embodiments, the present disclosure utilizes target binding moieties to provide high conjugation efficiency and selectivity. In some embodiments, provided technologies are useful for preparing antibody conjugates.

### CROSS REFERENCE TO RELATED APPLICATION

This application claims priority of U.S. Provisional Appl. No. 63/189,522 filed May 17, 2021, which is hereby incorporated by reference in its entirety.

### FIELD OF THE INVENTION

The present invention relates to conjugated therapy enhancers that are useful for preventing and/or treating various conditions, disorders, or diseases. Specifically, the present invention relates to protein conjugates such as antibody-drug conjugates that are capable of acting as therapy enhancers.

### BACKGROUND

Conjugated therapy enhancers have been extensively used for preventing and/or treating various conditions, disorders, and diseases. Such enhancers typically include a therapeutically active molecule, such as an antibody, linked to a moiety having affinity to a particular target implicated in the condition, disorder, or disease. However, the majority of known conjugation techniques are not directed to a specific site of the therapeutically active molecule, and usually result in a mixture of conjugates. There remains a need in the development of site-specific conjugation techniques that provide reaction products with high degree of homogeneity.

### SUMMARY

The present disclosure is directed to compositions that include therapy enhancer agents containing moieties of interest conjugated to target agent moieties at specific locations.

In an embodiment, provided is a compound having the structure of formula R-I:

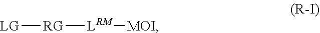

or a salt thereof, wherein:

LG is a group comprising a target binding moiety that binds to a target agent,
RG is a reactive group of formula -L^{LG2}-L^{LG3}-L^{LG4}-L^{RG1}-L^{RG2}-, wherein
L^{LG2} is —NH—C(O)O—C(R′)₂—, wherein each R′ is independently H or C1-C10 alkyl, wherein R′ are optionally connected to form a ring;
L^{LG3} is an optionally substituted aryl ring;
L^{LG4-} NH— or —O—;
L^{RG1} is —C(O)—, —S(O)—, —OS(O)₂—, or —OP(O)(OR)₂—; and
LRG2- is a covalent bond or [—C(R″)₂C(R″)═C(R″)]C(O)—, wherein each R″ is independently
H or C1-C10 alkyl, wherein any two R″ are optionally connected to form a ring;
   L^{RM} is a linker; and
   MOI is a moiety of interest.

In another embodiment, provided is a composition including the above compound.

### BRIEF DESCRIPTION OF THE DRAWINGS

These and/or other aspects will become apparent and more readily appreciated from the following description of the embodiments, taken in conjunction with the accompanying drawings in which:

FIG. 1. Target binders and a target conjugation process (top) and a schematic view of target binder interacting with Lys 246 of an immunoglobulin target.

FIG. 2. Binding specificity data for a linear peptide IgG binder. Data for the GSYWYDVWF peptide (SEQ ID NO:1) is shown.

FIG. 3. Binding specificity data for a cyclic peptide IgG binder. Data for the DCAWXLGELVWCT (SEQ ID NO:2) peptide is shown.

FIG. 4. 4A shows a target conjugation where the reactive compound has a reactive group that is an aza-Michael acceptor, 4B shows a target conjugation where the reactive compound has a reactive group that releases CO2 upon conjugation.

FIG. 5. A target binder, including the peptide DKEWILQKIYEIMRLLDELGHAEASMRVSDLIYEFMKKGDERLLEEAERLLEEVER (SEQ ID NO:3)

FIG. 6. Exemplified target binding groups, according to some embodiments. Ac-DCAWNLGELVWCT (SEQ ID NO:4), Ac-DCAWHLGELVWCT-R (SEQ ID NO:5), R-DCAWHLGELVWCT (SEQ ID NO:6), ASYHLGELVW-Tic-Aib-CE-R (SEQ ID NO:7)

FIGS. 7 and 8. Synthesis of exemplified target binding groups.

FIG. 9. Exemplified LG-RG groups.

### DETAILED DESCRIPTION

The following detailed description is provided to aid those skilled in the art of this disclosure. Exemplified embodiments will hereinafter be described in detail. However, these embodiments are only examples, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in this disclosure.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C₁-C₁₀ amine group, a nitro group (—NO₂), a C₁-C₁₀ alkyl group, a C₃-C₁₀ cycloalkyl group, a C₆-C₁₂ aryl group, a C₁-C₁₀ alkoxy group, a C₁-C₁₀ trifluoroalkyl group such as a trifluoromethyl group (—CF₃) and the like, or a cyano group (—CN) instead of at least one hydrogen of a substituting group or compound.

Additional aspects will be set forth in part in the description which follows and, in part, will be apparent from the description.

The starting materials useful for making the pharmaceutical compositions of this disclosure are readily commercially available or can be prepared by those skilled in the art.

This disclosure is directed to compositions that include therapy enhancer agents containing moieties of interest conjugated to target agent moieties at specific locations.

In an embodiment, provided is a compound having the structure of formula R-I:

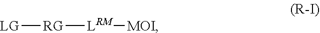

or a salt thereof, wherein:
LG is a group comprising a target binding moiety that binds to a target agent,
RG is a reactive group of formula -L^{LG2}-L^{LG3}-L^{LG4}-L^{RG1}-L^{RG2}-,
   L^{LG2} is —NH—C(O)O—C(R′)₂—, wherein each R′ is independently H or C1-C10 alkyl, wherein
R′ are optionally connected to form a ring;
   L^{LG3} is an optionally substituted aryl ring;
   L^{LG4-} is —NH— or —O—;
   L^{RG1} is —C(O)—, —S(O)—, —OS(O)₂—, or —OP(O)(OR)₂—; and
   L^{RG2}—is a covalent bond or [—C(R″)₂C(R″)═C(R″)]C(O)—, wherein each R″ is independently
   H or C1-C10 alkyl, wherein any two R″ are optionally connected to form a ring;
L^{RM} is a linker; and
MOI is a moiety of interest.

### Targets

Those skilled in the art after reading the present disclosure will appreciate that provided technologies herein are useful for conjugating various target agents to many types of moieties of interest. In some embodiments, provided technologies are particularly useful for conjugating protein agents with various moieties of interest. In some embodiments, target agents are or include a protein agent, a nucleic acid, or a combination thereof.

In some embodiments, a target agent is or includes a protein agent. In some embodiments, a target agent is a protein agent. In some embodiments, a target agent is a natural protein in a cell, tissue, organ or organism. In some embodiments, a target agent is an endogenous protein. In some embodiments, a target agent is an exogenous protein. In some embodiments, a target agent is a manufactured protein, e.g., a protein produced using various biotechnologies. In some embodiments, a target agent is an antibody agent. In some embodiments, a target agent is an antibody useful as therapeutics. Various such antibodies are known in the art and can be utilized as target agents. In some embodiments, an antibody is a monoclonal antibody. In some embodiments, an antibody is a polyclonal antibody. In some embodiments, an antibody is an IgG antibody. In some embodiments, an antibody is IVIG (in some embodiments, pooled from healthy donors). In some embodiments, a protein includes a Fc region. In some embodiments, an antibody includes a Fc region. In some embodiments, a Fc region includes a single heavy chain or a fragment thereof. In some embodiments, a Fc region includes two heavy chains or fragments thereof. In some embodiments, an antibody is a human antibody. In some embodiments, an antibody is a chimeric antibody. In some embodiments, an antibody is a humanized antibody. In some embodiments, an antibody is a mouse antibody.

In some embodiments, when characterizing polyclonal antibody agents or IVIG agents, either before, during or after conjugation, digestions are performed, e.g., enzyme digestions using IdeZ, IdeS, etc., so that certain regions of antibodies (e.g., Fab) are removed to provide compositions with improved homogeneity for characterization (e.g., by MS).

In some embodiments, an antibody is a therapeutic antibody, e.g., an FDA-approved antibody for therapeutic uses. In some embodiments, a therapeutic antibody is useful for treating cancer. In some embodiments, an antibody is adalimumab, alemtuzumab, atezolizumab, avelumab, ipilimumab, cetuximab, daratumumab, dinutuximab, elotuzumab, ibritumomab tiuxetan, imgatuzumab, infliximab, ipilimumab, necitumumab, obinutuzumab, ofatumumab, pertuzumab, reslizumab, rituximab, trastuzumab, mogamulizumab, AMP-224, FS-102, GSK-2857916, ARGX-111, ARGX-110, AFM-13, APN-301, BI-836826, BI-836858, enoblituzumab, otlertuzumab, veltuzumab, KHK-4083, BIW-8962, ALT-803, carotuximab, epratuzumab, inebilizumab, isatuximab, margetuximab, MOR-208, ocaratuzumab, talacotuzumab, tremelimumab, benralizumab, lumiliximab, MOR-208, Ifibatuzumab, GSK2831781, SEA-CD40, KHK-2823, or BI836858. In some embodiments, an antibody is rituximab, basiliximab, infliximab, cetuximab, siltuximab, dinutuximab, altertoxaximab, daclizumab, palivizumab, trastuzumab, alemtuzumab, omalizumab, efalizumab, bevacizumab, natalizumab, tocilizumab, eculizumab, mogamulizumab, pertuzumab, obinutuzumab, vedolizumab, pembrolizumab, mepolizumab, elotuzumab, daratumumab, ixekizumab, reslizumab, and atezolizumab, adalimumab, panitumumab, golimumab, ustekinumab, canakinumab, ofatumumab, denosumab, ipilimumab, belimumab, raxibacumab, ramucirumab, nivolumab, secukinumab, evolocumab, alirocumab, necitumumab, brodalumab, or olaratumab. In some embodiments, an antibody is daratumumab. In some embodiments, an antibody is cetuximab. In some embodiments, a provided compound or agent including an antibody agent moiety is useful for treating a condition, disorder or disease that may be treated by the antibody agent.

Antibodies may be prepared in a number of technologies in accordance with the present disclosure. In some embodiments, antibodies may have engineered structures compared to natural immunoglobulins. In some embodiments, antibodies may include certain tags for purification, identification, assessment, etc. In some embodiments, antibodies may contain fragments (e.g., CDR and/or Fc, etc.) and not full immunoglobulins. Those skilled in the art appreciate that when a site of an antibody is recited in the present disclosure (e.g., K246, K248, K288, K290, K317, etc.; unless indicated otherwise, human antibody per EU numbering), an amino acid residue may not be at the exact numbered site but may be at a site that corresponds to that numbered site per, e.g., EU numbering and/or sequence homology (e.g., homologues of the same or different species).

As those skilled in the art will appreciate, provided technologies among other things can provide directed conjugation with native targets, e.g., native antibodies. In some embodiments, target agents are or include native antibody agents. In some embodiments, target agents are or include engineered antibody agents. In some embodiments, target agents, e.g., antibodies, include no engineered unnatural amino acid residues.

### Target Binding Moieties

In some embodiments of formulae (LG-I) and (R-I):
LG is R^{LG}-L^{LG};
R^{LG} is

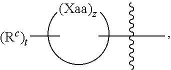

R^{c}—(Xaa)z-, a nucleic acid moiety, or a small molecule moiety;
each Xaa is independently a residue of an amino acid or an amino acid analog;

t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-;
each of L^{LG1}, L^{LG2}, L^{LG3}, and L^{LG4} is independently a covalent bond, or a bivalent optionally substituted, linear or branched C₁₋₁₀₀ group including one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiment, LG is or includes a target binding moiety that binds to a target agent, wherein the target agent is an antibody agent.

In some embodiments, LG is or includes a target binding moiety that binds to a Fc region, and/or R^{LG} is or includes DCAWXLGELVWCT (SEQ ID NO:2), wherein the two cysteine residues optionally form a disulfide bond, and X is an amino acid residue.

In some embodiments, LG is or includes a target binding moiety having the structure of A-1 to A-50:

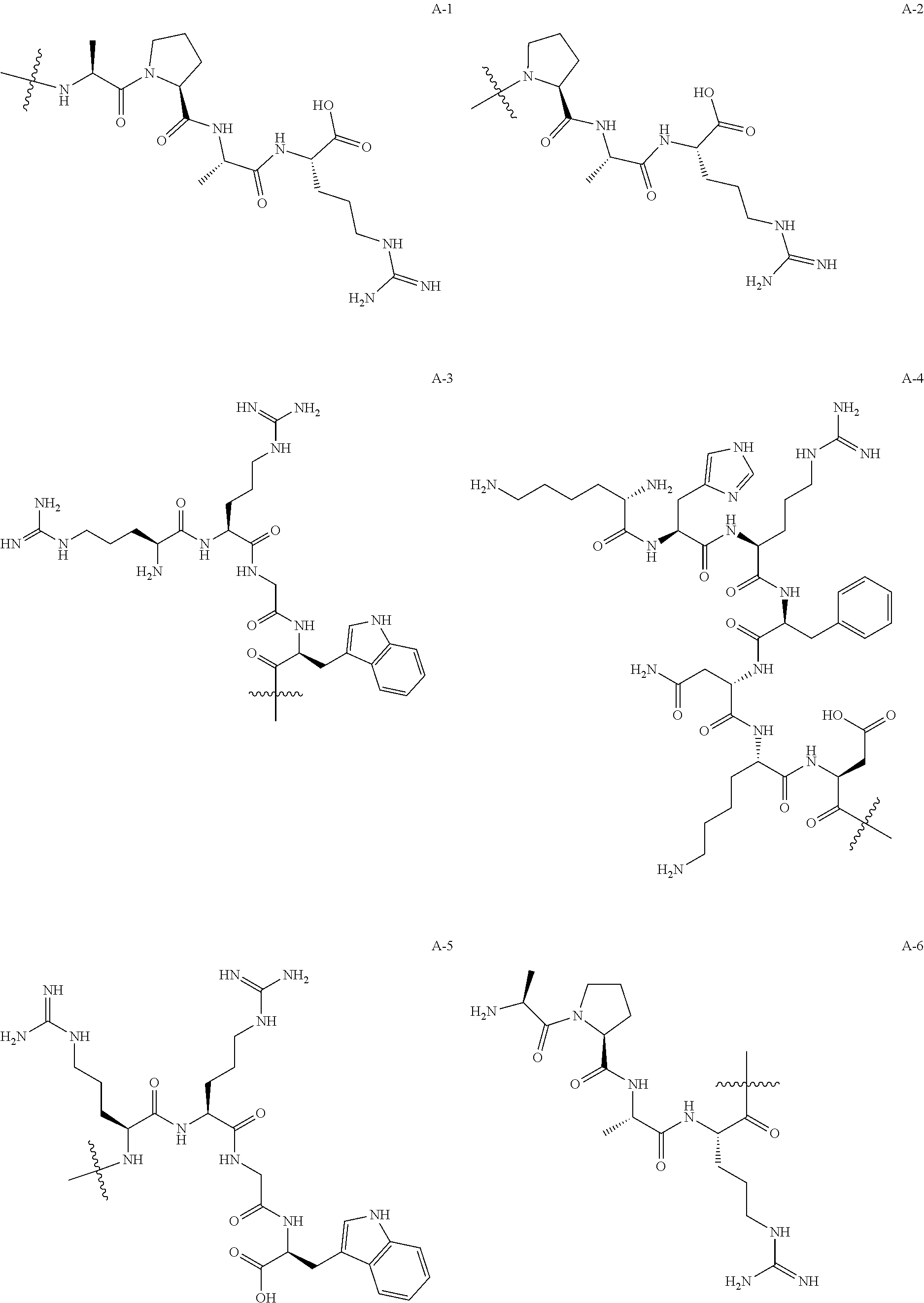

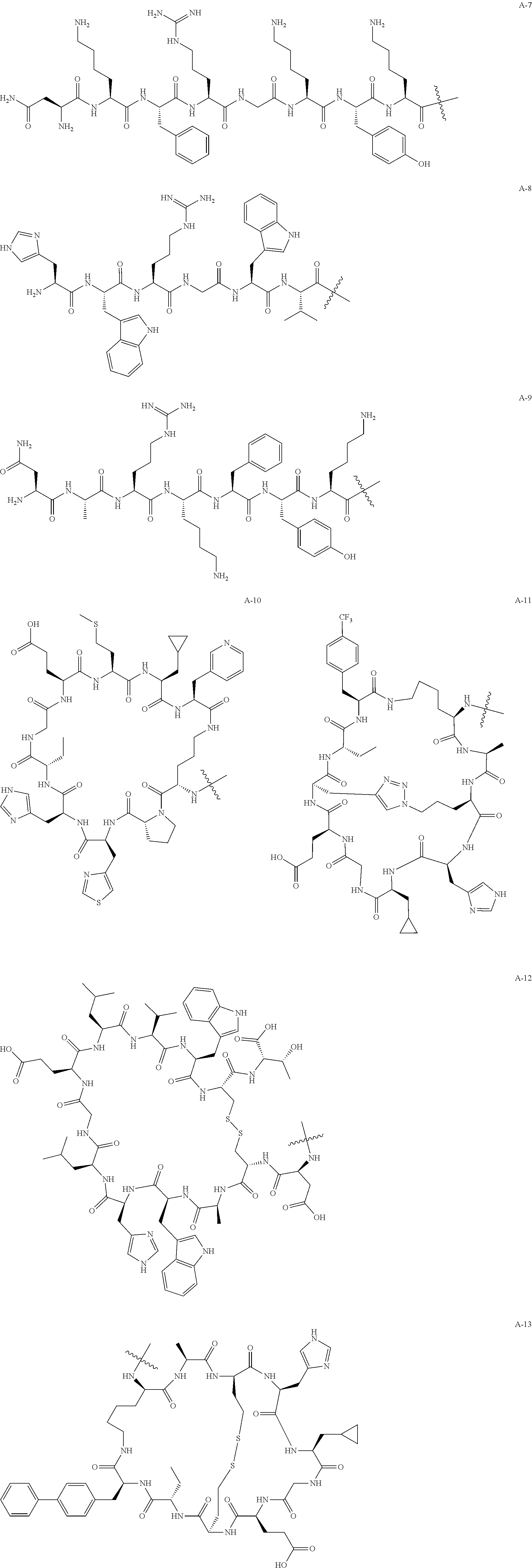

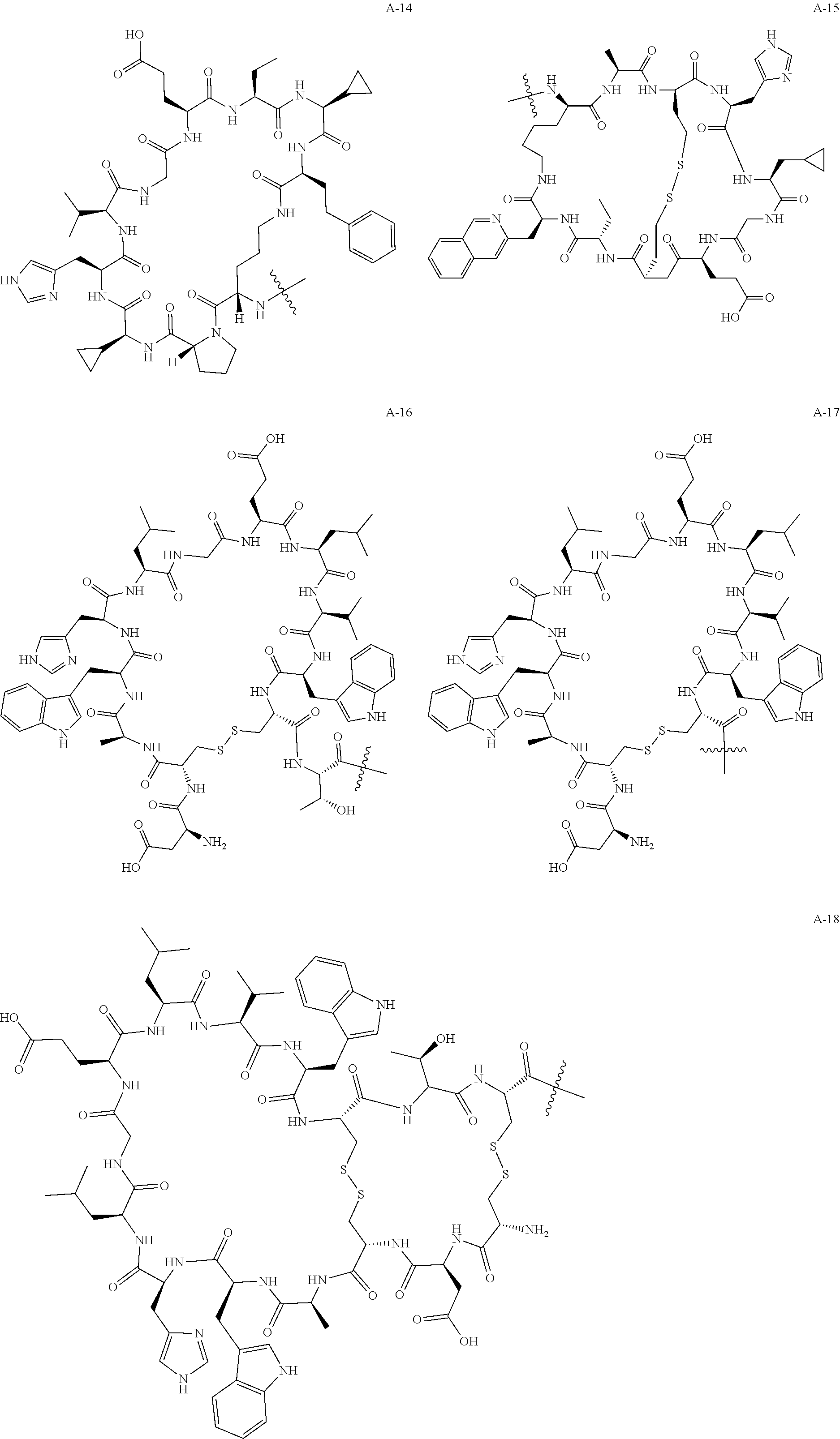

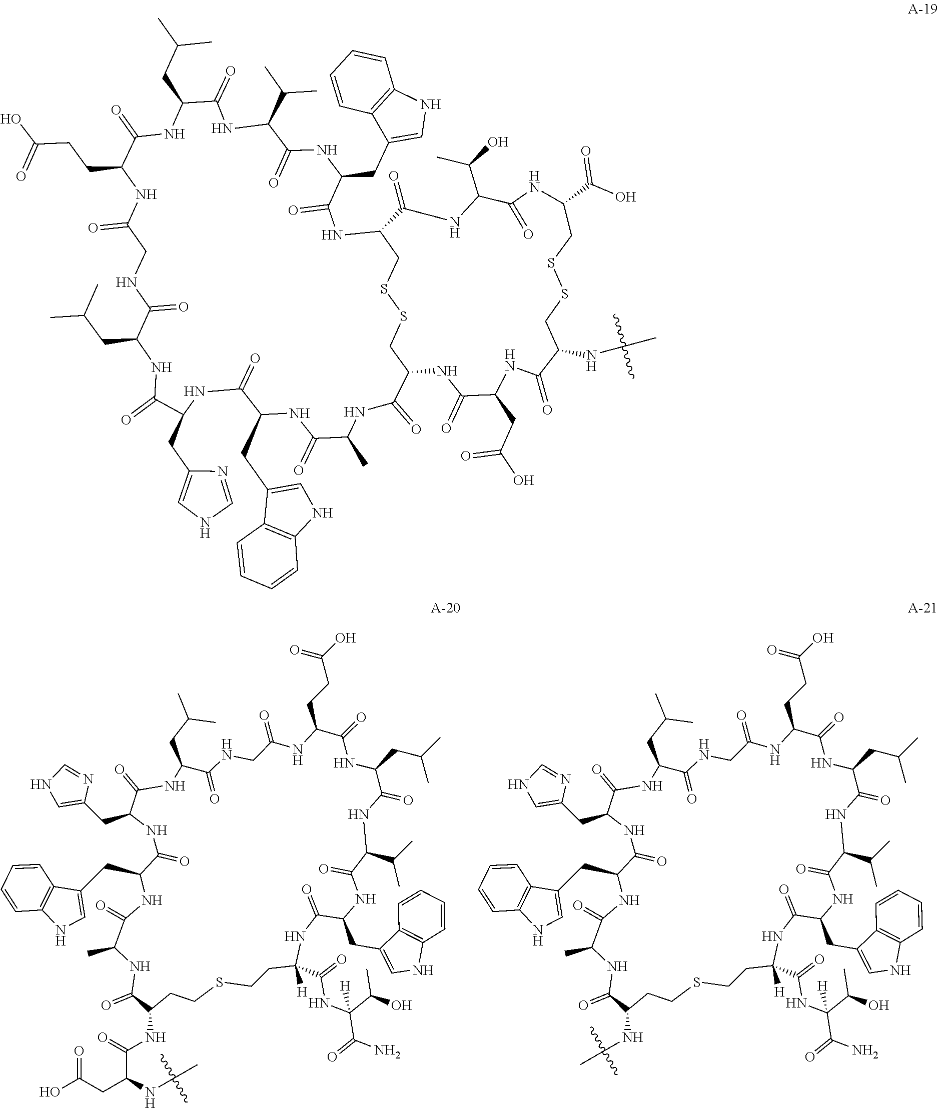

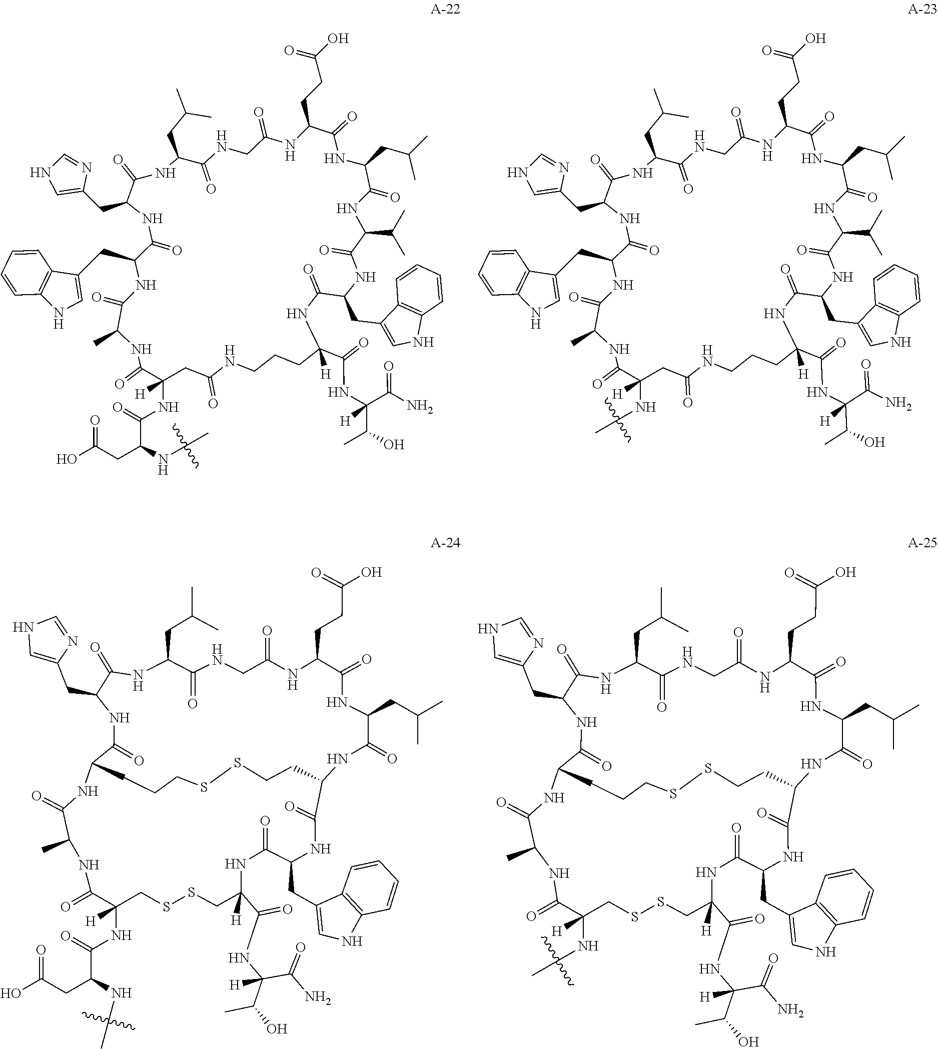

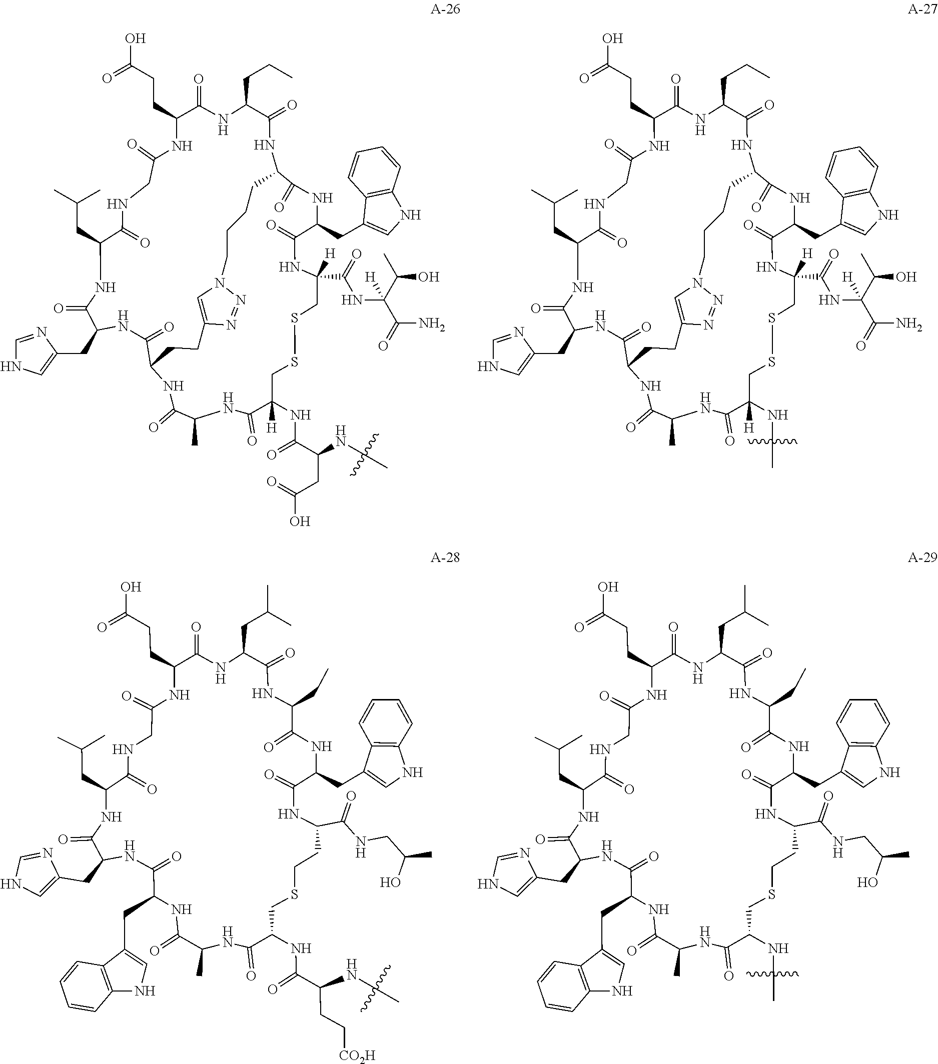

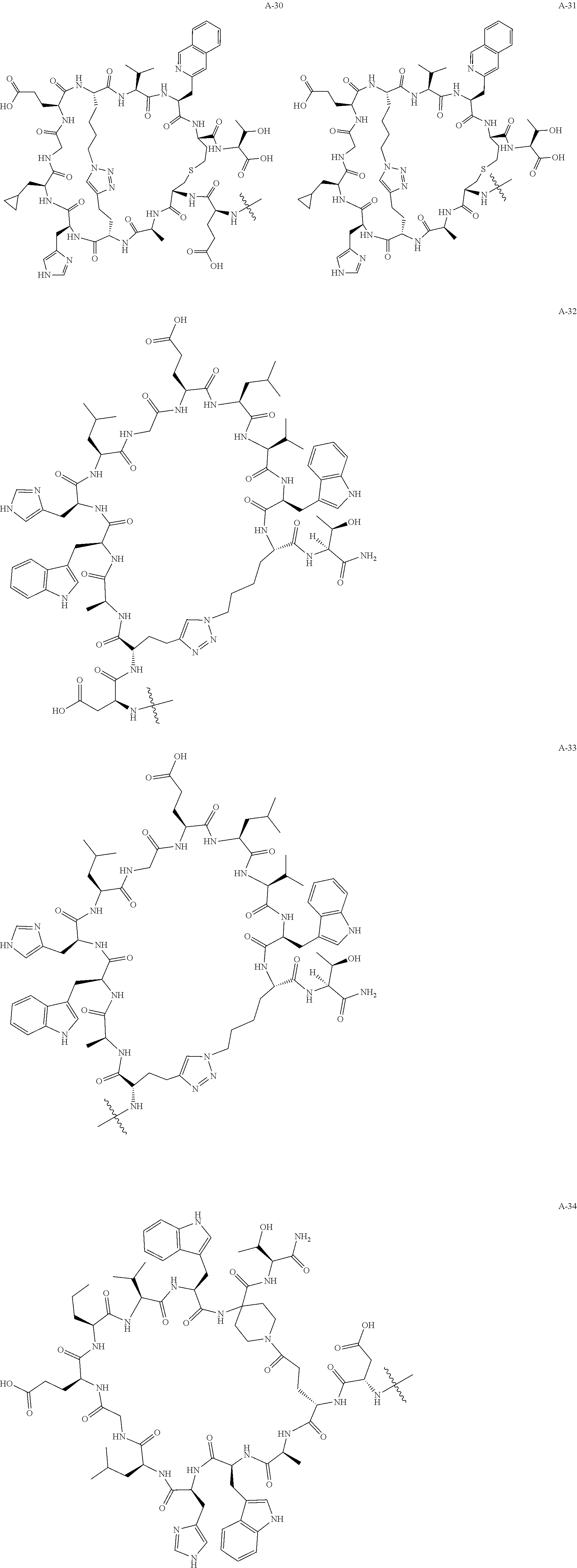

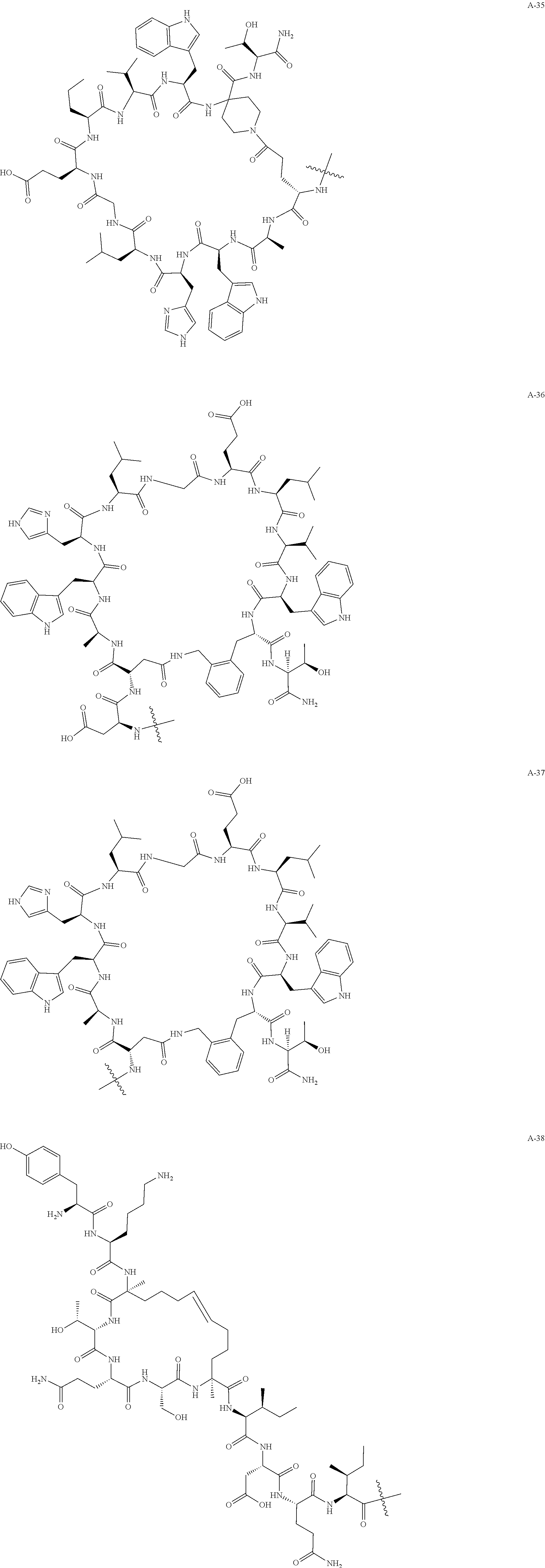

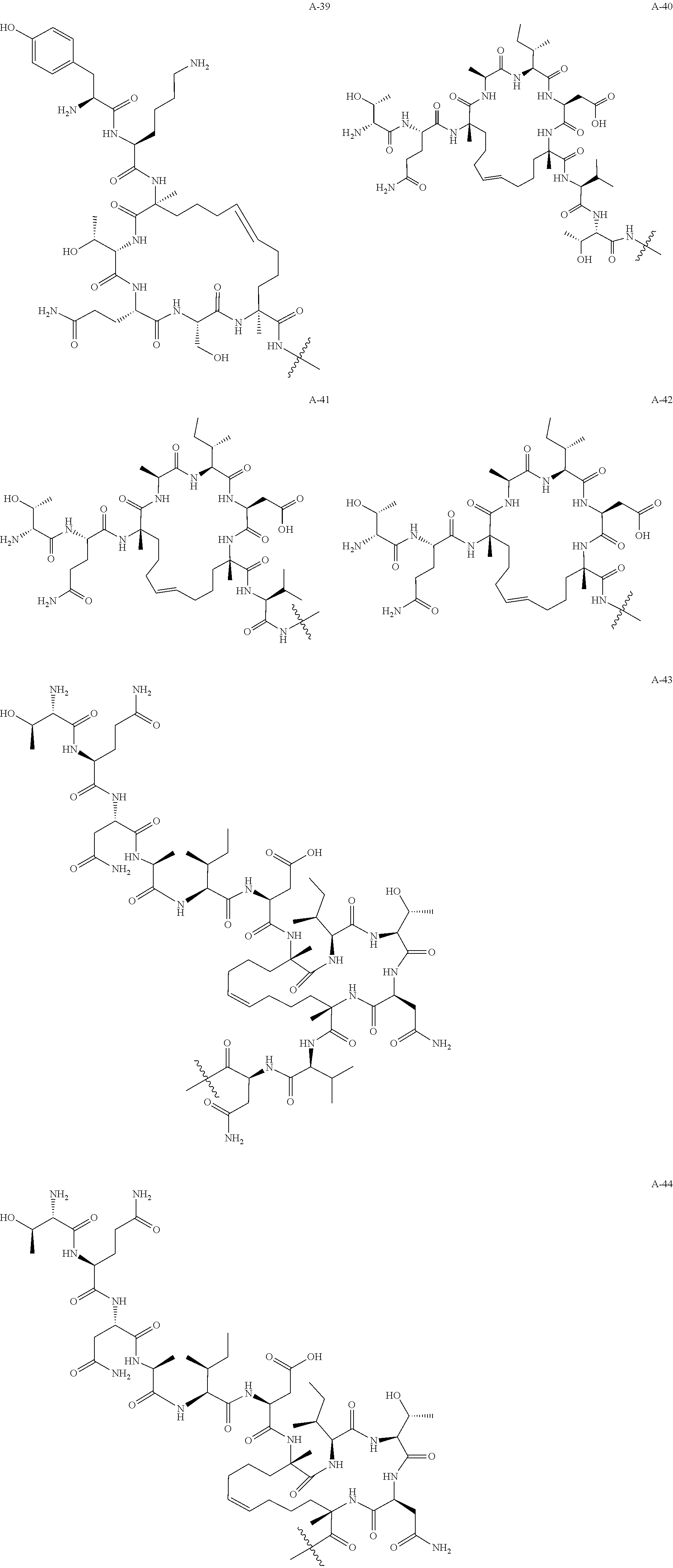

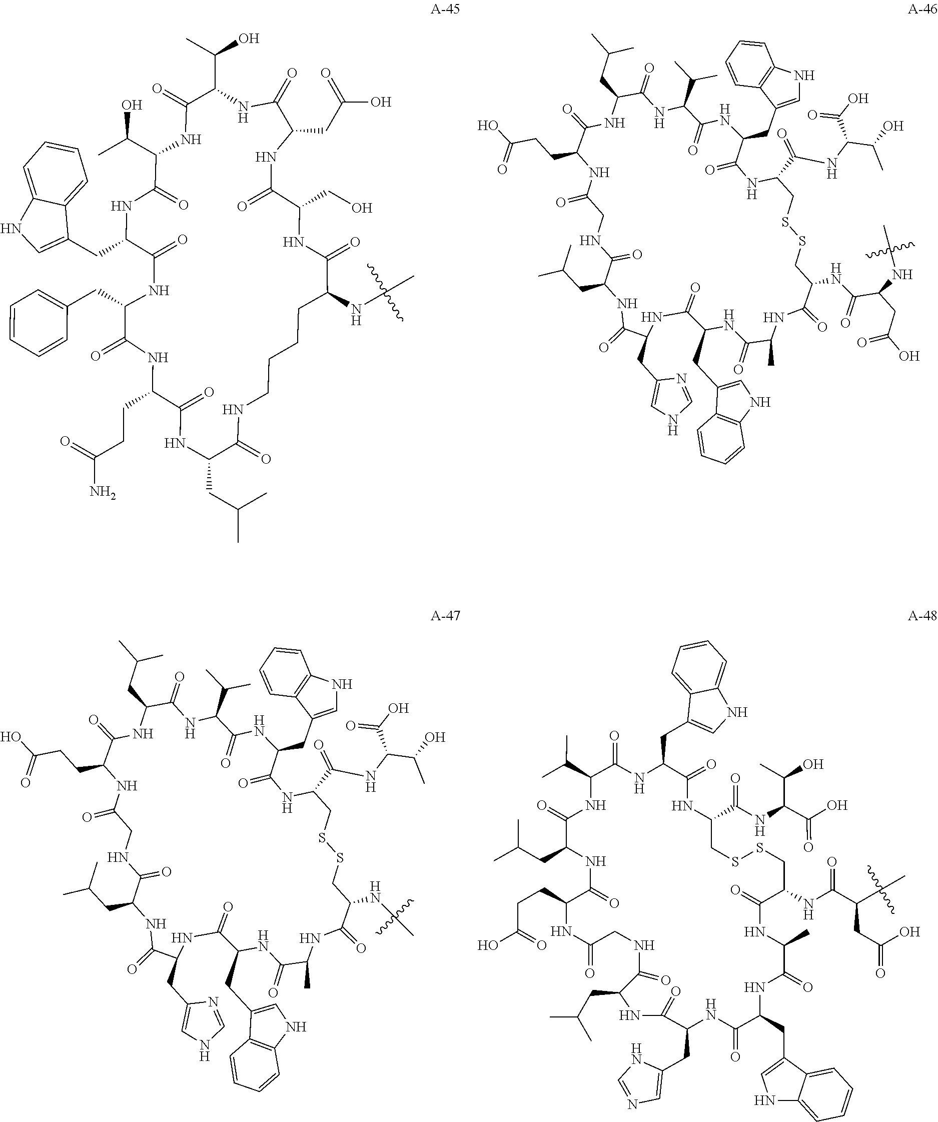

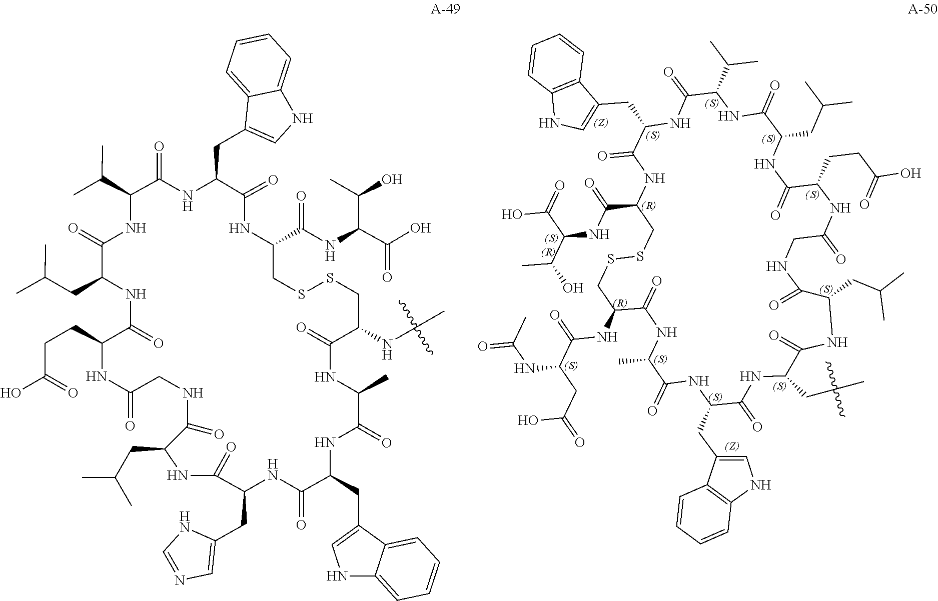

### MOIETIES OF INTEREST

Those skilled in the art reading the present disclosure will appreciate that various types of moieties of interest can be utilized for various purposes in accordance with the present disclosure.

In some embodiments, moieties of interest are or include detectable moieties. Among other things, such moieties can be useful for detection, quantification, diagnosis, treatment, etc. In some embodiments, a moiety of interest is or includes a radioactive label. In some embodiments, a moiety of interest is or includes a label that can be detected through spectroscopy. In some embodiments, a moiety of interest is or includes a fluorophore such as FITC moiety.

A moiety of interest may be a moiety having affinity to a particular target implicated in a medical condition, disorder, or disease. In some embodiments, moieties of interest are or include therapeutic agent moieties. In some embodiments, a moiety of interest is or includes a drug moiety, e.g., a drug moiety in an antibody-drug conjugate. In some embodiments, a moiety of interest is or includes a toxic agent. In some embodiments, a moiety of interest is or includes a cytotoxic agent. In some embodiments, a moiety of interest is or includes an anti-cancer agent. In some embodiments, an anti-cancer agent is a chemotherapeutic agent.

In some embodiments, moieties of interest are or include moieties that can interact and/or recruit other agents, such as proteins, nucleic acids, cells, etc. In some embodiments, moieties of interest interact with proteins expressed by certain cell types, e.g., immune cells, disease cells, etc. In some embodiments, moieties of interest are immune cell binders. In some embodiments, moieties of interest recruit immune cells. In some embodiments, moieties of interest trigger, promote and/or enhance one or more immune activities, e.g., for removing, killing, and/or inhibiting desired targets (e.g., cancer cells, antigens, etc.). In some embodiments, moieties of interest interact, recruit and/or bind to disease cells, and trigger, promote and/or enhance removing, killing, and/or inhibiting disease cells.

In some embodiments, a moiety of interest is or includes a small molecule agent (e.g., one can bind specifically to its protein targets, cells targets, etc.). In some embodiments, a moiety of interest is or includes a peptide or protein agent (e.g., scFv, a peptide binder to specific target, etc.). In some embodiments, a moiety of interest is or includes a nucleic acid agent (e.g., an oligonucleotide, mRNA, etc.). In some embodiments, a moiety of interest is or includes a carbohydrate agent. In some embodiments, a moiety of interest is or includes a lipid agent.

In some embodiments, a moiety of interest is or includes a protein complex (e.g., Fab). In some embodiments, a moiety of interest is or includes a fluorophore. In some embodiments, a moiety of interest is or includes a cytotoxic small molecule agent. In some embodiments, a moiety of interest is or includes a cytotoxic peptide agent.

In some embodiments, a moiety of interest is an adjuvant. Those skilled in the art will appreciate various adjuvants can be utilized as moieties of interest in accordance with the present disclosure. In some embodiments, an adjuvant is one described in US 2019/0015516, which is incorporated herein in its entirety by reference. In some embodiments, a moiety of interest stimulates an immune system.

In some embodiments, a moiety of interest is or includes a particle. In some embodiments, a particle is or includes a nanoparticle.

In some embodiments, a moiety of interest is or includes a nucleic acid moiety. In some embodiments, a moiety of interest is or includes an oligonucleotide. In some embodiments, a moiety of interest is or includes an aptamer.

In some embodiments, a moiety of interest is an antibody agent. In some embodiments, a moiety of interest is or includes an antibody fragment. In some embodiments, a moiety of interest is an antibody agent moiety that does not contain a region to which a target binding moiety binds. In some embodiments, a moiety of interest is an antibody agent that contains no Fc region. In some embodiments, a moiety of interest is or includes a scFv. In some embodiments, a scFv is for a different antigen than an antibody target agent.

In some embodiments, moieties of interest are or include reactive moieties, particularly those reaction partners for bio-orthogonal reactions. Suitable reactive moieties, including those for bio-orthogonal reactions, are widely known in the art and can be utilized herein. In some embodiments, a bio-orthogonal reaction is a cycloaddition reaction, e.g., click chemistry. In some embodiments, a moiety of interest is or includes —N₃. In some embodiments, a moiety of interest is or includes an alkyne.

In some embodiments, a moiety of interest may be a moiety that binds to a SARS-COV-2 virus that is implicated in the COVID-19 disease. For example, the moiety that binds to a SARS-COV-2 virus may be a polypeptide disclosed in L. Cao et al., “De novo design of picomolar SARS-COV-2 mini-protein inhibitors” Science 370, 426-431 (2020), which is incorporated herein in its entirety by reference. Such a polypeptide moiety may result in binding to SARS-COV-2 spike proteins, inhibition, reduction and prevention of binding and/or infection of cells, inhibition, killing, and removal of SARS-COV-2 viruses and/or cells infected thereby, etc. Various moieties of interest that interact with the SARS-COV-2 virus are described in International Patent Application No. PCT/US21/24186 filed Mar. 25, 2021, U.S. Provisional Patent Application No. 63/146584 filed Feb. 6, 2021, and U.S. Provisional Patent Application No. 63/182098 filed Apr. 30, 2021, each of which applications is incorporated herein in its entirety by reference.

In some embodiments, a moiety of interest improves one or more properties and/or activities of a target agent. In some embodiments, a moiety of interest is or includes a stability enhancer. In some embodiments, a moiety of interest improves one or more pharmacodynamic and/or pharmacokinetic properties of a target agent.

In some embodiments, at least one of the following conditions is met:
(a) the moiety of interest is or includes a therapeutic agent;
(b) the moiety of interest is or includes a moiety that can bind to a protein, nucleic acid or a cell; and/or
(c) the moiety of interest is or includes a reactive moiety suitable for a bio-orthogonal reaction.

In some embodiments, MOI is or includes a therapeutic agent moiety; and/or MOI is or includes an antibody agent.

### Linking Groups

In some embodiments, moieties are optionally connected to each other through linker moieties. For example, in some embodiments, a reactive group, e.g., RG, is connected to a moiety of interest, e.g., MOI, through a linker, e.g., L^{RM}. In some embodiments, a moiety, e.g., LG, may also include one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, L^{LM} includes one or more —[(CH₂)ₙ—O]ₘ—, wherein each n is independently 1-20, and m is 1-100.

In some embodiments, L^{RM} the linker includes one or more —[(CH₂)ₙ—O]ₘ—, wherein each n is independently 1-20, and m is 1-100.

### Reactive Groups

In some embodiments, provided compounds, e.g., those useful as reaction partners, include reactive groups (e.g., RG). As exemplified herein, in many embodiments, in provided compounds reactive groups (e.g., RG) are located between first groups (e.g., LG) and moieties of interest (e.g., MOI), and are optionally and independently linked to first groups and moieties of interest via linkers. In some embodiments, RG is a reaction group as described herein.

In some embodiments, as demonstrated herein, reactive groups when utilized in compounds that include no target binding moieties react slowly and provide low level of, in some embodiments, substantially no conjugation of moieties of interest with target agents. As demonstrated herein, combination of reactive groups with target binding moieties in the same compounds, e.g., as in compounds of formula R-I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in provided compounds can react with various types of groups in target agents.

In some embodiments, reactive groups in provided compounds selectively react with amino groups of target agents, e.g., —NH₂ groups on side chains of lysine residues of proteins. In some embodiments, reactive groups when utilized in provided compounds, e.g., those of formula R-I or salts thereof, selectively react with particular sites of target agents, e.g., as shown in examples herein, one or more of K246, K248, K288, K290, K317, etc. of IgG1, K251, K 253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without target binding moieties, conjugation occurs at light chain sites more than heavy chain sites.

In some embodiments, a reactive group, e.g., RG, is or includes an ester group. In some embodiments, a reactive group, e.g., RG, is or includes an electrophilic group, e.g., a Michael acceptor.

In some embodiments, RG is a reactive group of formula -L^{LG2}-L^{LG3}-L^{LG4}-L^{RG1}-L^{RG2}-, wherein
L^{LG2} is —NH—C(O)O—C(R′)₂—, wherein each R′ is independently H or C1-C10 alkyl, wherein R′ are optionally connected to form a ring;
L^{LG3} is an optionally substituted aryl ring;
L^{LG4}- is —NH— or —O—;
L^{RG1} is —C(O)—, —S(O)—, —OS(O)₂—, or —OP(O)(OR)₂—; and
L^{RG2}— is a covalent bond or [—C(R″)₂C(R″)═C(R″)]C(O)—, wherein each R″ is independently H or C1-C10 alkyl, wherein any two R″ are optionally connected to form a ring.

In some embodiments, RG is or comprises a reactive group having the following formula:

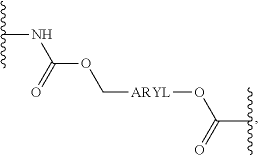

wherein ARYL is a substituted or unsubstituted para-phenylene ring. In some embodiments, ARYL may have the structure of

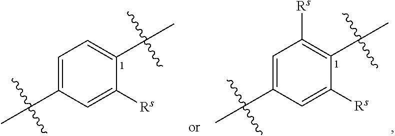

wherein R^{s} is independently chosen at each occurrence from halogen, —NO₂, —F, -L-R′, —C(O)-L-R′, —S(O)-L-R′, —S(O)₂-L-R′, and —P(O)(-L-R′)₂, and R′ is H or C₁-C₆calkyl.

In some embodiments, the reactive group is or comprises has one of the following formulae:

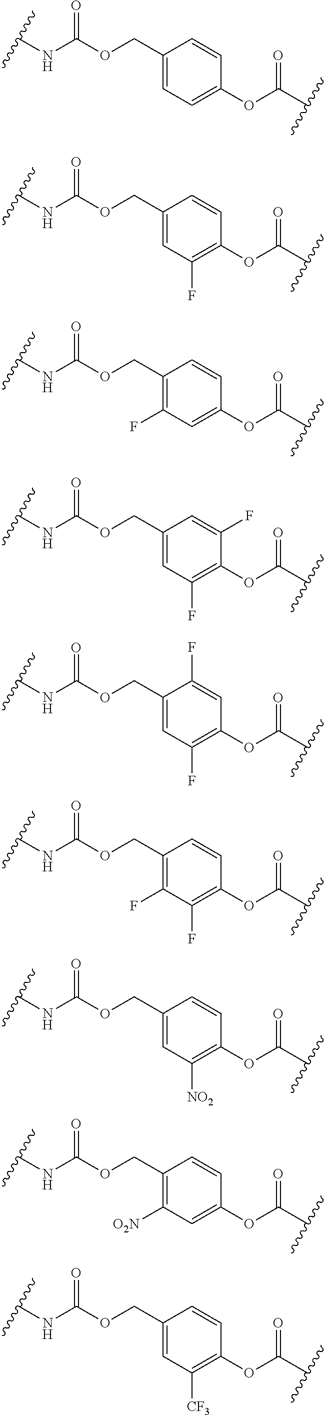

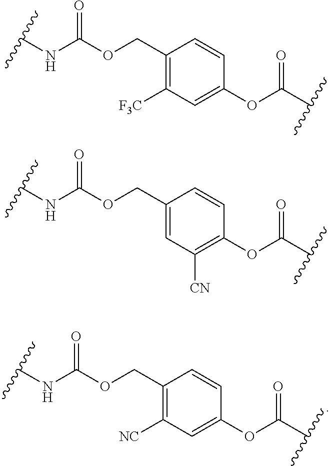 [SMILES: *NC(=O)OCc1ccc(OC(*)=O)cc1C(F)(F)F ; *NC(=O)OCc1ccc(OC(*)=O)c(C#N)c1 ; *NC(=O)OCc1ccc(OC(*)=O)cc1C#N]

In some embodiments, RG is or comprises a reactive group having the following formula:

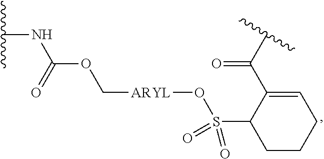

wherein ARYL is a substituted or unsubstituted para-phenylene ring. In some embodiments, ARYL may have the structure of

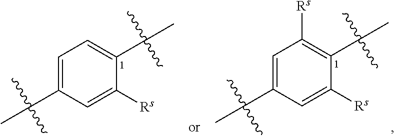

wherein R^{s} is independently chosen at each occurrence from halogen, —NO₂, —F, -L-R′, —C(O)-L-R′, —S(O)-L-R′, —S(O)₂-L-R′, and —P(O)(-L-R′)₂, and R′ is H or C₁-C₆alkyl.

In some embodiments, the reactive group is or comprises has one of the following formulae:

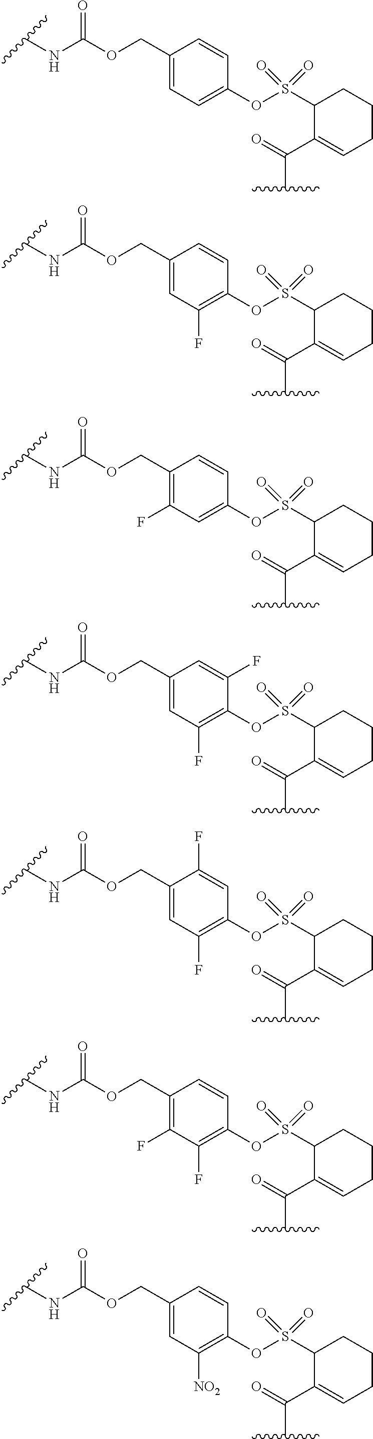

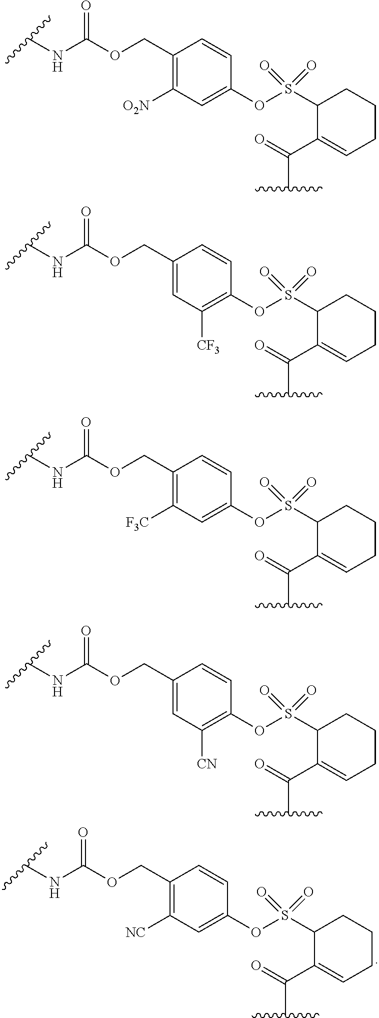 [SMILES: *NC(=O)OCc1ccc(OS(=O)(=O)C2CCCC=C2C(*)=O)cc1[N+](=O)[O-] ; *NC(=O)OCc1ccc(OS(=O)(=O)C2CCCC=C2C(*)=O)c(C(F)(F)F)c1 ; *NC(=O)OCc1ccc(OS(=O)(=O)C2CCCC=C2C(*)=O)cc1C(F)(F)F ; *NC(=O)OCc1ccc(OS(=O)(=O)C2CCCC=C2C(*)=O)c(C#N)c1 ; *NC(=O)OCc1ccc(OS(=O)(=O)C2CCCC=C2C(*)=O)cc1C#N]

### Compositions

In some embodiments, provided is a composition comprising one or more of the above compounds.

In some embodiments, the composition may include:
a first compound having the structure of formula (P-II):

P—N-L^{PM}-MOI (P-II)
wherein:
P—N is a protein agent moiety comprising a lysine residue;
L^{PM} is a linker; and
MOI is a moiety of interest; and
a second compound having the structure:

LG-OH (LG-I)
wherein LG is a group comprising a target binding moiety that binds to a target agent.

In some embodiments, the composition may further include:
a third compound having the formula (R-I):

LG-RG-L^{RM}-MOI (R-I)
LG is a group comprising a target binding moiety that binds to a target agent, which is identical to LG in formula (LG-I);
RG is a reactive group of formula -L^{LG2}-L^{LG3}-L^{LG4}-L^{RG1}-L^{RG2}-, wherein
   L^{LG2} is —NH—C(O)O—C(R′)₂—, wherein each R′ is independently H or C1-C10 alkyl, wherein R′ are optionally connected to form a ring;
   L^{LG3} is an optionally substituted aryl ring;
   L^{LG4}- is —NH— or —O—;
   L^{RG1} is —C(O)—, —S(O)—, —OS(O)₂—, or —OP(O)(OR)₂—; and
   L^{RG2}- is a covalent bond or [—C(R″)₂C(R″)═C(R″)]C(O)—, wherein each R″ is independently H or C1-C10 alkyl, wherein any two R″ are optionally connected to form a ring;
L^{RM} is a linker, which is identical to in formula (P-II); and
MOI is a moiety of interest;
a fourth compound having the formula (R-III):

HO-RG-L^{RM}-MOI (R-III)
or a combination thereof.

In some embodiment, the compositions may include the first and second compounds in equimolar amount. In some embodiments, the amount of the second compound may be 50 mole percent (mole %) or less based on the total number of moles of the first and second compounds in the composition. In some embodiments, the amount of the second compound may be 50 mole % or less, 45 mole % or less, 40 mole % or less, 35 mole % or less, 30 mole % or less, 25 mole % or less, 20 mole % or less, 15 mole % or less, 10 mole % or less, or 5 mole % or less based on the total number of moles of the first and second compounds in the composition. In some embodiments, the amount of the second compound may be 5% or less, 4% or less, 3% or less, 2% or less, or 1% or less based on the total number of moles of the first and second compounds in the composition. In some embodiments, the amount of the second compound may be 1.0% or less, 0.9% or less, 0.8% or less, 0.7% or less, 0.6% or less, 0.5% or less, 0.4% or less, 0.3% or less, 0.2% or less, 0.1% or less based on the total number of moles of the first and second compounds in the composition. In some embodiments, the amount of the second compound may be 0.10% or less, 0.09% or less, 0.08% or less, 0.07% or less, 0.06% or less, 0.05% or less, 0.04% or less, 0.03% or less, 0.02% or less, 0.01% or less based on the total number of moles of the first and second compounds in the composition. In some embodiments, the amount of the second compound may be 0.010% or less, 0.009% or less, 0.008% or less, 0.007% or less, 0.006% or less, 0.005% or less, 0.004% or less, 0.003% or less, 0.002% or less, 0.001% or less based on the total number of moles of the first and second compounds in the composition. In some embodiments, the amount of the second compound may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the total number of moles of the first and second compounds in the composition. In some embodiments, the amount of the second compound may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the total number of moles of the first and second compounds in the composition. In some embodiments, the amount of the second compound may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the total number of moles of the first and second compounds in the composition.

In some embodiment, the compositions may further include a third compound, a fourth compound, or a combination thereof. In some embodiments, the amount of the third compound, the fourth compound, or the combination thereof may be 5% or less, 4% or less, 3% or less, 2% or less, or 1% or less based on the number of moles of the first compound in the composition. In some embodiments, the amount of the third compound, the fourth compound, or the combination thereof may be 1.0% or less, 0.9% or less, 0.8% or less, 0.7% or less, 0.6% or less, 0.5% or less, 0.4% or less, 0.3% or less, 0.2% or less, 0.1% or less based on the number of moles of the first compound in the composition. In some embodiments, the amount of the third compound, the fourth compound, or the combination thereof may be 0.10% or less, 0.09% or less, 0.08% or less, 0.07% or less, 0.06% or less, 0.05% or less, 0.04% or less, 0.03% or less, 0.02% or less, 0.01% or less based on the number of moles of the first compound in the composition. In some embodiments, the amount of the third compound, the fourth compound, or the combination thereof may be 0.010% or less, 0.009% or less, 0.008% or less, 0.007% or less, 0.006% or less, 0.005% or less, 0.004% or less, 0.003% or less, 0.002% or less, 0.001% or less based on the number of moles of the first compound in the composition. In some embodiments, the amount of the third compound, the fourth compound, or the combination thereof may be 0.0010% or less, 0.0009% or less, 0.0008% or less, 0.0007% or less, 0.0006% or less, 0.0005% or less, 0.0004% or less, 0.0003% or less, 0.0002% or less, 0.0001% or less based on the number of moles of the first compound in the composition. In some embodiments, the amount of the third compound, the fourth compound, or the combination thereof may be 0.00010% or less, 0.00009% or less, 0.00008% or less, 0.00007% or less, 0.00006% or less, 0.00005% or less, 0.00004% or less, 0.00003% or less, 0.00002% or less, 0.00001% or less based on the number of moles of the first compound in the composition. In some embodiments, the amount of the third compound, the fourth compound, or the combination thereof may be 0.000010% or less, 0.000009% or less, 0.000008% or less, 0.000007% or less, 0.000006% or less, 0.000005% or less, 0.000004% or less, 0.000003% or less, 0.000002% or less, 0.000001% or less based on the number of moles of the first compound in the composition. The invention is further illustrated by the following non-limiting examples.

### EXAMPLES

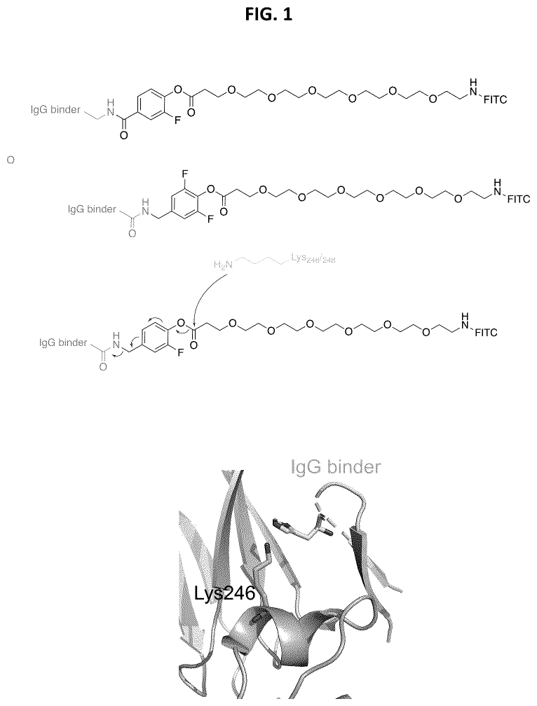

In some embodiments, the target binding moiety may be an immunoglobulin binding moiety including K246 and/or K248, and the process of directed conjugation may be represented by the following diagrams shown in FIG. 1.

In some embodiments, the target binding group may be a linear peptide IgG binder including a directing group having K_{d} of 600 nM:

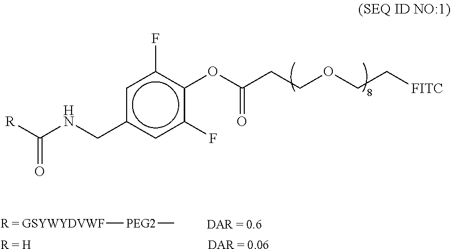

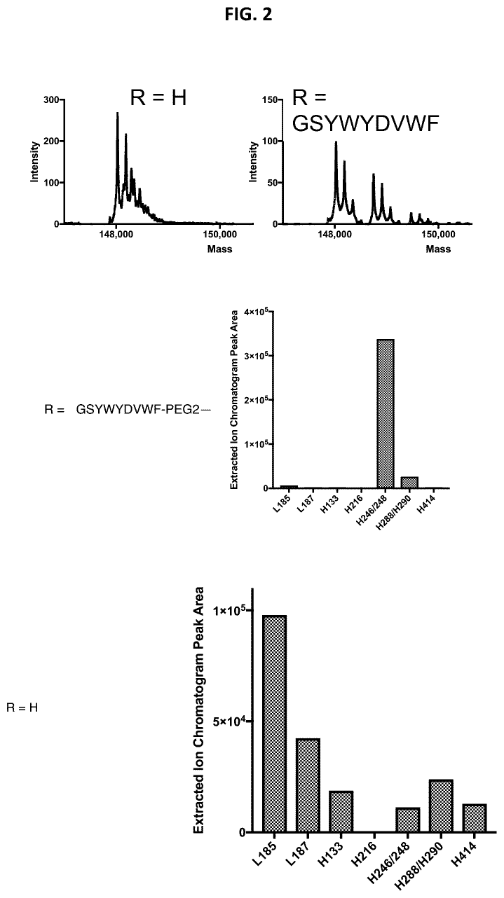

The binding specificity data for the linear peptide IgG binder are shown in FIG. 2.

In some embodiments, the target binding group may be a cyclic peptide IgG binder including a directing group having K_{d} of 15 nM:

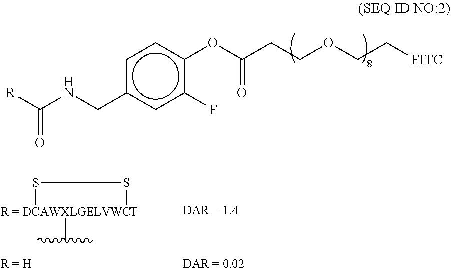

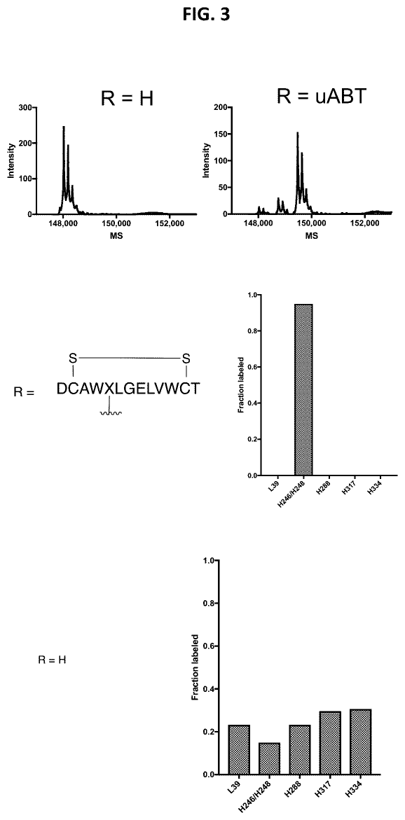

The binding specificity data for the cyclic peptide binder are shown in FIG. 3.

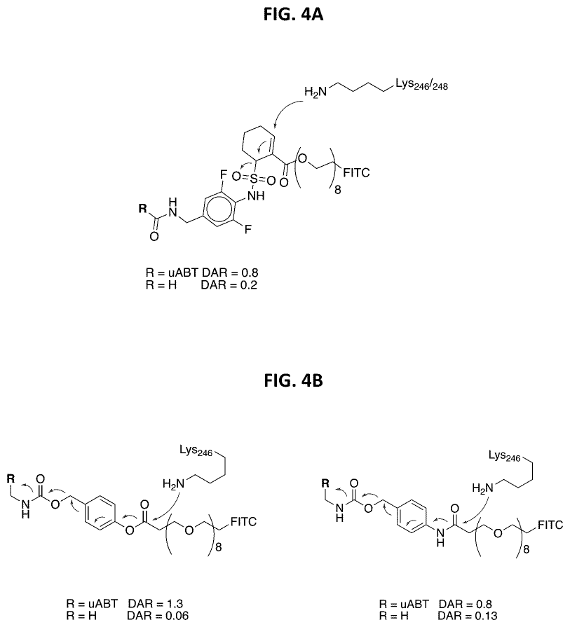

In some embodiments, the compound may have a reactive group that is an aza-Michael acceptor, as shown in FIG. 4A. In some embodiments, the compound may have a reactive group that releases CO2 upon conjugation, as shown in FIG. 4B.

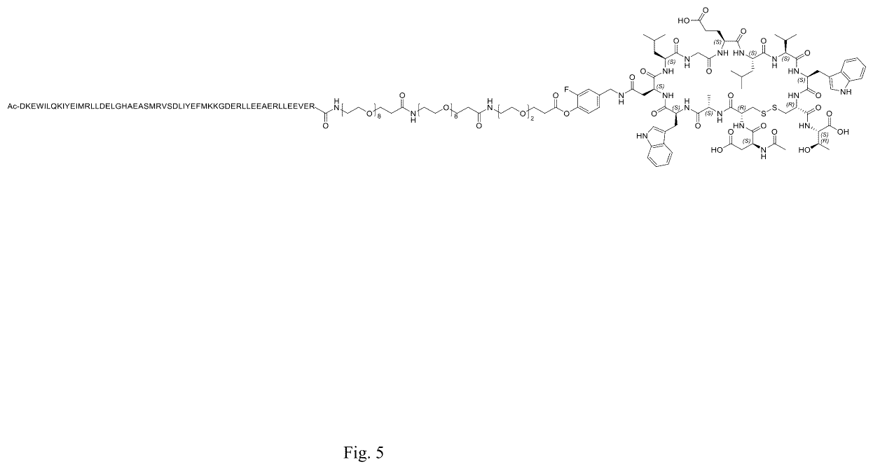

In some embodiments, the compound may have the structure shown in FIG. 5.

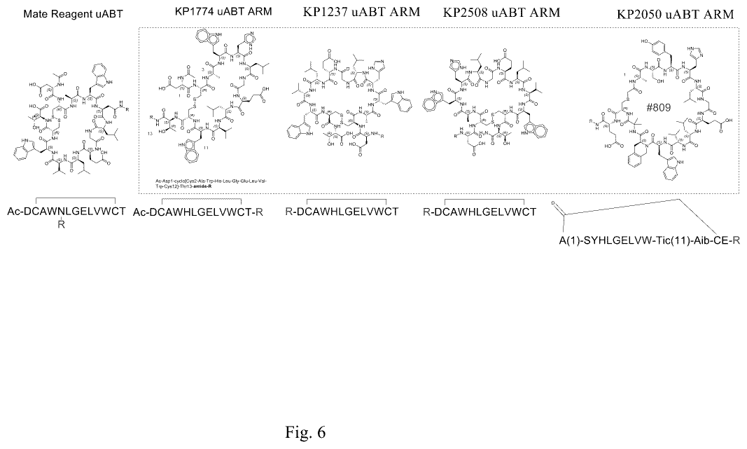

In some embodiments, the target binding group may include one of the following sequences, which are shown in FIG. 6:

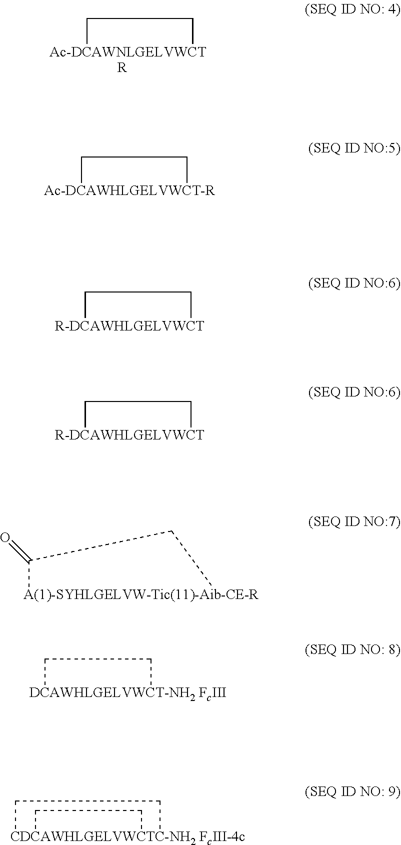

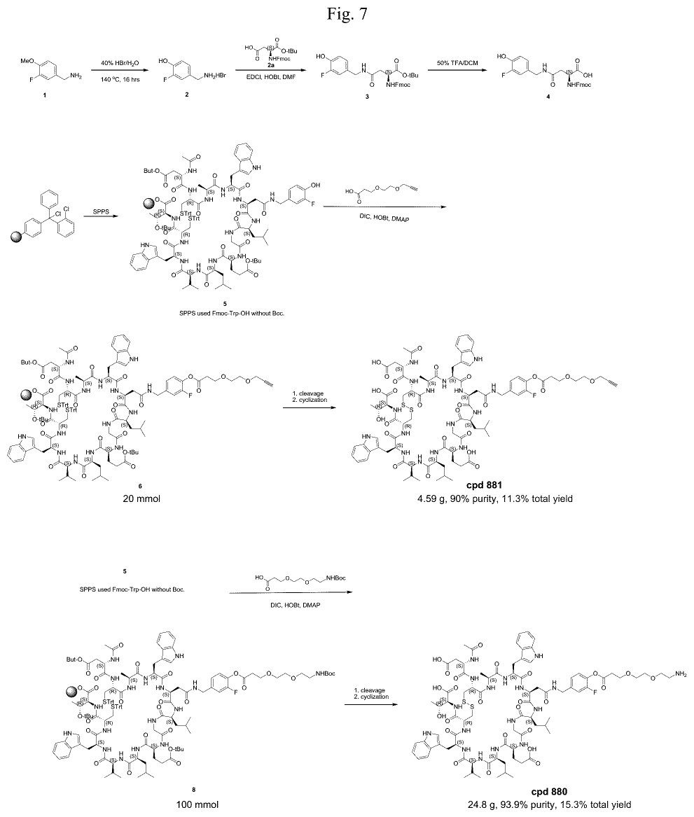

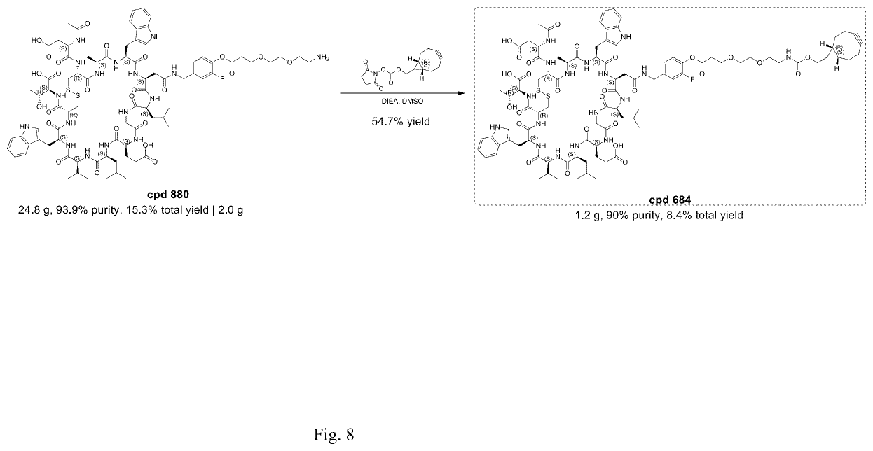

Synthesis of some of these groups are shown in FIGS. 7-8.

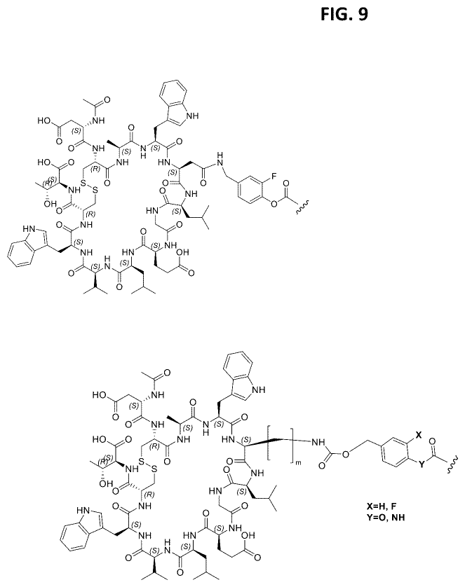

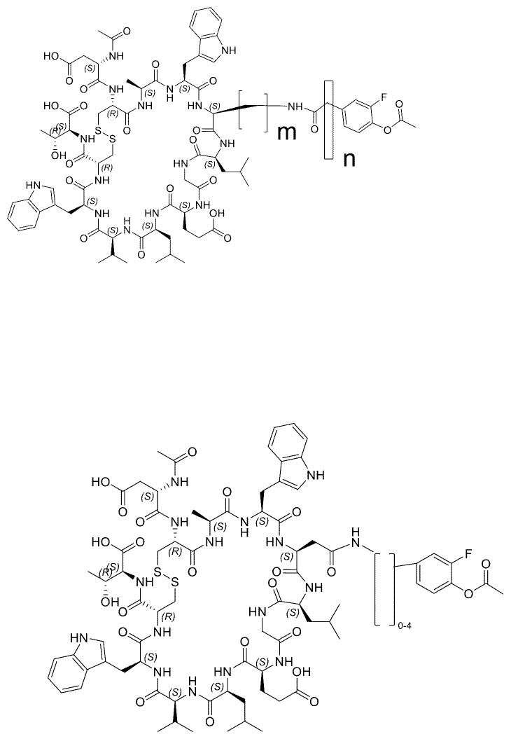

Exemplified LG-RG groups according to some embodiments are shown in FIG. 9.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether there is a specific disclosure herein identifying such a combination.

### ABBREVIATIONS

TABLE 1

| # | Abbreviation | Term |
|---|---|---|
| 1 | FMOC | Fluorenylmethyloxycarbonyl |
| 2 | CTC Resin | 2-Chlorotrityl chloride resin |
| 3 | DCM | Dichloromethane |
| 4 | MeOH | Methanol |
| 5 | DMF | N,N-Dimethylformamide |
| 6 | HBTU | N,N,N′,N′-Tetramethyl-O-(1H-benzotriazol-1- |
| | | yl)uronium hexafluorophosphate |
| 7 | DIEA | N,N-Diisopropylethylamine |
| 8 | TFA | Trifluoroacetic acid |
| 9 | TIS | HBTU (2.85 equiv.) and DIEA (6.00 equiv.) |
| 10 | MeCN | Acetonitrile |
| 11 | AcOH/HOAc | Acetic acid |
| 12 | MS | Mass spectrometry |
| 13 | NMM | 4-Methylmorpholine |
| 15 | HOBt | 1-Hydroxybenzotriazole hydrate |
| 16 | DMAP | 4-(Dimethyl amino)pyridine |
| 17 | LCMS | Liquid chromatography-mass spectrometry |
| 18. | FITC | Fluorescein Isothiocyanate, Isomer I |
| 19 | HPLC | High-performance liquid chromatography |
| 20 | THF | Tetrahydrofuran |
| 21 | NaBH₄ | Sodium borohydride |
| 22 | BOC | tert-butoxycarbonyl protecting group |
| 23 | SPPS | 9-Anthracenecarboxamide, polymer bound |
| 24 | ALLOC | Allyloxycarbonyl protecting group |
| 25 | IgG | Immunoglobulin G |
| 26 | scFv | single-chain variable fragment |
| 27 | SEQ ID | Sequence ID |
| 28 | EDCI | 1-Ethyl-3-(3- |
| | | dimethylaminopropyl)carbodiimide |
| 29 | TIPs | Triisopropylsilane |
| 30 | DIC | N,N′-Diisopropylcarbodiimide |

### Example 1. Procedure for Preparation of Compound 1290

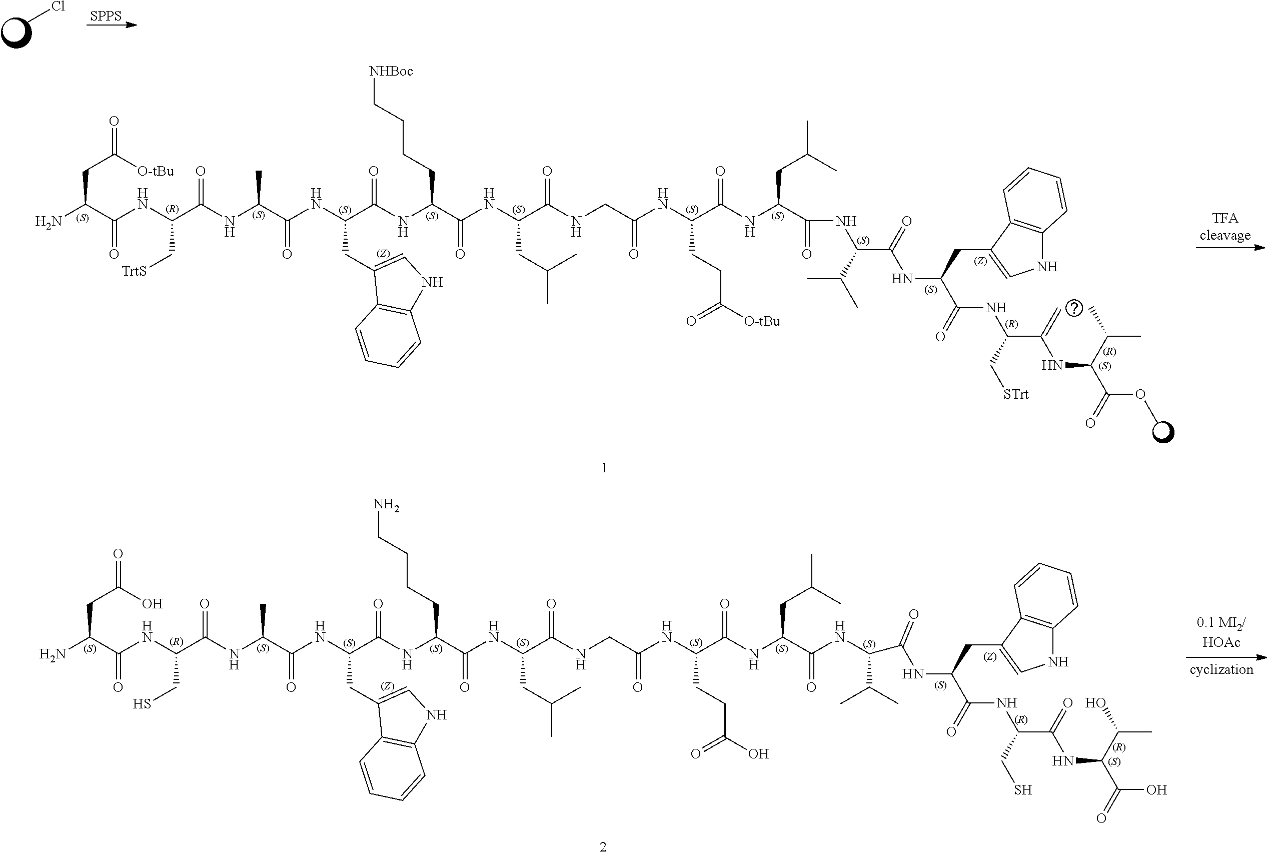

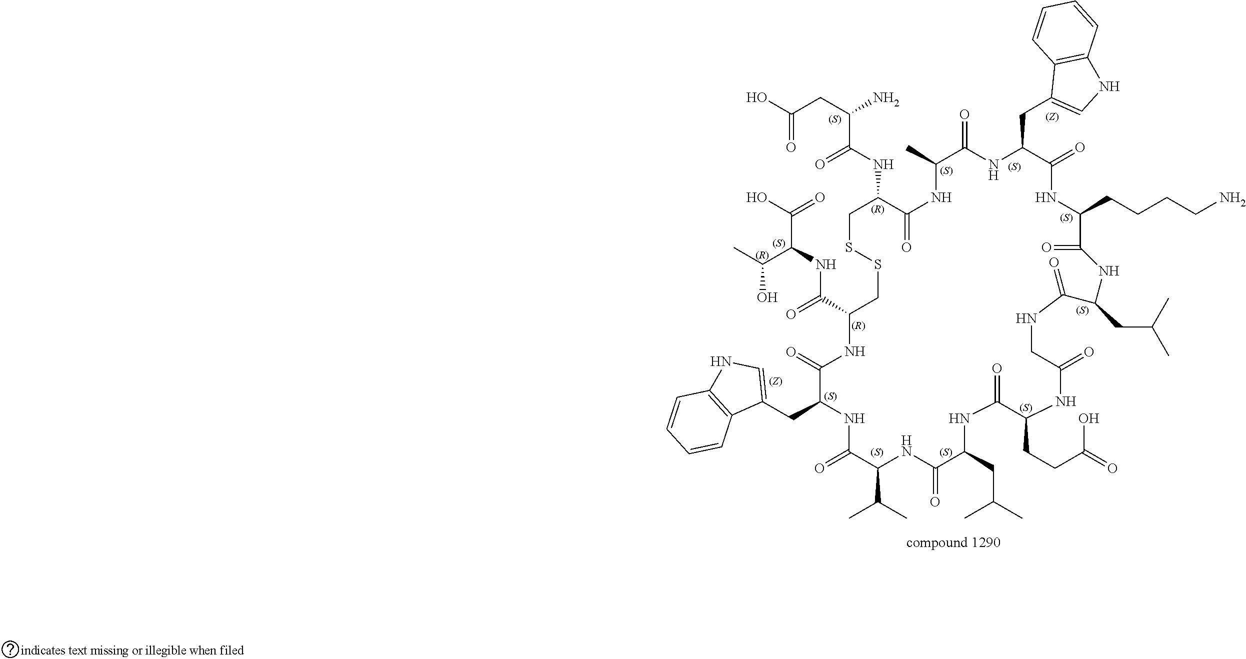

### Preparation of Compound 1290:

Peptide was synthesized using standard Fmoc chemistry (CTC resin).

Resin preparation: To the vessel containing CTC resin (1.00 g, 1.00 mmol, 1.00 mmol/g) and Fmoc-Thr(tBu)-OH (397.0 mg, 1.00 mmol, 1.00 equiv.) in DCM (10 mL) was added DIEA (4.00 equiv.) dropwise and mixed for 2 h with N2 bubbling at 25° C. Then MeOH (1.0 mL) was added and bubbled with N2 for another 30 min. The resin was washed with DMF (20 mL), followed by the addition of 20% piperidine in DMF (10 mL) and bubbled with N2 for 30 min at 25° C. for Fmoc deprotection. The mixture was filtered and the resin was washed with DMF (10 mL) before proceeding to next step.

Coupling: A solution of Fmoc-Cys(Trt)-OH (1.76 g, 3.0 mmol, 3.00 equiv.), HBTU (0.82 g, 2.86 mmol, 2.85 equiv.) in DMF (10 mL) was added to the resin with N₂ bubbling. Then DIEA (6.00 equiv.) was added to the mixture dropwise and bubbled with N₂ for 30 min at 25° C. The coupling reaction was monitored by ninhydrin test, if it showed colorless, the coupling was completed. The resin was then washed with DMF (20 mL).

Deprotection: 20% piperidine in DMF (20 mL) was added to the resin and the mixture was bubbled with N2 for 30 min at 25° C. The deprotection reaction was monitored by ninhydrin test, if it showed blue or brownish red, the reaction was completed. The resin was then washed with DMF (20 mL).

Steps 2 and 3 were repeated for the following amino acids elongation: Number # 3-13, Table 1.

After all the steps were completed, the resin was washed with DMF (50 mL), MeOH (50 mL), then dried under reduced pressure to afford resin-bound peptide intermediate 1 (CTC resin, 2.40 g, 1.00 mmol).

TABLE 2
Table 2: The list of amino acids and the corresponding reagents used on SPPS.

| # | Materials | Coupling reagents |
|---|---|---|
| 1 | Fmoc-Thr(tBu)-OH (1.00 equiv.) | DIEA (4.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-Lys(Boc)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |

### Peptide Cleavage and Cyclization:

Cleavage: A solution of TFA/TIS/H2O/3-mercaptopropanoic acid (92.5/2.5/2.5/2.5, v/v/v, 40 mL) was added to the resin (intermediate 1, 0.50 mmol, another 0.5 mmol was used for compound 1291) above at room temperature and stirred for 2 h. After filtration, After filtration, the filtrate was collected and precipitated with cold isopropyl ether (200 mL), then filtered off, and the solid was washed with isopropyl ether (100 mL) twice, and the crude peptide was dried under reduced pressure for 2 h to afford intermediate 2 (0.50 mmol, crude) as a white solid.

Cyclization: To the crude peptide (intermediate 2) in MeCN/H₂O (1/1, v/v, 500 mL) was added 0.1 M l₂/AcOH dropwise until a yellow color persisted, then the mixture was stirred at 25° C. for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford compound 1290 (93.0 mg, 94.6% purity, 15.5% yield) as a white solid. LCMS: RT=0.81 min, MS calcd.: Mₐᵥ=1521.76, mass observed: [M+H]⁺=1522.70, [M+2H]²⁺=761.50.

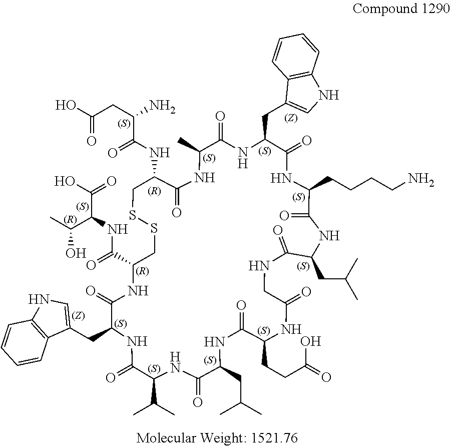

### Example 2. Procedure for Preparation of Compound 1291

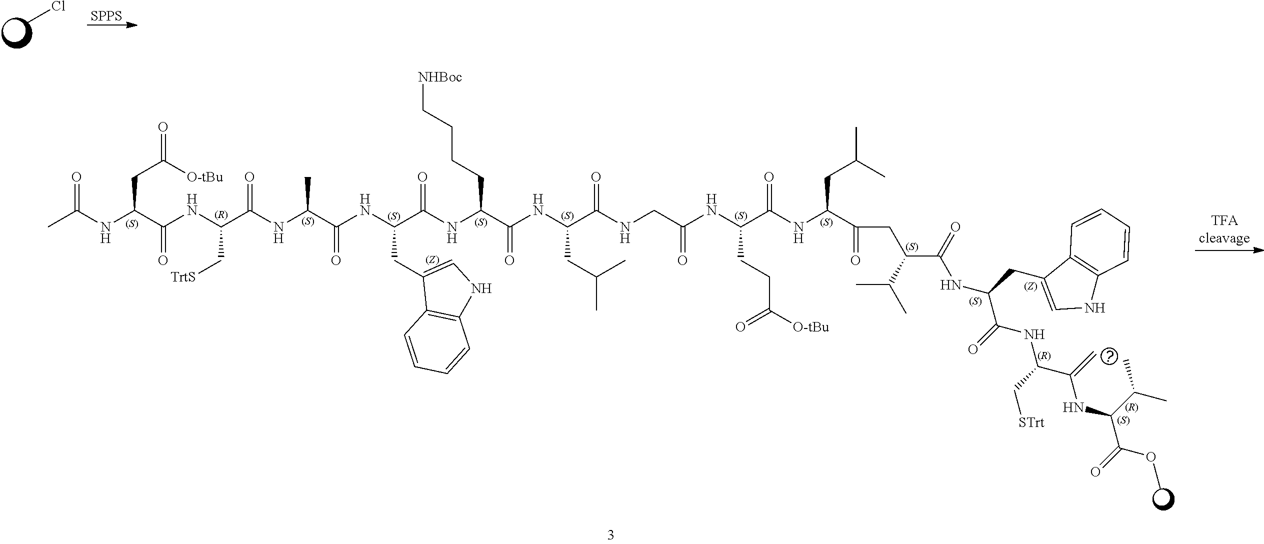

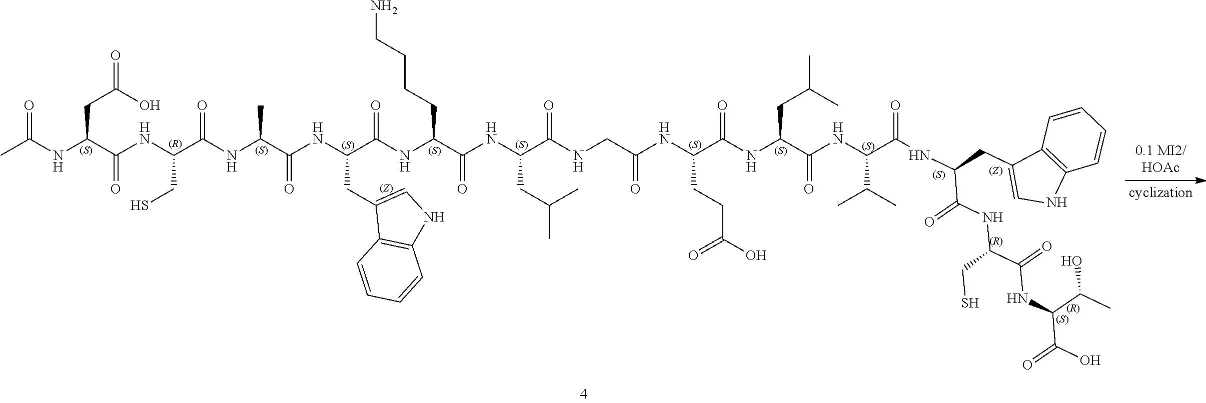

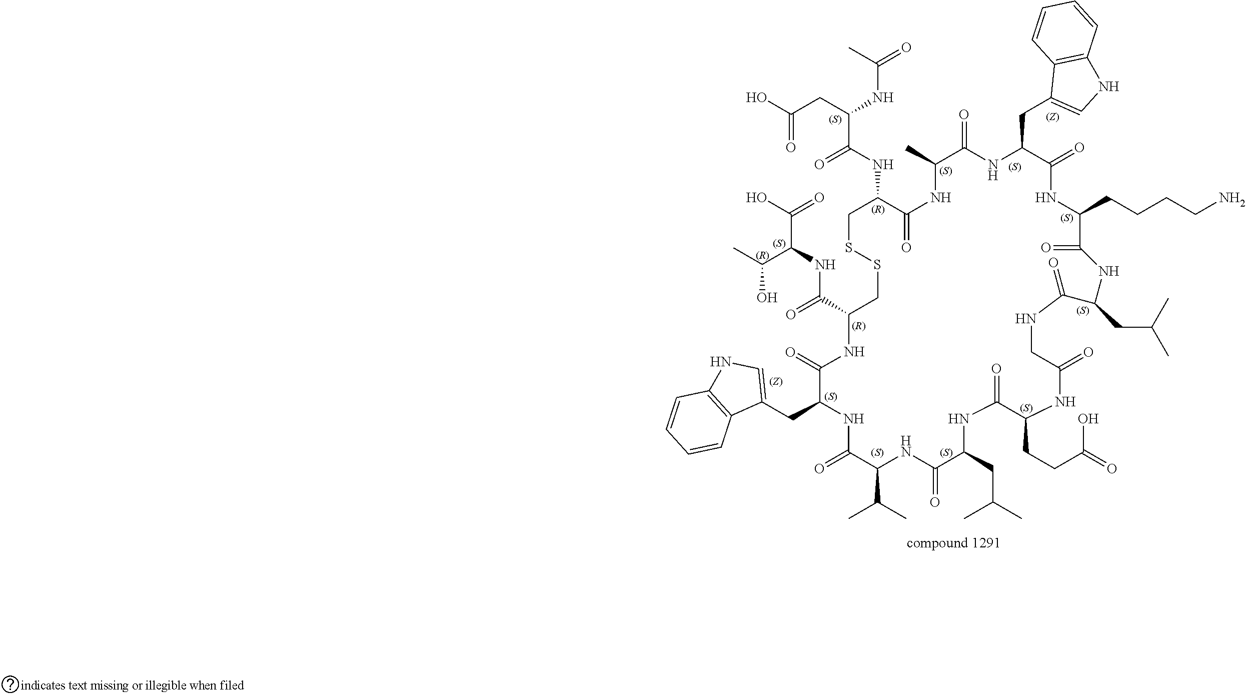

### Preparation of Compound 1291:

Intermediate 3 (peptide resin) was synthesized by performing acetylation on peptide resin (intermediate 1, 0.50 mmol).

Acetylation: A solution of Ac20/NMM/DMF (10/5/85, v/v/v, 40 ml) was added to the resin, the mixture was bubbled with N2 for 20 min. The acetylation reaction was monitored by ninhydrin test. The resin was then washed with DMF (20 mL) , MeOH (20 mL), then dried under reduced pressure to afford resin-bound peptide intermediate 4 (CTC resin, 1.23 g, 0.50 mmol).

TABLE 3
Acetylation on SPPS.

| # | Materials | Coupling reagents |
|---|---|---|
| 1 | Acetylation | Ac2O/NMM/DMF (10/5/85, v/v/v, 20 mL) |

Peptide cleavage and cyclization were performed by following the procedure mentioned in the peptide cleavage and cyclization reaction in Example 1. 0.50 mmol resin afforded compound 1291 (148.0 mg, 95.5% purity, 18.1% yield) as a white solid. LCMS: RT=1.53 min, MS calcd .: Mₐᵥ=1563.79, mass observed: [M+H]⁺=1564.40, [M+2H]²⁺=782.80.

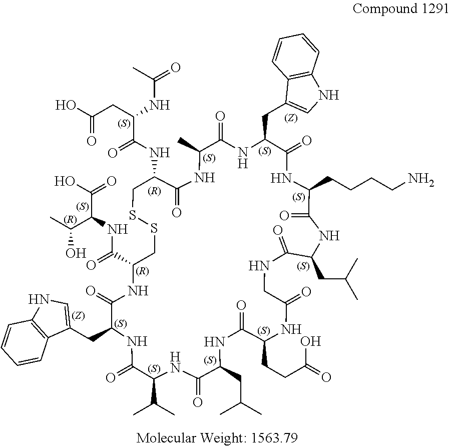

### Example 3. Procedure for Preparation of Compound 1292

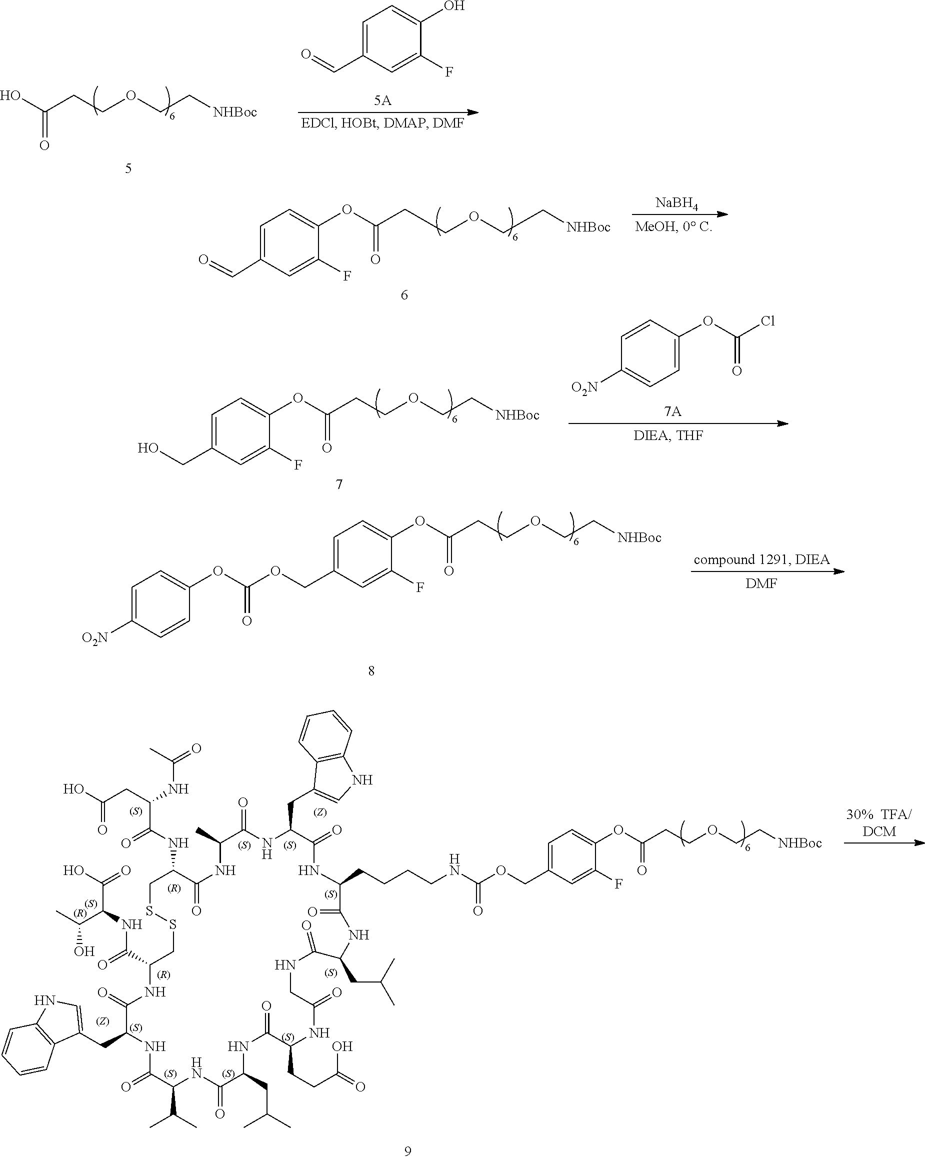

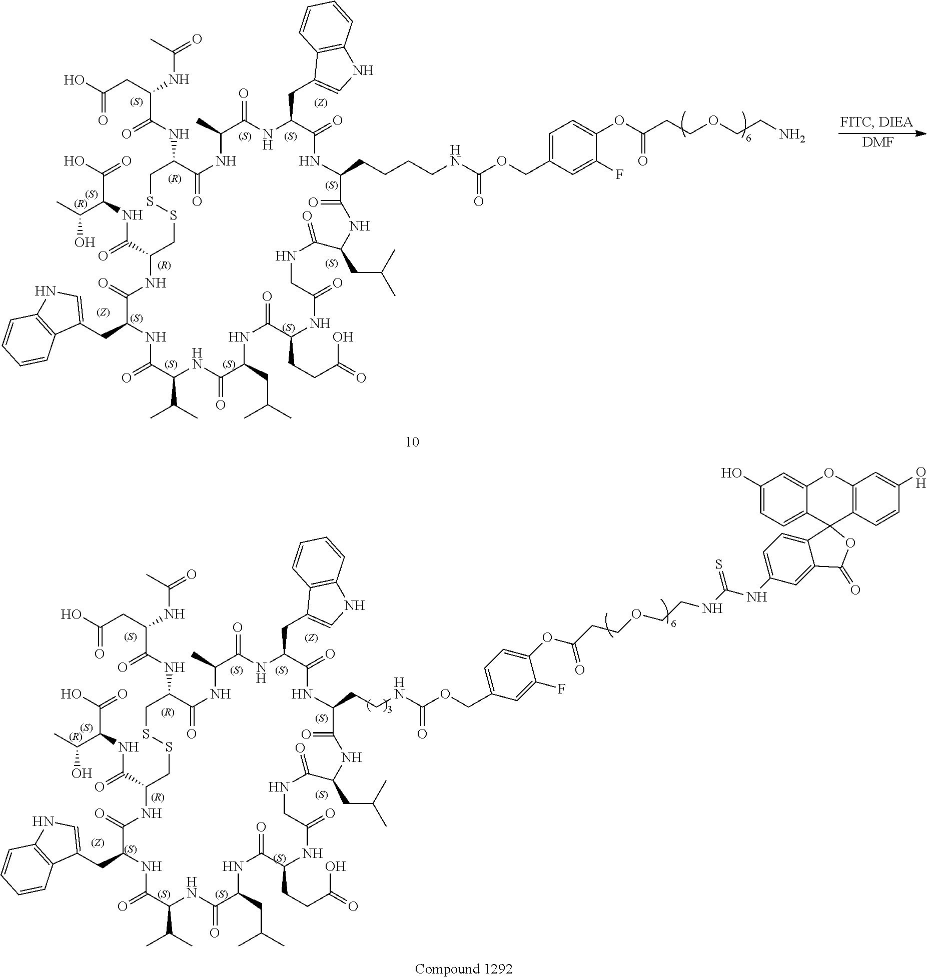

### Preparation of Intermediate 6

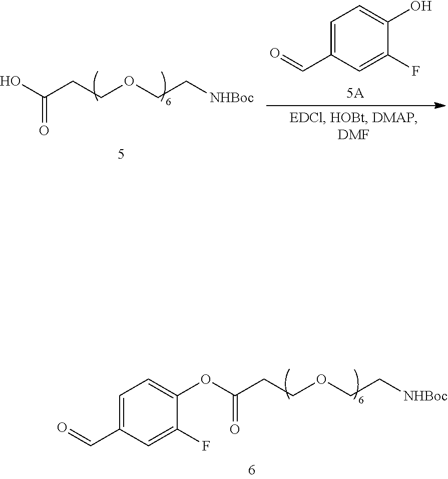

A mixture of intermediate 5 (1.40 g, 3.09 mmol, 1.00 equiv.) and intermediate 5A (1.30 g, 9.26 mmol, 3.00 equiv.), HOBt (1.25 g, 9.26 mmol, 3.00 equiv.), DMAP (188.56 mg, 1.54 mmol, 0.50 equiv.) and EDCI (1.78 g, 9.26 mmol, 3.00 equiv.) in DMF (0.2 mL) was stirred at 25° C. for 16 h. The mixture was purified by Flash (C18, A: 0.075% TFA/H₂O, B: MeCN) directly, followed by lyophilization to afford intermediate 6 (1.10 g, 1.91 mmol, 61.9% yield) as yellow oil.

### Preparation of Intermediate 7

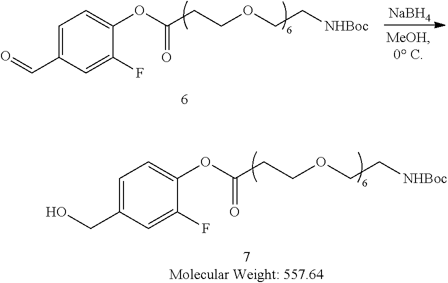

A mixture of intermediate 6 (1.85 g, 3.21 mmol, 1.00 equiv.) was dissolved in MeOH (5 mL), to the reaction mixture was added a mixture of NaBH₄ (145.91 mg, 3.86 mmol, 1.20 equiv.) in MeOH (1 mL) dropwise at 0° C. The reaction mixture was stirred at 0° C.for 2 h. After completion the reaction was monitored by LC-MS, the mixture was acidified by 1 M HCl to pH=5, then purified by prep-HPLC (A: 0.075% TFA/H₂O, B: MeCN) directly, followed by lyophilization to afford intermediate 7 (1.35 g, 2.34 mmol, 72.7% yield) as colorless oil. LCMS: RT=9.3 min, MS calcd.: Mₐᵥ=577.64, mass observed: [M+H]⁺⁼578.30, [M+H₂O+H]⁺=595.4, [M−Boc+H]⁺=478.37.

### Preparation of Intermediate 8:

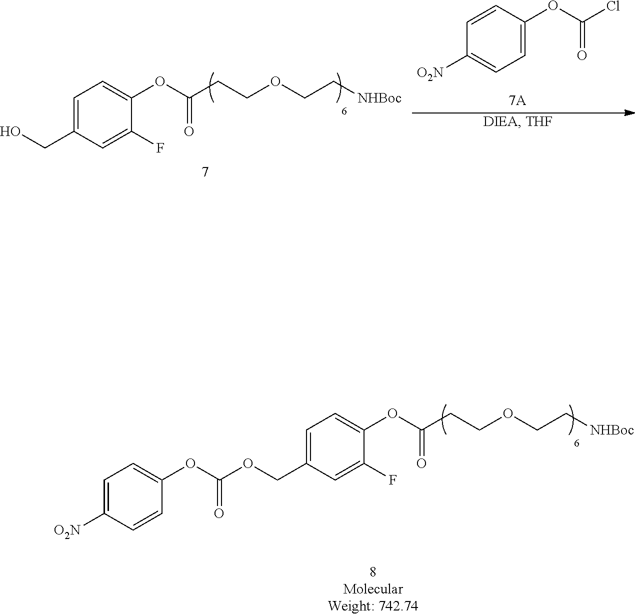

To a solution of intermediate 7 (1.20 g, 2.08 mmol, 1.00 equiv.), TEA (420.43 mg, 4.15 mmol, 578.30 μL, 2.00 equiv.) in DCM (10 mL) was added intermediate 7A (460.61 mg, 2.29 mmol, 1.10 equiv.) The reaction was stirred at 25° C. for 4 h. After completion monitored by LC-MS, the mixture was purified by prep-HPLC (A: 0.075% TFA/H₂O, B: MeCN) directly, followed by lyophilization to afford intermediate 8 (900.0 mg, 1.21 mmol, 58.3% yield) as a yellow oil. LCMS: RT=9.3 min, MS calcd.: Mₐᵥ=742.74, mass observed: [M+H]⁺=743.2, [M+H₂O+H]⁺=761.3, [M−Boc+H]⁺=643.3.

### Preparation of Intermediate 9:

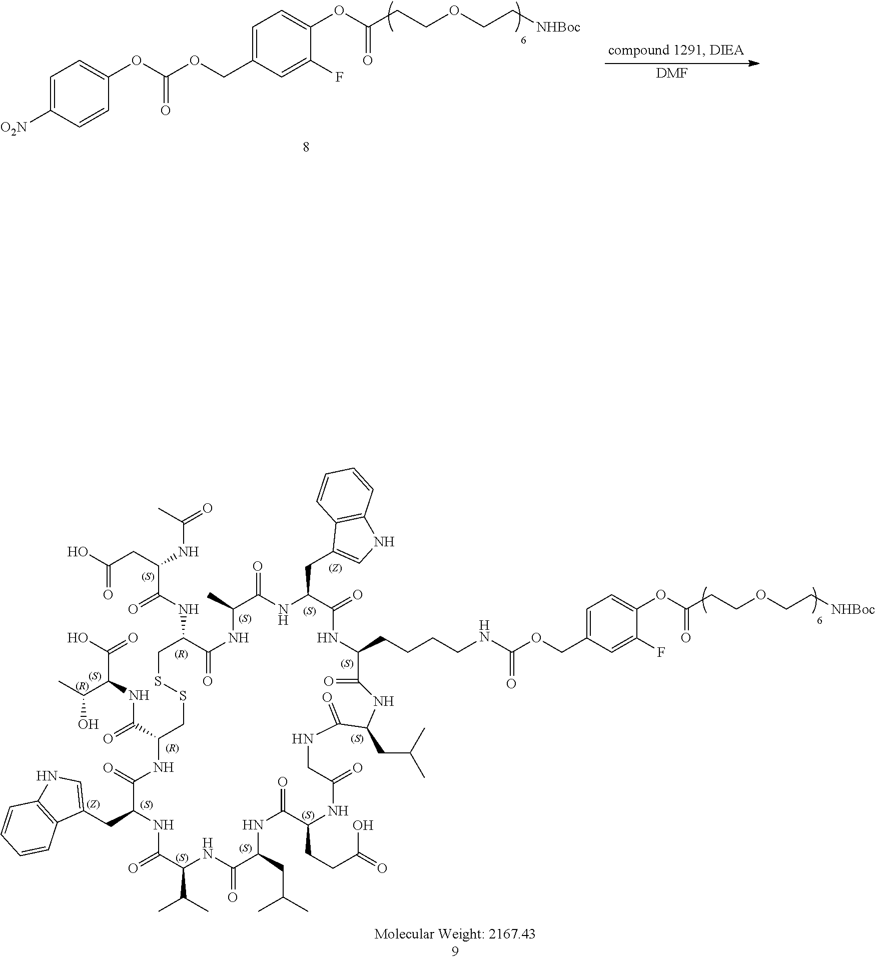

A mixture of intermediate 8 (500.00 mg, 667.50 μmol, 2.00 equiv.) and compound 1291 (521.5 mg, 333.70 μmol, 1.00 equiv.), DIEA (129.0 mg, 174.3 μL, 1.00 mmol, 3.00 equiv.) in DMF (5 mL) was stirred at 25° C.for 2 h. After completion monitored by LC-MS, the mixture was acidified by 1 M HCl to PH=5, then purified by prep-HPLC (A: 0.075% TFA/H₂O, B: MeCN) directly, followed by lyophilization to afford intermediate 9 (517.3 mg, 238.69 μmol, 81.2% purity, 71.5% yield) as a white solid. LCMS: RT=1.05 min, MS calcd.: Mₐᵥ=2167.43, mass observed: [M−Boc+2H]²⁺=1034.58.

### Preparation of Intermediate 10:

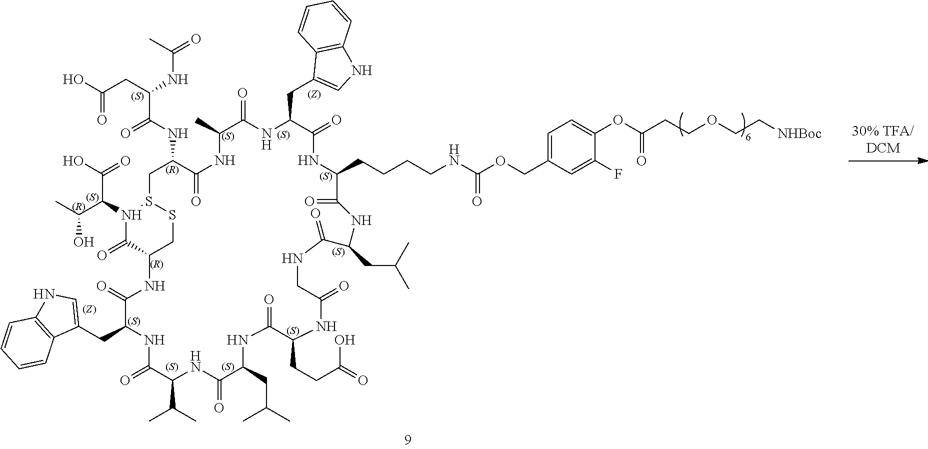

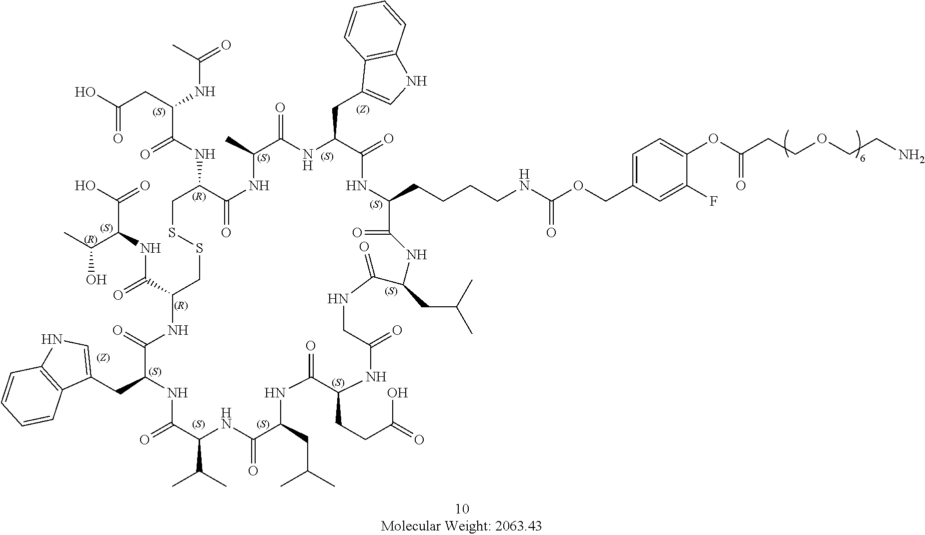

A mixture of intermediate 9 (517.3 mg, 238.69 μmol) in TFA/DCM (3/7, v/v, 3 mL) was stirred at 0° C. for 2 h. After completion monitored by LC-MS, the solvent was removed under reduce pressure. The residue was purified by prep-HPLC (A: 0.075% TFA/H₂O, B: MeCN), followed by lyophilization to afford intermediate 10 (429.27 mg, 207.65 μmol, 87.0% yield, TFA salt) as a white solid. UPLC: RT=0.92 min, MS calcd.: Mₐᵥ=2067.43, mass observed: [M+2H]²⁺=1034.12.

### Preparation of Compound 1292:

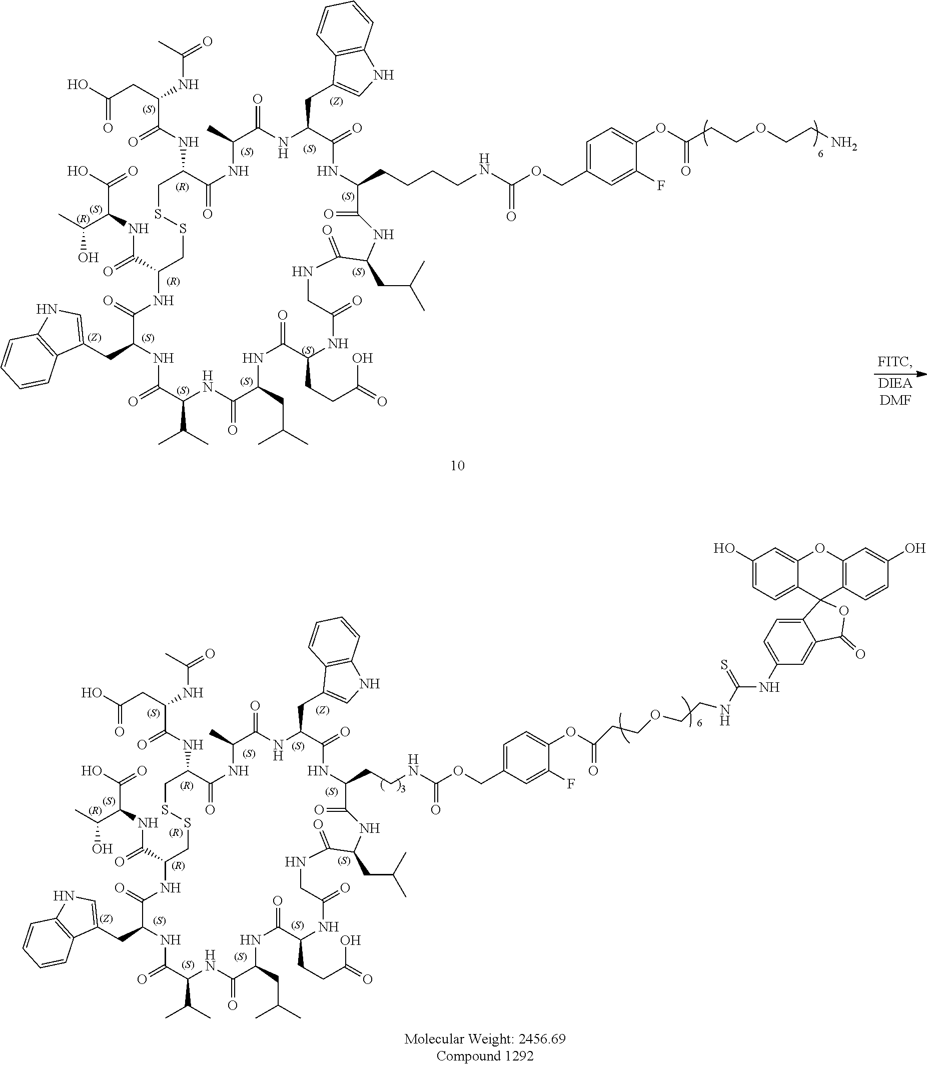

To a mixture of intermediate 10 (429.27 mg, 207.65 μmol, 1.00 equiv.) and FITC (121.28 mg, 311.47 μmol, 1.50 equiv.) in DMF (0.2 mL) was added DIEA (20.76 mg, 934.42 μmol, 162.75 μL, 4.50 equiv.) at 25° C. The mixture was stirred at 25° C.for 2 h. After completion monitored by LC-MS, the mixture was acidified by 1 M HCl to pH=5, then purified by prep-HPLC (A: 0.075% TFA/H₂O, B: MeCN) directly, followed by lyophilization to afford Compound 1292 (115.0 mg, 92.0% purity, 22.5% yield) as a yellow solid. LCMS: RT=2.08 min, MS calcd.: Mₐᵥ=2456.69, mass observed: [M+2H]²⁺=1229.1, [M+3H]³⁺=819.7, [2M+3H]³⁺=1637.9.

### Example 4. Procedure for Preparation of Compound 1294.

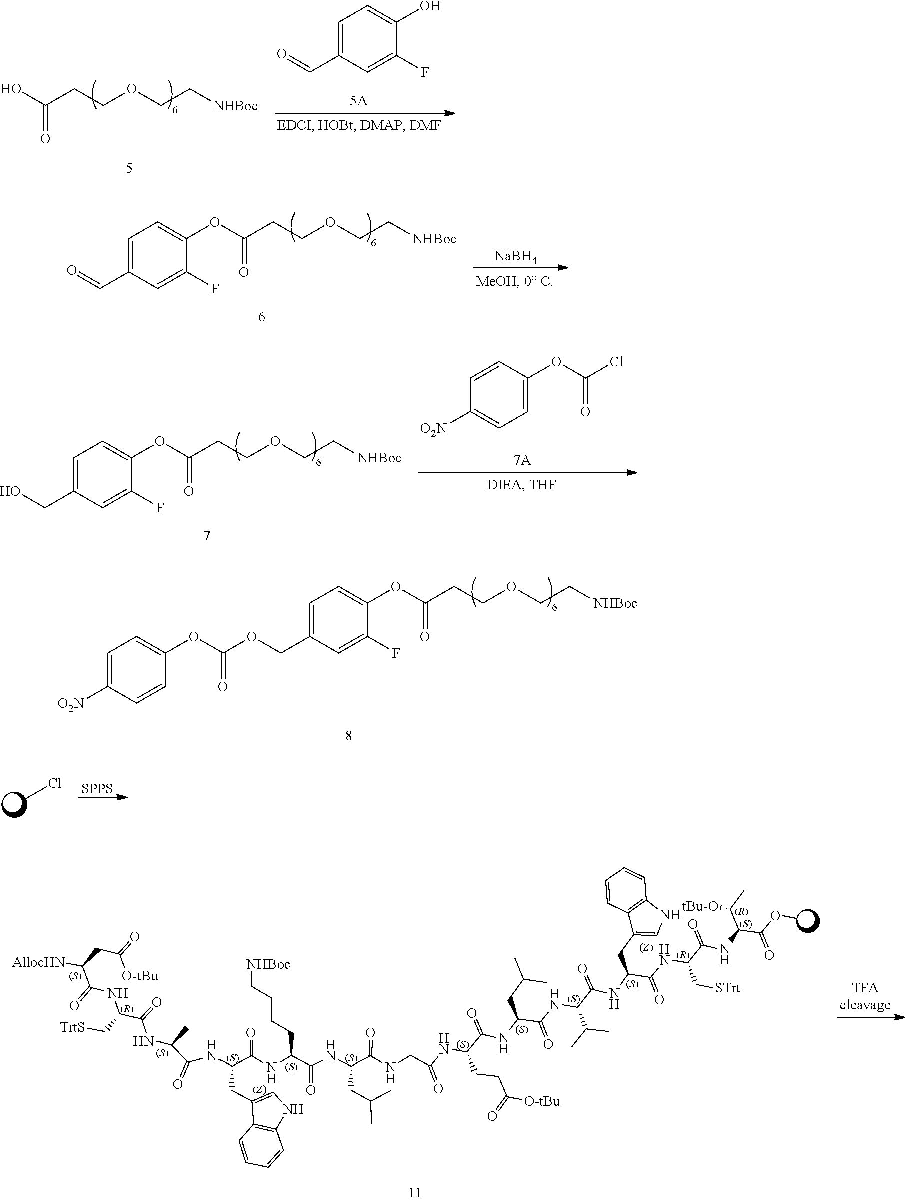

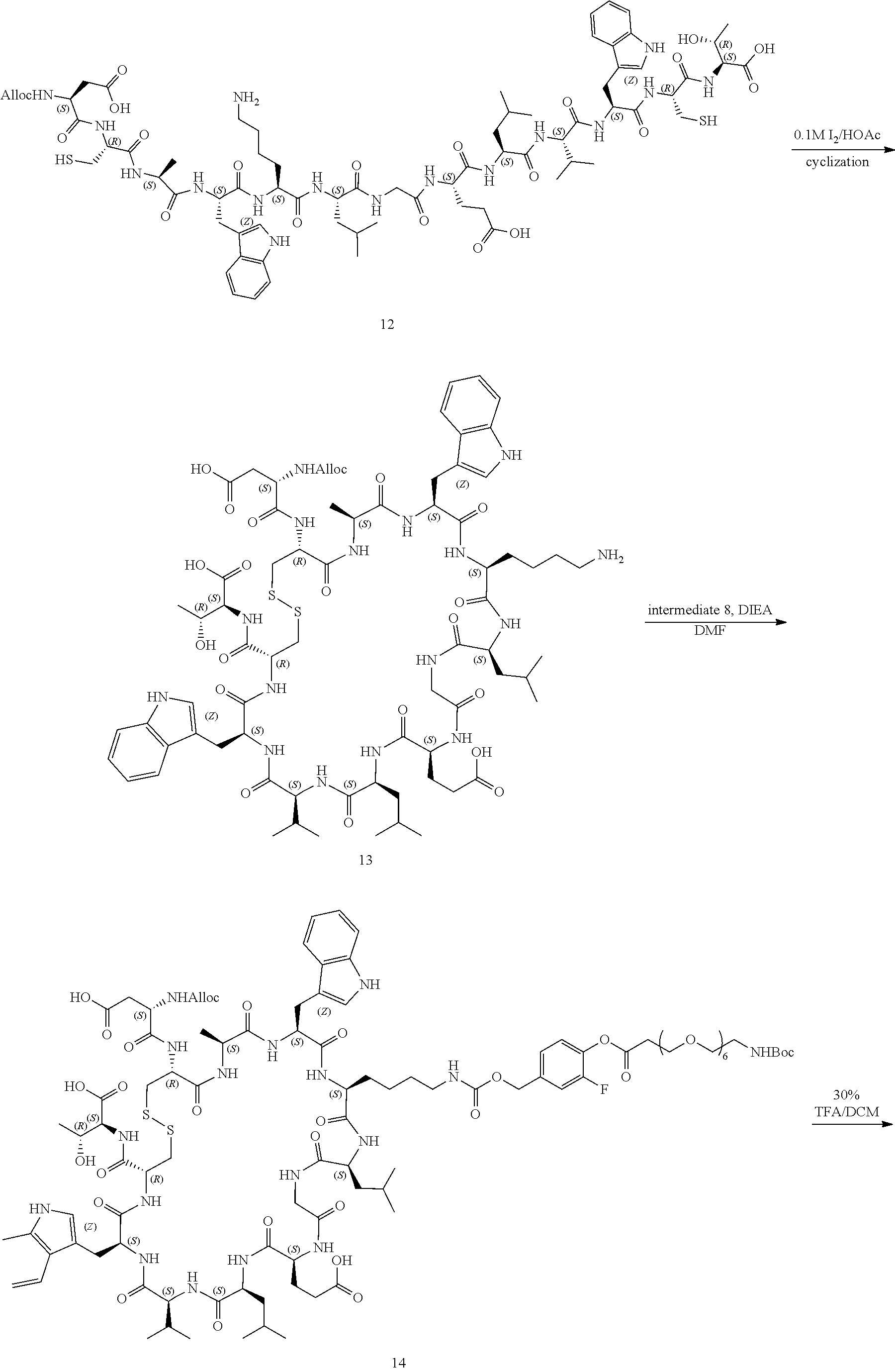

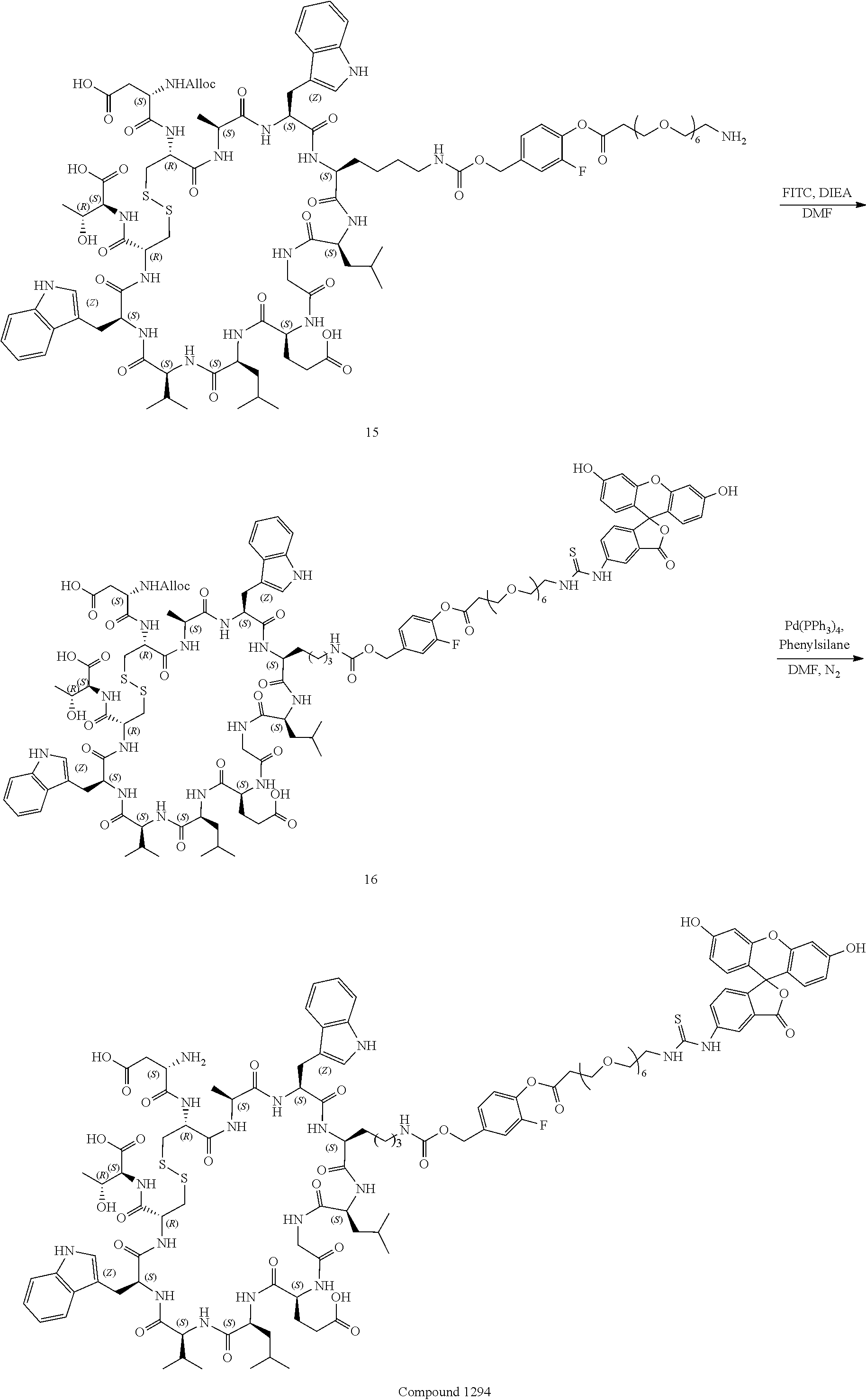

### Preparation of Intermediate 6, 7, 8:

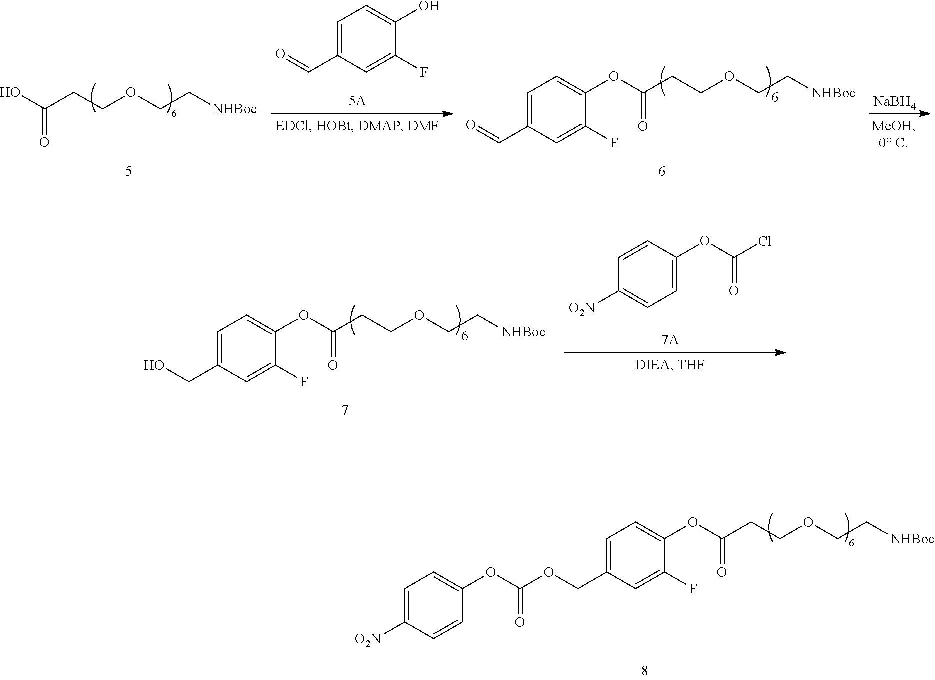

Intermediate 6, 7, 8 were synthesized by following the procedure mentioned in the preparation of intermediates 6, 7, 8 in Example 3.

### Preparation of Intermediate 13:

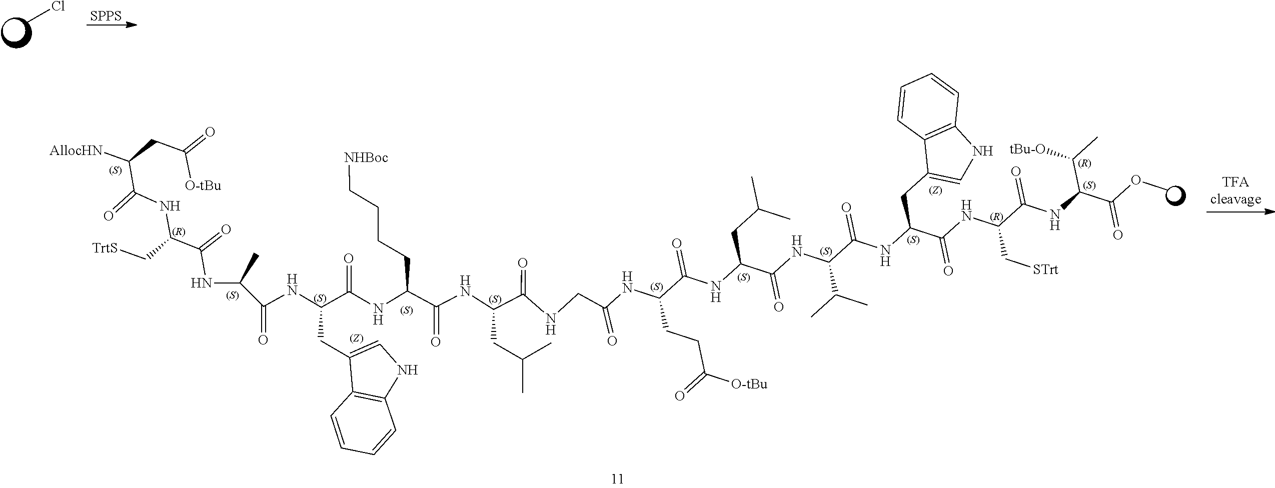

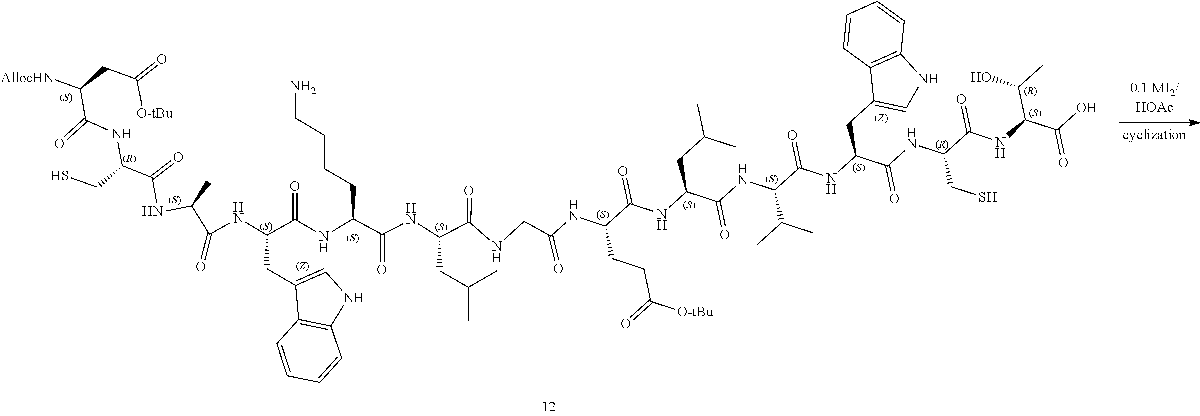

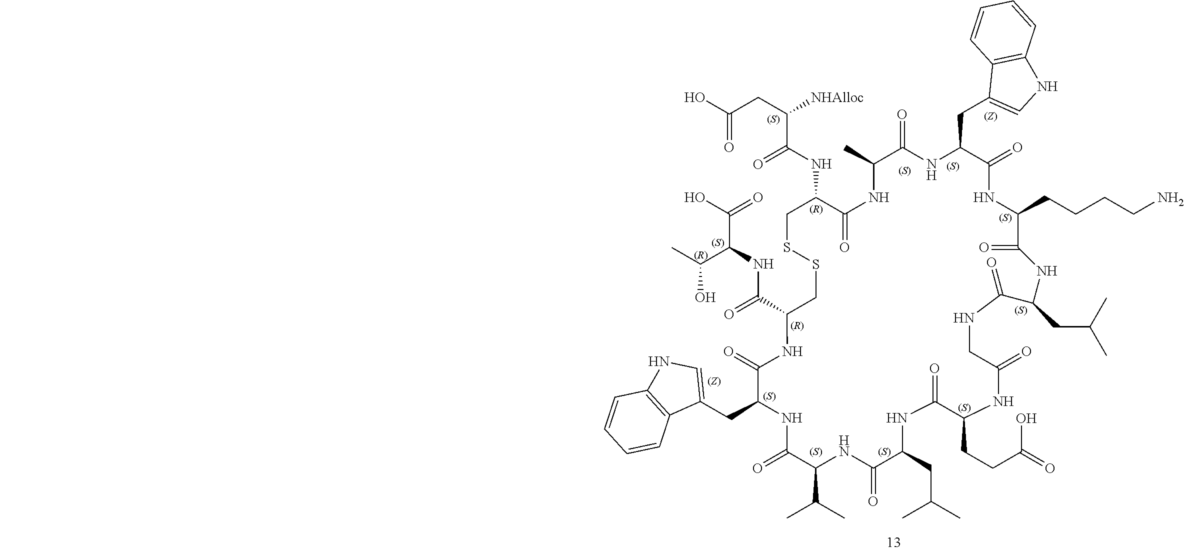

Peptide was synthesized using standard Fmoc chemistry (CTC resin).

Resin preparation: To the vessel containing CTC resin (1.00 g, 1.00 mmol, 1.00 mmol/g) and Fmoc-Thr(tBu)-OH (397.0 mg, 1.00 mmol, 1.00 equiv.) in DCM (10 mL) was added DIEA (4.00 equiv.) dropwise and mixed for 2 h with N2 bubbling at 25° C. Then MeOH (1.0 mL) was added and bubbled with N2 for another 30 min. The resin was washed with DMF (20 mL) , followed by the addition of 20% piperidine in DMF (10 mL) and bubbled with N2 for 30 min at 25° C.for Fmoc deprotection. The mixture was filtered and the resin was washed with DMF (10 mL) before proceeding to next step.

Coupling: A solution of Fmoc-Cys(Trt)-OH (1.76 g, 3.0 mmol, 3.00 equiv.), HBTU (0.82 g, 2.86 mmol, 2.85 equiv.) in DMF (10 mL) was added to the resin with N₂ bubbling. Then DIEA (6.00 equiv.) was added to the mixture dropwise and bubbled with N₂ for 30 min at 25° C. The coupling reaction was monitored by ninhydrin test, if it showed colorless, the coupling was completed. The resin was then washed with DMF (20 mL).

Deprotection: 20% piperidine in DMF (20 mL) was added to the resin and the mixture was bubbled with N2 for 30 min at 25° C. The deprotection reaction was monitored by ninhydrin test, if it showed blue or brownish red, the reaction was completed. The resin was then washed with DMF (20 mL).

Steps 2 and 3 were repeated for the following amino acids elongation: Number # 3-13, Table 3.

Alloc-CI couplig on N-terminal: the resin was washed with DCM (20 mL) . A solution of Allo-CI (0.72 g, 6.0 mmol, 6.00 equiv.) in DCM (10 mL) was added to the resin with N₂ bubbling. Then DIEA (12.00 equiv.) was added to the mixture dropwise and bubbled with N₂ for 30 min at 25° C. The coupling reaction was monitored by ninhydrin test, if it showed colorless, the coupling was completed. The resin was then washed with DCM (50 mL)*3, DMF (50 mL)*3, MeOH (50 mL)*3, then dried under reduced pressure to afford resin-bound peptide intermediate 11 (CTC resin, 2.35 g, 1.00 mmol).

TABLE 4
The list of amino acids and the corresponding reagents used on SPPS.

| # | Materials | Coupling reagents |
|---|---|---|
| 1 | Fmoc-Thr(tBu)-OH (1.00 equiv.) | DIEA (4.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Fmoc-Lys(Dde)-OH (3.00 equiv.) | HBTU (2.85 equiv.) and DIEA (6.00 equiv.) |
| 10 | Fmoc-Trp-OH (3.00 equiv.) | HBTU (2.85 equiv.) and DIEA (6.00 equiv.) |
| 11 | Fmoc-Ala-OH (3.00 equiv.) | HBTU (2.85 equiv.) and DIEA (6.00 equiv.) |
| 12 | Fmoc-Cys(Trt)-OH (3.00 equiv.) | HBTU (2.85 equiv.) and DIEA (6.00 equiv.) |
| 13 | Fmoc-Asp(OtBu)-OH (3.00 equiv.) | HBTU (2.85 equiv.) and DIEA (6.00 equiv.) |
| 14 | Alloc-Cl (6.00 equiv.) | DIEA (12.00 equiv.) |

### Peptide Cleavage and Cyclization:

Cleavage: A solution of TFA/TIS/H₂O/ 3-mercaptopropanoic acid (92.5/2.5/2.5/2.5, v/v/v, 40 mL) was added to the resin (intermediate 11, 1.00 mmol) above at room temperature and stirred for 2 h.

After filtration, the filtrate was collected and precipitated with cold isopropyl ether (400 mL), then filtered off, and the solid was washed with isopropyl ether (200 mL) twice, and the crude peptide was dried under reduced pressure for 2 h to afford intermediate 12 (1.00 mmol, crude) as a white solid.

Cyclization: To the crude peptide (intermediate 12) in MeCN/H₂O (1/1, v/v, 1000 mL) was added 0.1 M I₂/AcOH dropwise until a yellow color persisted, then the mixture was stirred at 25° C.for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford intermediate 13 (280.5 mg, 17.4% yield) as a white solid. LCMS: RT=7.9 min, MS calcd.: Mₐᵥ=1605.83, mass observed: [M+H]⁺=1606.80, [M+2H]²⁺=803.52.

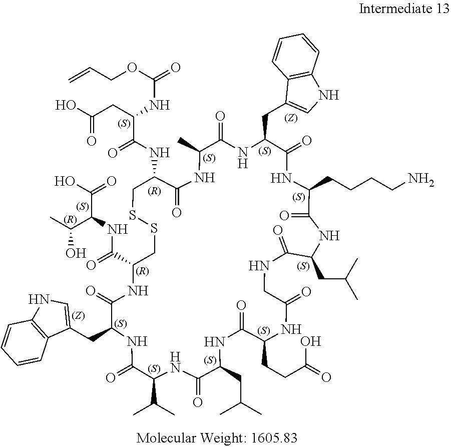

### Preparation of Intermediate 14:

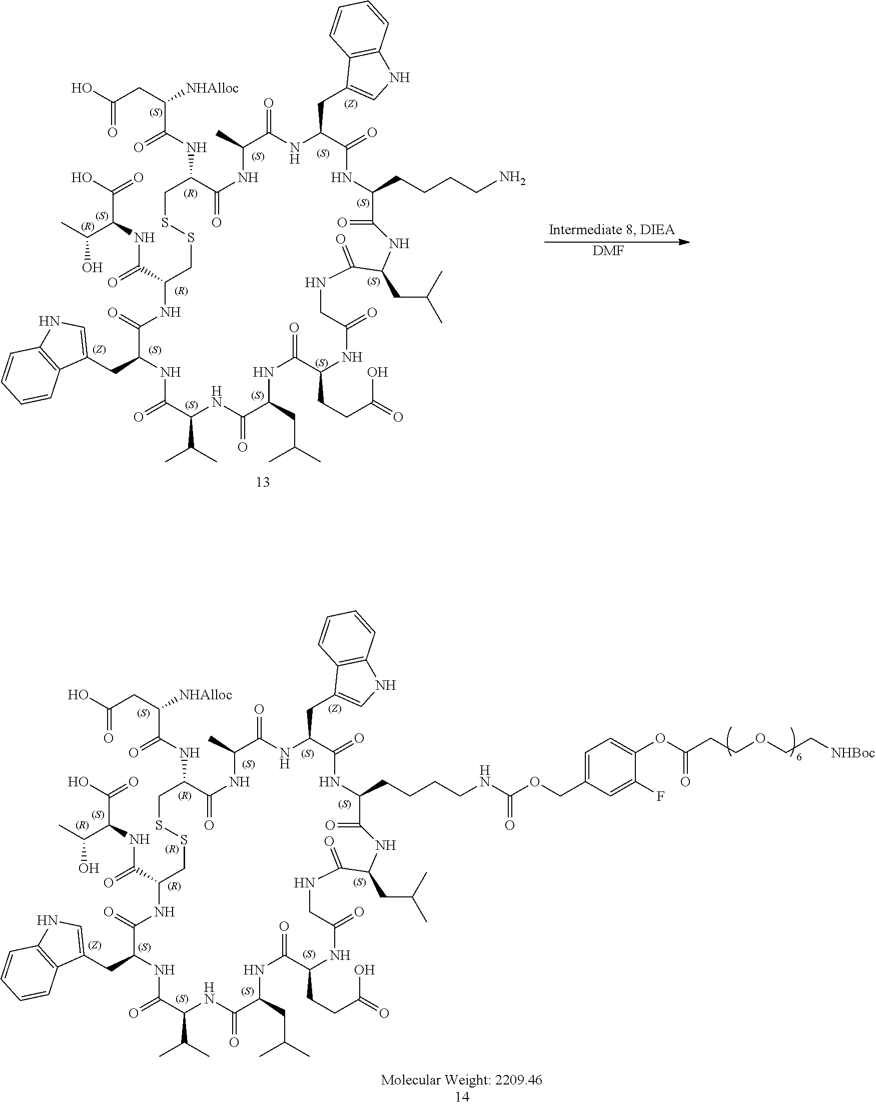

A mixture of intermediate 8 (475.28 mg, 0.63 mmol, 2.00 equiv.) and intermediate 13 (500.0 mg, 0.31 mmol, 1.00 equiv.), DIEA (120.5 mg, 162.9 μL, 0.93 mmol, 3.00 equiv.) in DMF (5 mL) was stirred at 25° C. for 2 h. After completion monitored by LC-MS, the mixture was acidified by 1 M HCl to pH=5, then purified by prep-HPLC (A: 0.075% TFA/H₂O, B: MeCN) directly, followed by lyophilization to afford intermediate 14 (517.3 mg, 74.6% yield) as a white solid. LCMS: RT=0.960 min, MS calcd.: Mₐᵥ=2209.46, mass observed: [M−Boc+2H]²⁺=1055.28.

### Preparation of Intermediate 15:

A mixture of intermediate 14 (517.3 mg) in TFA/DCM (3/7, 5 mL) was stirred at 0° C.for 2 h. After completion monitored by LC-MS, the mixture was acidified by 1 M HCl to PH=5, then purified by prep-HPLC (A: 0.075% TFA/H₂O, B: MeCN) directly, followed by lyophilization to afford compound 1579 (437.0 mg, 97.9% purity, 88.4% yield, TFA salt) as a white solid.

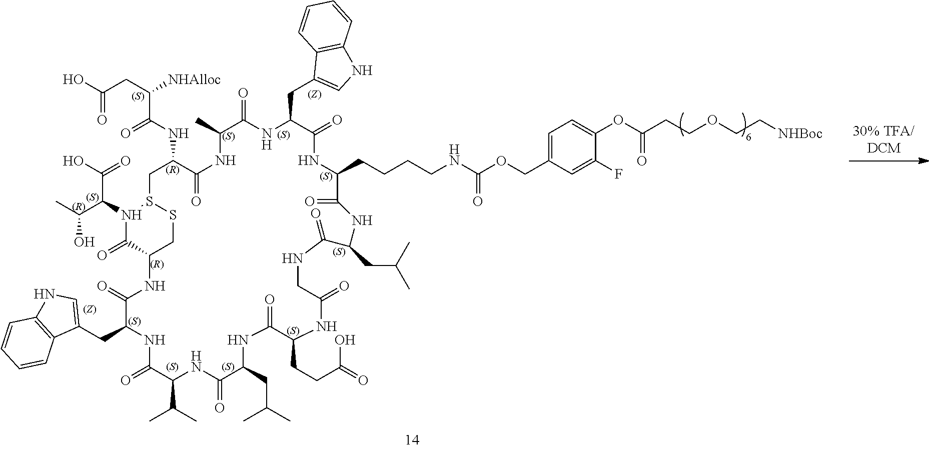

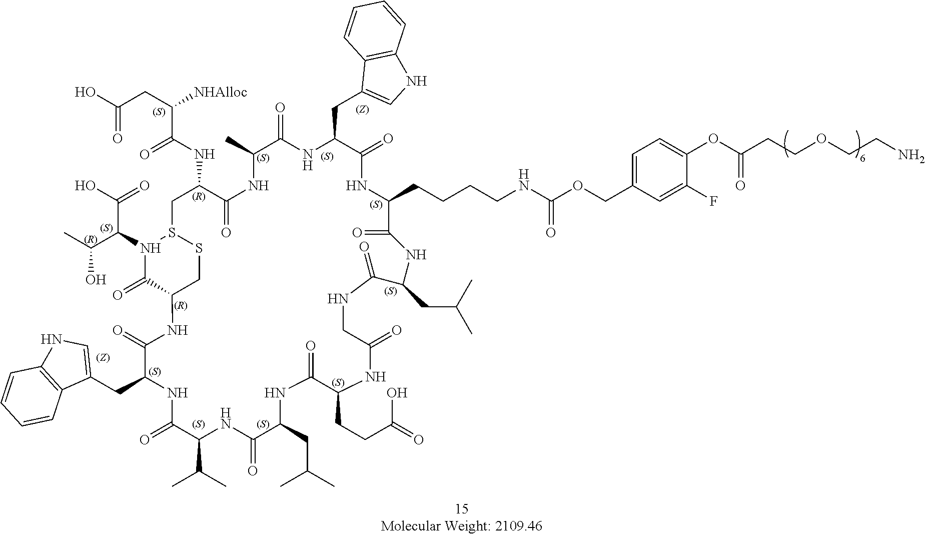

### Preparation of Intermediate 16:

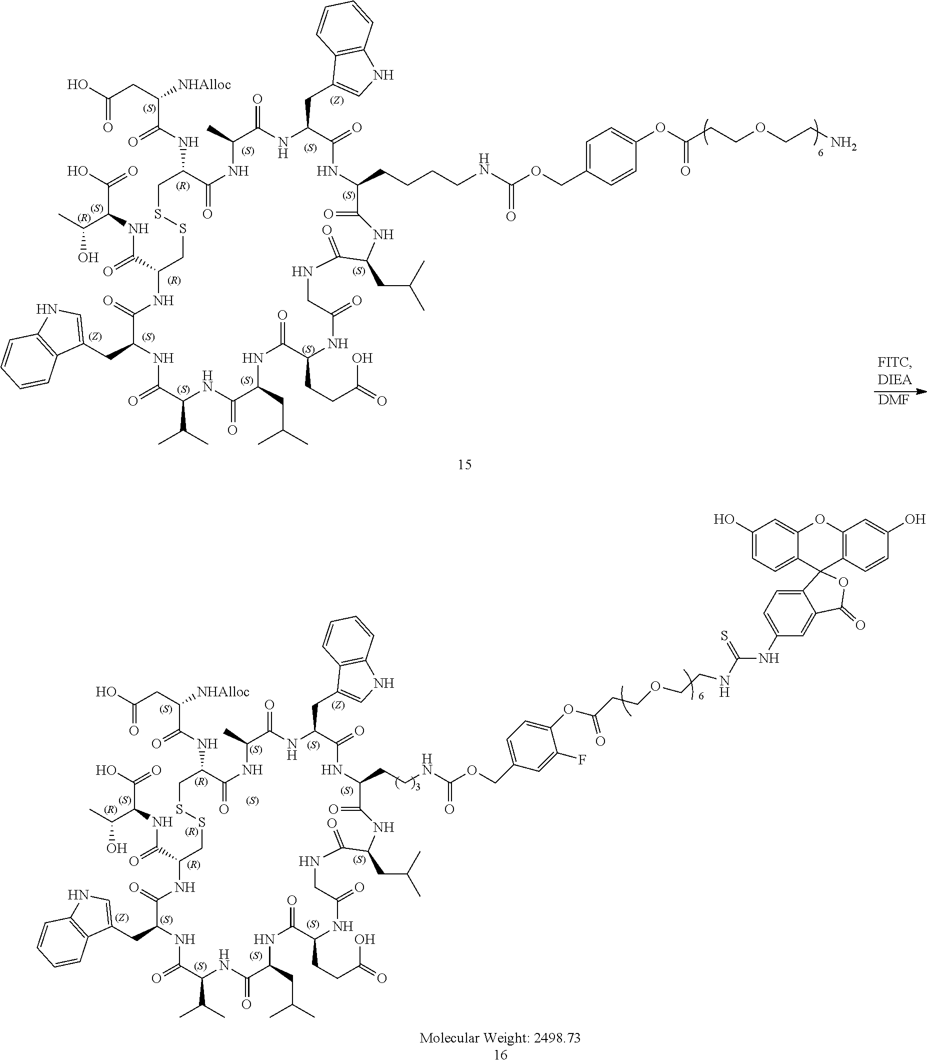

To a mixture of intermediate 15 (437.0 mg, 207.2 μmol, 1.00 equiv.) and FITC (120.8 mg, 310.80 μmol, 1.50 equiv.) in DMF (0.2 mL) was added DIEA (120.7 mg, 162.5 μL, 932.4 μmol, 4.50 equiv.) at 25° C. The mixture was stirred at 25° C.for 2 h. After completion monitored by LC-MS, the mixture was acidified by 1 M HCl to pH=5, then purified by prep-HPLC (A: 0.075% TFA/H₂O, B: MeCN) directly, followed by lyophilization to afford intermediate 16 (300.0 mg, 92.5% purity, 57.9% yield) as a yellow solid. LCMS: RT=9.3 min, MS calcd.: Mₐᵥ=2498.73, mass observed: [M+2H]²⁺=1249.87.

### Preparation of Compound 1294:

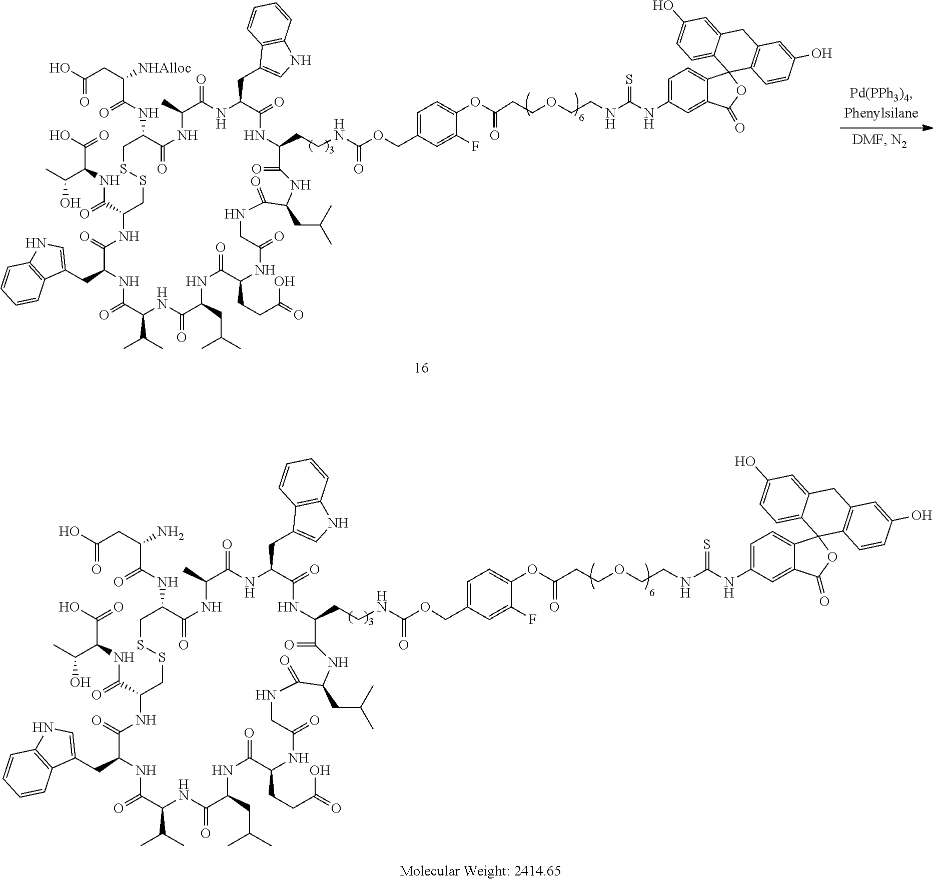

### Compound 1294

To a mixture of intermediate 16 (300.0 mg, 120.06 μmol, 1.00 equiv.) dissolved in DMF (3 mL) was added Pd(PPh₃)₄ (20.81 mg, 18.01 μmol, 0.15 equiv.) and phenylsilane (129.92 mg, 1.20 mmol, 148.14 μL, 10.00 equiv.). The mixture was stirred at 20° C.for 1 h. and the resulting reaction was stirred for 5 min at 0° C. After completion monitored by LC-MS. The mixture was acidified by 1 M HCl to pH=5, then purified by prep-HPLC (A: 0.075% TFA/H₂O, B: MeCN) directly, followed by lyophilization to afford Compound 1294 (98.3 mg, 87.0% purity, 29.5% yield) as a yellow solid. LCMS: RT=2.02 min, MS calcd.: Mₐᵥ=2414.65, mass observed: [2M +3H]³⁺=1610.6, [M+2Na]²⁺=1230.5, [M+H+Na]²⁺=1219.3, [M+2H]²⁺=1208.1, [M+3H]³⁺=805.8.

### Example 5. Procedure for Preparation of Compound 1295.

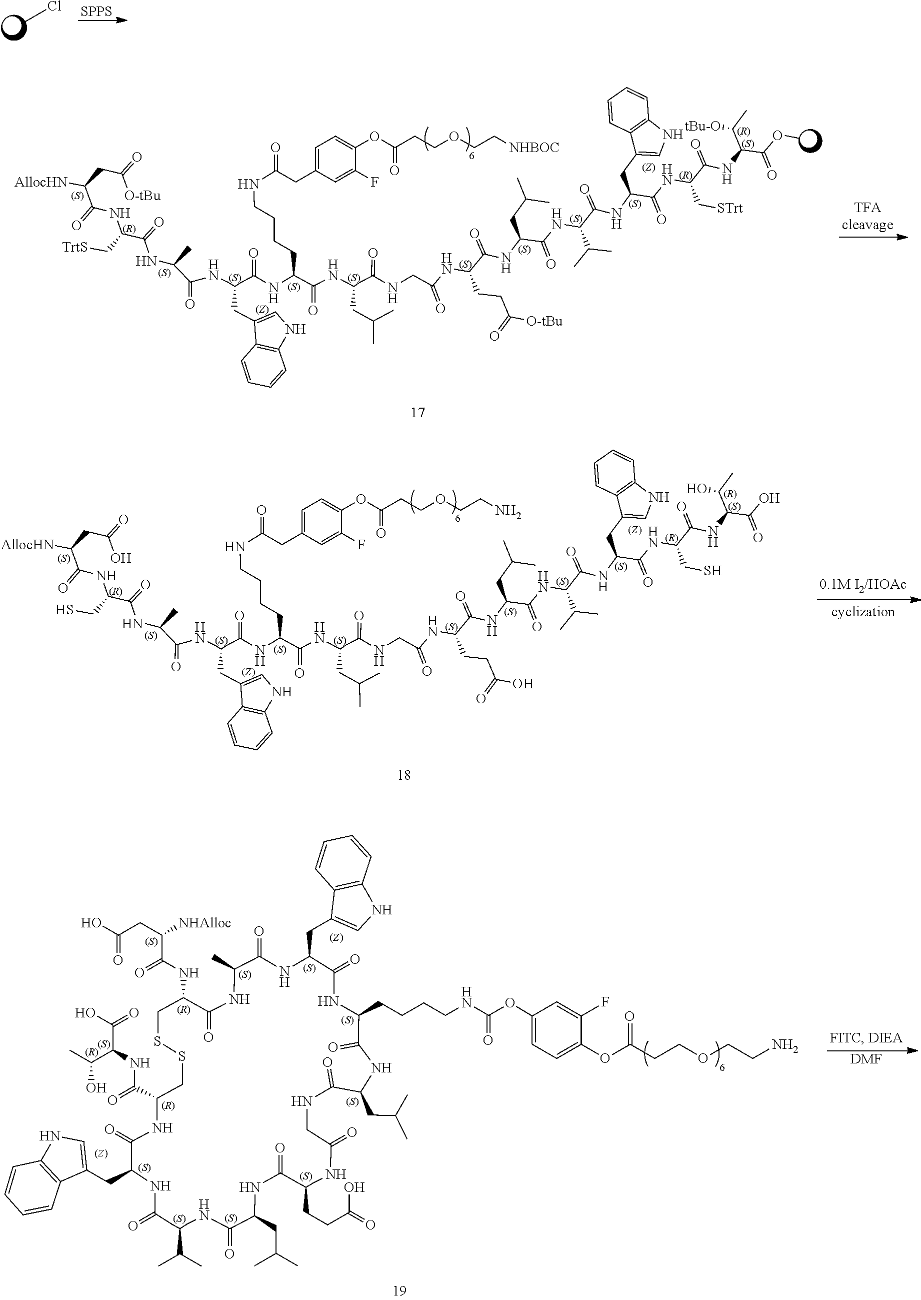

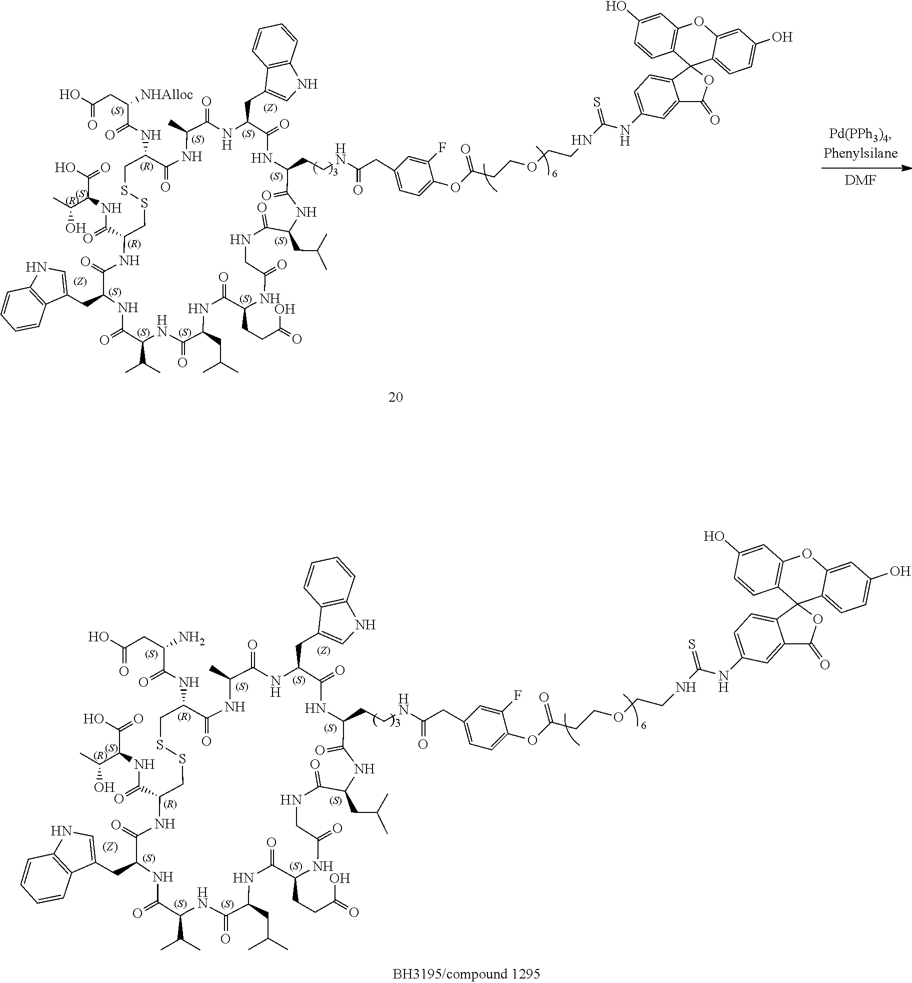

### Preparation of Intermediate 19:

Peptide was synthesized using standard Fmoc chemistry (CTC resin).

Resin preparation: To the vessel containing CTC resin (0.50 g, 0.50 mmol, 1.00 mmol/g) and Fmoc-Thr(tBu)-OH (198.5 mg, 0.50 mmol, 1.00 equiv.) in DCM (5 mL) was added DIEA (4.00 equiv.) dropwise and mixed for 2 h with N₂ bubbling at 25° C. Then MeOH (0.5 mL) was added and bubbled with N₂ for another 30 min. The resin was washed with DMF (10 mL), followed by the addition of 20% piperidine in DMF (10 mL) and bubbled with N₂ for 30 min at 25° C.for Fmoc deprotection. The mixture was filtered and the resin was washed with DMF (10 mL) before proceeding to next step.

Coupling: A solution of Fmoc-Cys(Trt)-OH (0.88 g, 1.5 mmol, 3.00 equiv.), HBTU (0.41 g, 1.43 mmol, 2.85 equiv.) in DMF (5 mL) was added to the resin with N2 bubbling. Then DIEA (6.00 equiv.) was added to the mixture dropwise and bubbled with N2 for 30 min at 25° C. The coupling reaction was monitored by ninhydrin test, if it showed colorless, the coupling was completed. The resin was then washed with DMF (20 mL).

Deprotection: 20% piperidine in DMF (10 mL) was added to the resin and the mixture was bubbled with N2 for 30 min at 25° C. The deprotection reaction was monitored by ninhydrin test, if it showed blue or brownish red, the reaction was completed. The resin was then washed with DMF (10 mL).

Steps 2 and 3 were repeated for the following amino acids elongation: Number # 3-9, Table 4.

Coupling: A solution of 2-(3-fluoro-4-hydroxyphenyl)acetic acid (253.5 mg, 1.50 mmol, 3.00 equiv.), HOBt (189.0 mg, 202.5 mg, 1.50 mmol, 3.00 equiv.) in DMF (5 mL) was added to the resin with N2 bubbling. Then DIC (1.50 mmol, 3.00 equiv.) was added to the mixture dropwise and bubbled with N2 for 30 min at 25° C. The coupling reaction was monitored by ninhydrin test, if it showed colorless, the coupling was completed. The resin was then washed with DMF (10 mL).

Dde deprotection: 3% Hydrazine hydrate in DMF (10 mL) was added to the resin with N2 bubbling for 30 min. Then the deprotection reaction was monitored by ninhydrin test, if it showed blue or brownish red, the reaction was completed. The resin was then washed with DMF (10 mL).

Coupling: A solution of Fmoc-Trp-OH (639.0 mg, 1.50 mmol, 3.00 equiv.), HOBt (202.5 mg, 1.50 mmol, 3.00 equiv.) in DMF (5 mL) was added to the resin with N₂ bubbling. Then DIC (1.50 mmol, 3.00 equiv.) was added to the mixture dropwise and bubbled with N₂ for 30 min at 25° C. The coupling reaction was monitored by ninhydrin test, if it showed colorless, the coupling was completed. The resin was then washed with DMF (10 mL).

Deprotection: 20% piperidine in DMF (10 mL) was added to the resin and the mixture was bubbled with N2 for 30 min at 25° C. The deprotection reaction was monitored by ninhydrin test, if it showed blue or brownish red, the reaction was completed. The resin was then washed with DMF (10 mL).

Steps 7 and 8 were repeated for the following amino acids elongation: Number # 11-14, Table 5.

Alloc-CI coupling on N-terminal: the resin was washed with DCM (20 mL). A solution of Allo-CI (0.36 g, 3.0 mmol, 6.00 equiv.) in DCM (10 mL) was added to the resin with N2 bubbling. Then DIEA (6.00 equiv.) was added to the mixture dropwise and bubbled with N2 for 30 min at 25° C. The coupling reaction was monitored by ninhydrin test, if it showed colorless, the coupling was completed. The resin was then washed with DCM (50 ml), DMF (50 mL).

Coupling: A solution of BocHN-PEG6-CH2CH2COOH (700.0 mg, 1.50 mmol, 3.00 equiv.), HOBt (202.5 mg, 1.50 mmol, 3.00 equiv.), DMAP (61.0 mg, 0.50 mmol, 1.00 equiv.) in DMF (5 mL) was added to the resin with N₂ bubbling. Then DIC (1.50 mmol, 3.00 equiv.) was added to the mixture dropwise and bubbled with N₂ for 16 h at 25° C. The coupling reaction was monitored by ninhydrin test, if it showed colorless, the coupling was completed. The resin was then washed with DMF (10 mL), MeOH (50 mL), then dried under reduced pressure to afford resin-bound peptide intermediate 17 (CTC resin, 1.30 g, 0.50 mmol)

TABLE 5
The list of amino acids and the corresponding reagents used on SPPS.

| # | Materials | Coupling reagents |
|---|---|---|
| 1 | Fmoc-Thr(tBu)-OH (1.00 equiv.) | DIEA (4.00 equiv.) |
| 2 | Fmoc-Cys(Trt)-OH (3.00 equiv.) | HBTU (2.85 equiv.) and |
| | | DIEA (6.00 equiv.) |
| 3 | Fmoc-Trp-OH (3.00 equiv.) | HBTU (2.85 equiv.) and |
| | | DIEA (6.00 equiv.) |
| 4 | Fmoc-Val-OH (3.00 equiv.) | HBTU (2.85 equiv.) and |
| | | DIEA (6.00 equiv.) |
| 5 | Fmoc-Leu-OH (3.00 equiv.) | HBTU (2.85 equiv.) and |
| | | DIEA (6.00 equiv.) |
| 6 | Fmoc-Glu(OtBu)-OH (3.00 equiv.) | HBTU (2.85 equiv.) and |
| | | DIEA (6.00 equiv.) |
| 7 | Fmoc-Gly-OH (3.00 equiv.) | HBTU (2.85 equiv.) and |
| | | DIEA (6.00 equiv.) |
| 8 | Fmoc-Leu-OH (3.00 equiv.) | HBTU (2.85 equiv.) and |
| | | DIEA (6.00 equiv.) |
| 9 | Dde-Lys(Fmoc)-OH (3.00 equiv.) | HBTU (2.85 equiv.) and |
| | | DIEA (6.00 equiv.) |
| 10 | 2-(3-fluoro-4-hydroxy- | DIC (3.00 equiv.) and |
| | phenyl)acetic acid (3.00 equiv.) | HOBt (3.00 equiv.) |
| 11 | Fmoc-Trp-OH (3.00 equiv.) | DIC (3.00 equiv.) and |
| | | HOBt (3.00 equiv.) |
| 12 | Fmoc-Ala-OH (3.00 equiv.) | DIC (3.00 equiv.) and |
| | | HOBt (3.00 equiv.) |
| 13 | Fmoc-Cys(Trt)-OH (3.00 equiv.) | DIC (3.00 equiv.) and |
| | | HOBt (3.00 equiv.) |
| 14 | Fmoc-Asp(OtBu)-OH (3.00 equiv.) | DIC (3.00 equiv.) and |
| | | HOBt (3.00 equiv.) |
| 15 | Alloc-Cl (6.00 equiv.) | DIEA (12.00 equiv.) |
| 16 | BocHN-PEG6-CH2CH2COOH | DIC (3.00 equiv.), |
| | (3.00 equiv.) | HOBt (3.00 equiv.), |
| | | DMAP (3.00 equiv.) |

Peptide Cleavage and Cyclization:

Cleavage: A solution of TFA/TIS/H2O/3-mercaptopropanoic acid (92.5/2.5/2.5/2.5, v/v/v, 30 ml) was added to the resin (intermediate 17, 0.50 mmol) at room temperature and stirred for 2 h. After filtration, After filtration, the filtrate was collected and precipitated with cold isopropyl ether (150 mL), then filtered off, and the solid was washed with isopropyl ether (100 mL) twice, and the crude peptide was dried under reduced pressure for 2 h to afford intermediate 18 (0.50 mmol, crude) as a white solid.

Cyclization: To the crude peptide (intermediate 18) in MeCN/H₂O (1/1, v/v, 500 mL) was added 0.1 M I₂/AcOH dropwise until a yellow color persisted, then the mixture was stirred at 25° C.for 5 min. The mixture was quenched by addition of 0.1 M aq. Na₂S₂O₃ dropwise until the yellow color disappeared. After filtration, the filtrate was purified by prep-HPLC (A: 0.075% TFA/H₂O, B: MeCN), followed by lyophilization to afford intermediate 19 (150.1 mg, 94.6% purity, 14.3% yield) as a white solid. LCMS: RT=1.00 min, MS calcd.: Mₐᵥ=2093.35, mass observed: [M+2H]²⁺=1048.2, [M+3H]³⁺=880.56.

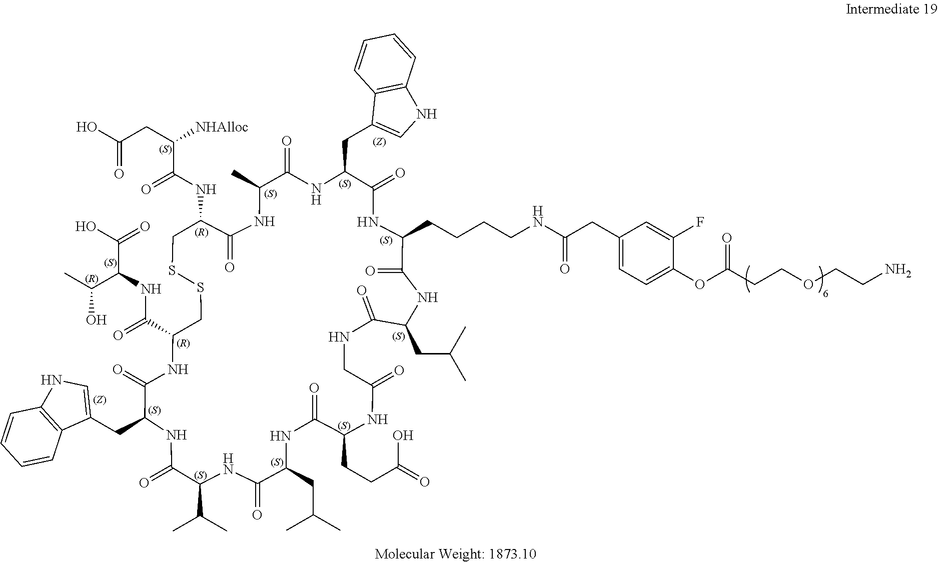

### Preparation of Intermediate 20:

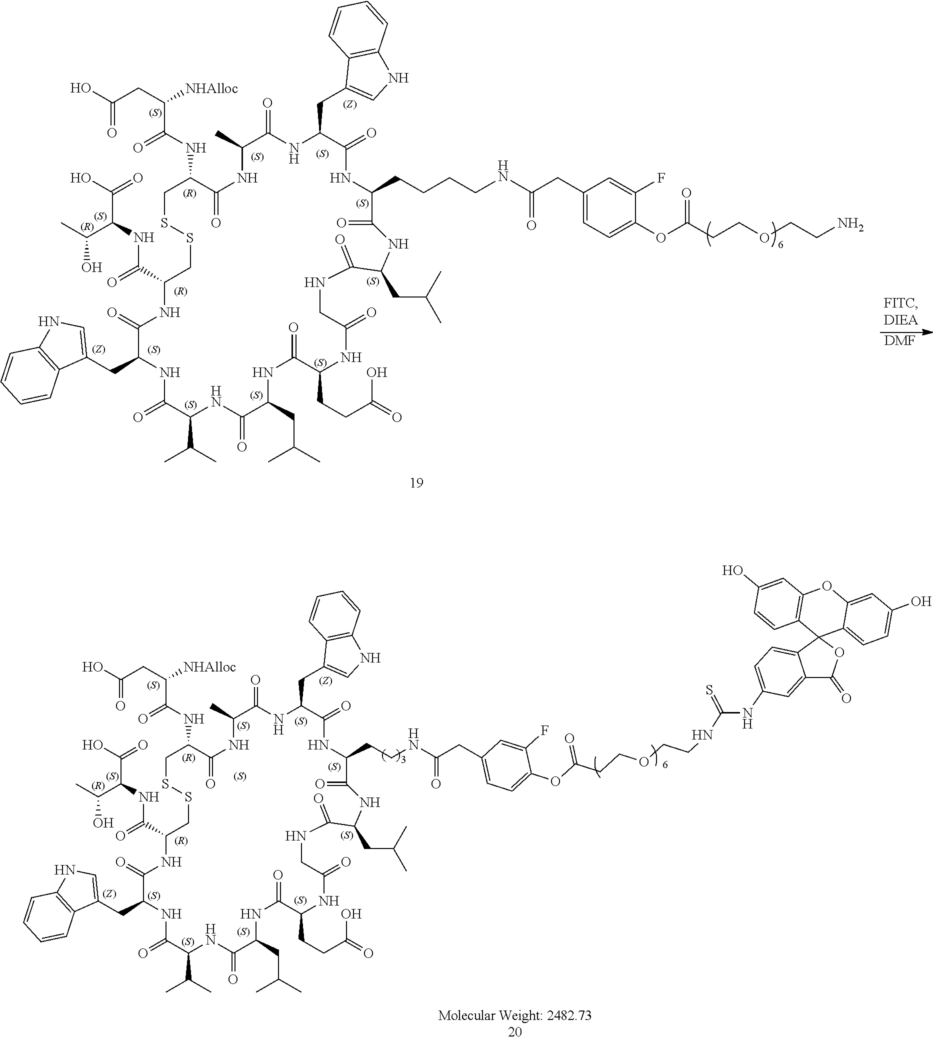

To a mixture of intermediate 19 (150.1 mg, 71.7 μmol, 1.00 equiv.) and FITC (41.9 mg, 107.55 μmol, 1.50 equiv.) in DMF (2 mL) was added DIEA (27.7 mg, 37.5 μL, 515.1 μmol, 3.00 equiv.) at 25° C. The mixture was stirred at 25° C.for 2 h. After completion monitored by LC-MS, the mixture was acidified by 1 M HCl to pH=5, then purified by prep-HPLC (A: 0.075% TFA/H₂O, B: MeCN) directly, followed by lyophilization to afford intermediate 20 (80.3 mg, 77.4% purity, 34.9% yield) as a yellow solid. LCMS: RT =1.06 min, MS calcd.: Mₐᵥ=2482.73, mass observed: [2M+3H]³⁺=1655.79, [M+2H]²⁺=1242.10, [M+3H]³⁺=828.31, [M+4H]⁴⁺=621.72.

### Preparation of Compound 1295:

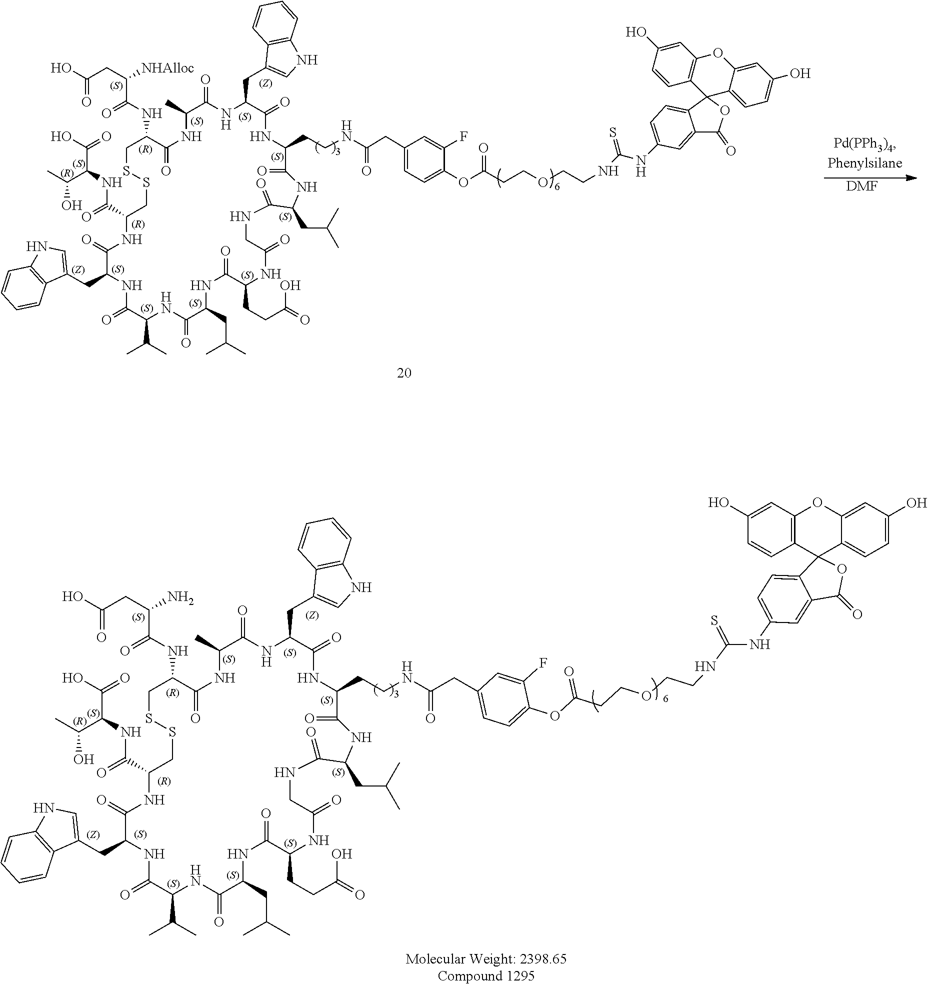

To a mixture of intermediate 20 (80.30 mg, 32.30 μmol, 1.00 equiv.) dissolved in DMF (1 mL) was added Pd(PPh₃)₄ (5.60 mg, 4.84 μmol, 0.15 equiv.) and phenylsilane (35.0 mg, 323.0 μmol, 10.00 equiv.). The mixture was stirred at 20° C. for 1 h. and the resulting reaction was stirred for 5 min at 0° C. After completion monitored by LC-MS. The mixture was acidified by 1 M HCl to pH=5, then purified by prep-HPLC (A: 0.075% TFA/H₂O, B: MeCN) directly, followed by lyophilization to afford compound 1295 (17.0 mg, 94.9% purity, 15.9% yield) as a yellow solid. LCMS: RT=2.02 min, MS calcd.: Mₐᵥ=2398.65, mass observed: [2M+3H]³⁺=1599.8, [M+H+Na]²⁺=1211.6, [M+2H]²⁺=1200.1, [M+3H]³⁺=800.4.

### Example 6. Procedure for Preparation of Compound 1293.

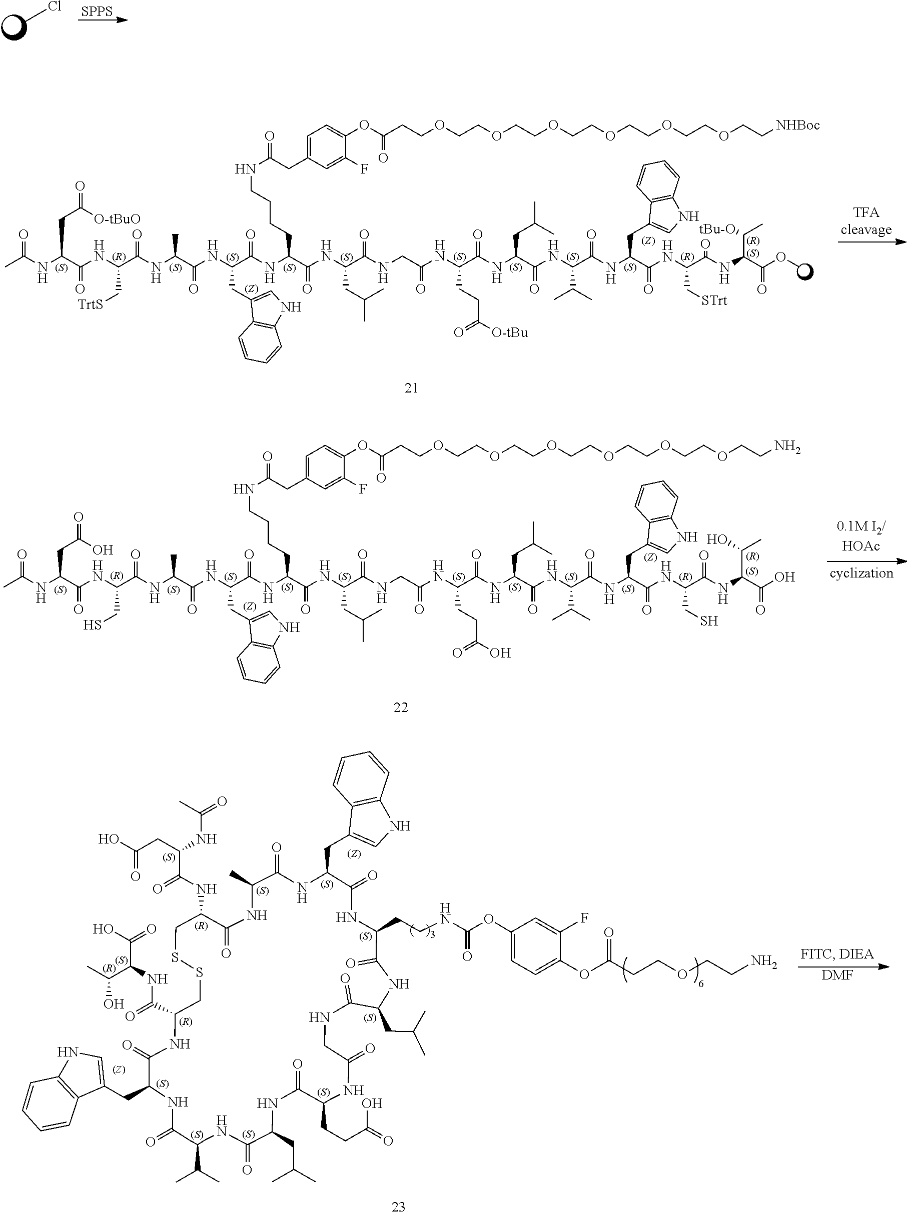

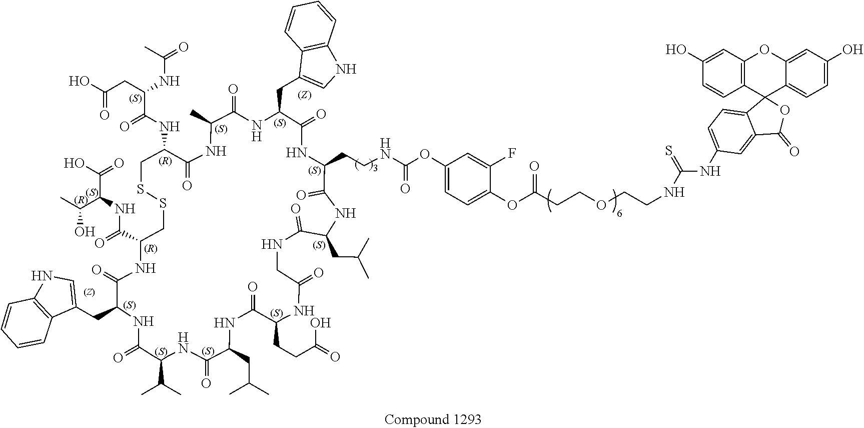

### Preparation of Intermediate 23:

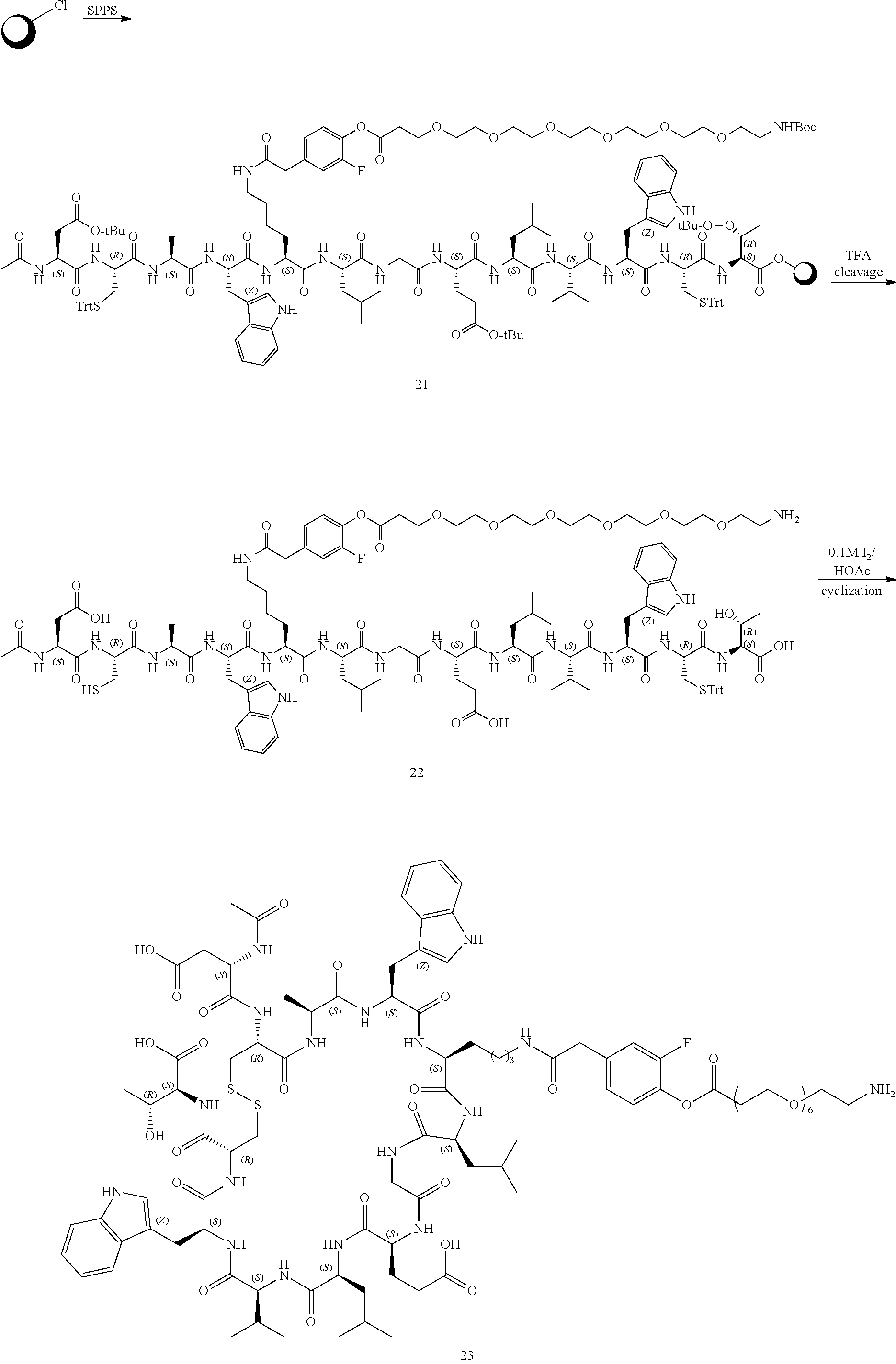

Peptide was synthesized using standard Fmoc chemistry (CTC resin).

Resin preparation: To the vessel containing CTC resin (0.50 g, 0.50 mmol, 1.00 mmol/g) and Fmoc-Thr(tBu)-OH (198.5 mg, 0.50 mmol, 1.00 equiv.) in DCM (5 mL) was added DIEA (4.00 equiv.) dropwise and mixed for 2 h with N₂ bubbling at 25° C. Then MeOH (0.5 mL) was added and bubbled with N₂ for another 30 min. The resin was washed with DMF (10 mL), followed by the addition of 20% piperidine in DMF (10 mL) and bubbled with N₂ for 30 min at 25° C.for Fmoc deprotection. The mixture was filtered and the resin was washed with DMF (10 mL) before proceeding to next step.

Coupling: A solution of Fmoc-Cys(Trt)-OH (0.88 g, 1.5 mmol, 3.00 equiv.), HBTU (0.41 g, 1.43 mmol, 2.85 equiv.) in DMF (5 mL) was added to the resin with N2 bubbling. Then DIEA (6.00 equiv.) was added to the mixture dropwise and bubbled with N₂ for 30 min at 25° C. The coupling reaction was monitored by ninhydrin test, if it showed colorless, the coupling was completed. The resin was then washed with DMF (20 mL).

Deprotection: 20% piperidine in DMF (10 mL) was added to the resin and the mixture was bubbled with N2 for 30 min at 25° C. The deprotection reaction was monitored by ninhydrin test, if it showed blue or brownish red, the reaction was completed. The resin was then washed with DMF (10 mL).

Steps 2 and 3 were repeated for the following amino acids elongation: Number # 3-9, Table 6.

Coupling: A solution of 2-(3-fluoro-4-hydroxyphenyl)acetic acid (253.5 mg, 1.50 mmol, 3.00 equiv.), HOBt (189.0 mg, 202.5 mg, 1.50 mmol, 3.00 equiv.) in DMF (5 mL) was added to the resin with N2 bubbling. Then DIC (1.50 mmol, 3.00 equiv.) was added to the mixture dropwise and bubbled with N2 for 30 min at 25° C. The coupling reaction was monitored by ninhydrin test, if it showed colorless, the coupling was completed. The resin was then washed with DMF (10 mL).

Dde deprotection: 3% Hydrazine hydrate in DMF (10 mL) was added to the resin with N2 bubbling for 30 min. Then the deprotection reaction was monitored by ninhydrin test, if it showed blue or brownish red, the reaction was completed. The resin was then washed with DMF (10 mL).

Coupling: A solution of Fmoc-Trp-OH (639.0 mg, 1.50 mmol, 3.00 equiv.), HOBt (202.5 mg, 1.50 mmol, 3.00 equiv.) in DMF (5 mL) was added to the resin with N₂ bubbling. Then DIC (1.50 mmol, 3.00 equiv.) was added to the mixture dropwise and bubbled with N₂ for 30 min at 25° C. The coupling reaction was monitored by ninhydrin test, if it showed colorless, the coupling was completed. The resin was then washed with DMF (10 mL).

Deprotection: 20% piperidine in DMF (10 mL) was added to the resin and the mixture was bubbled with N₂ for 30 min at 25° C. The deprotection reaction was monitored by ninhydrin test, if it showed blue or brownish red, the reaction was completed. The resin was then washed with DMF (10 mL).

Steps 7 and 8 were repeated for the following amino acids elongation: Number # 11-14, Table 6.

Acetylation: A solution of Ac2O/NMM/DMF (2/1/17, v/v/v, 40 mL) was added to the resin, the mixture was bubbled with N2 for 20 min. The acetylation reaction was monitored by ninhydrin test. The resin was then washed with DMF (20 mL).

Coupling: A solution of BocHN-PEG₆-CH₂CH₂COOH (700.0 mg, 1.50 mmol, 3.00 equiv.), HOBt (202.5 mg, 1.50 mmol, 3.00 equiv.), DMAP (61.0 mg, 0.50 mmol, 1.00 equiv.) in DMF (5 mL) was added to the resin with N₂ bubbling. Then DIC (1.50 mmol, 3.00 equiv.) was added to the mixture dropwise and bubbled with N₂ for 16 h at 25° C. The coupling reaction was monitored by ninhydrin test, if it showed colorless, the coupling was completed. The resin was then washed with DMF (10 mL), MeOH (50 mL), then dried under reduced pressure to afford resin-bound peptide intermediate 21 (CTC resin, 1.35 g, 0.50 mmol).

TABLE 6
The list of amino acids and the corresponding reagents used on SPPS.

| # | Materials | Coupling reagents |
|---|---|---|
| 1 | Fmoc-Thr(tBu)-OH (1.00 equiv.) | DIEA (4.00 equiv.) |
| 2 | Fmoc-Cys(Trt)-OH (3.00 equiv.) | HBTU (2.85 equiv.) and DIEA (6.00 equiv.) |
| 3 | Fmoc-Trp-OH (3.00 equiv.) | HBTU (2.85 equiv.) and DIEA (6.00 equiv.) |
| 4 | Fmoc-Val-OH (3.00 equiv.) | HBTU (2.85 equiv.) and DIEA (6.00 equiv.) |
| 5 | Fmoc-Leu-OH (3.00 equiv.) | HBTU (2.85 equiv.) and DIEA (6.00 equiv.) |
| 6 | Fmoc-Glu(OtBu)-OH (3.00 equiv.) | HBTU (2.85 equiv.) and DIEA (6.00 equiv.) |
| 7 | Fmoc-Gly-OH (3.00 equiv.) | HBTU (2.85 equiv.) and DIEA (6.00 equiv.) |
| 8 | Fmoc-Leu-OH (3.00 equiv.) | HBTU (2.85 equiv.) and DIEA (6.00 equiv.) |
| 9 | Dde-Lys(Fmoc)-OH (3.00 equiv.) | HBTU (2.85 equiv.) and DIEA (6.00 equiv.) |
| 10 | 2-(3-fluoro-4-hydroxyphenyl)acetic acid | DIC (3.00 equiv.) and HOBt (3.00 equiv.) |
| | (3.00 equiv.) | |
| 11 | Fmoc-Trp-OH (3.00 equiv.) | DIC (3.00 equiv.) and HOBt (3.00 equiv.) |
| 12 | Fmoc-Ala-OH (3.00 equiv.) | DIC (3.00 equiv.) and HOBt (3.00 equiv.) |
| 13 | Fmoc-Cys(Trt)-OH (3.00 equiv.) | DIC (3.00 equiv.) and HOBt (3.00 equiv.) |
| 14 | Fmoc-Asp(OtBu)-OH (3.00 equiv.) | DIC (3.00 equiv.) and HOBt (3.00 equiv.) |
| 15 | Acetylation (6.00 equiv.) | Ac2O/NMM/DMF (10/5/85, 10 mL) |
| 16 | BocHN-PEG6-CH2CH2COOH (3.00 equiv.) | DIC (3.00 equiv.), HOBt (3.00 equiv.), DMAP |
| | | (3.00 equiv.) |

### Peptide Cleavage and Cyclization:

Cleavage: A solution of TFA/TIS/H2O/3-mercaptopropanoic acid (92.5/2.5/2.5/2.5, v/v/v, 30 mL) was added to the resin (intermediate 21, 0.50 mmol) at room temperature and stirred for 2 h. After filtration, the filtrate was collected and precipitated with cold isopropyl ether (150 mL), then filtered off, and the solid was washed with isopropyl ether (100 ml) twice, and the crude peptide was dried under reduced pressure for 2 h to afford intermediate 22 (0.50 mmol, crude) as a white solid.

Cyclization: To the crude peptide (intermediate 22) in MeCN/H₂O (1/1, v/v, 500 mL) was added 0.1 M I₂/AcOH dropwise until a yellow color persisted, then the mixture was stirred at 25° C.for 5 min. The mixture was quenched by addition of 0.1 M aq. Na₂S₂O₃ dropwise until the yellow color disappeared. After filtration, the filtrate was purified by prep-HPLC (A: 0.075% TFA/H2O, B: MeCN), followed by lyophilization to afford intermediate 23 (160.1 mg, 90.0% purity, 15.6% yield) as a white solid. LCMS: RT =7.9 min, MS calcd .: Mav =2051.31, mass observed: [M +2H]2+=1026.60, [M +3H]3+=684.59.

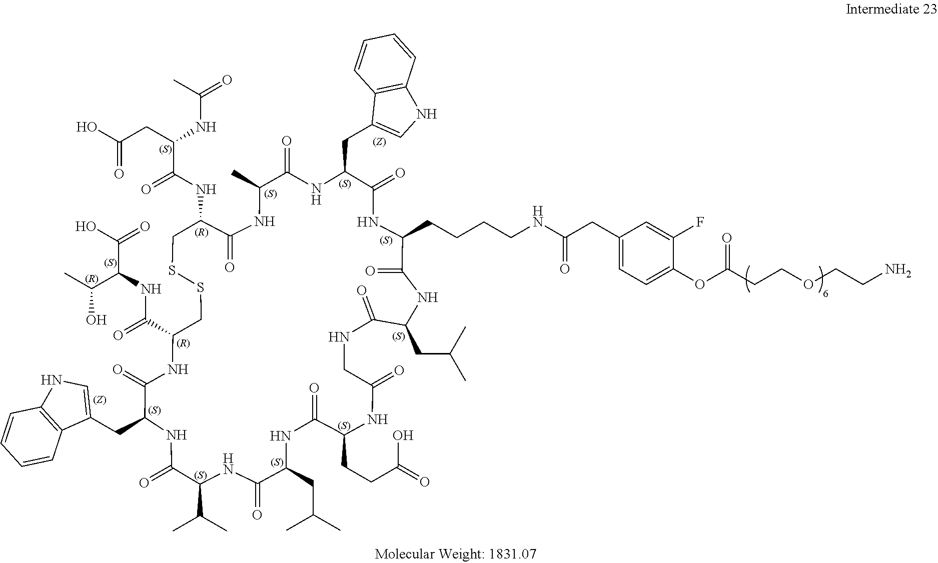

Preparation of Compound 1293:

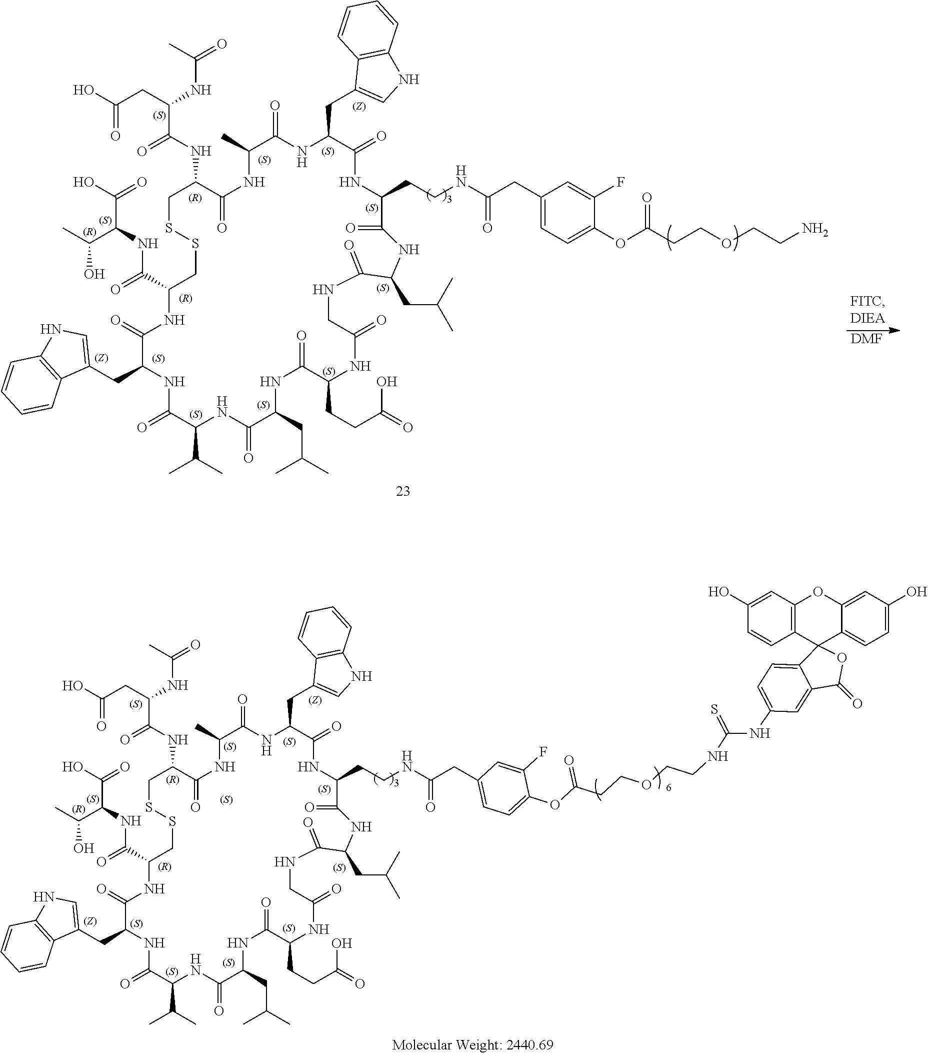

### Compound 1293

To a mixture of intermediate 23 (120.0 mg, 58.50 μmol, 1.00 equiv.) and FITC (34.08 mg, 87.51 μmol, 1.50 equiv.) in DMF (2 mL) was added DIEA (27.7 mg, 30.59 μL, 175.5 μmol, 3.00 equiv.) at 25° C. The mixture was stirred at 25° C.for 2 h. After completion monitored by LC-MS, the mixture was acidified by 1 M HCl to pH=5, then purified by prep-HPLC (A: 0.075% TFA/H₂O, B: MeCN) directly, followed by lyophilization to afford Compound 1293 (33.8 mg, 91.4% purity, 23.7% yield) as a yellow solid. LCMS: RT =2.02 min, MS calcd.: Mₐᵥ=2440.69, mass observed: [M+Na+H]²⁺=1232.00, [M+2H]²⁺=1221.60, [M +3H]³⁺=814.30.

We claim:

1. A compound having the structure of formula R-I: 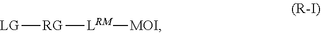
or a salt thereof, wherein:
LG is a group comprising a target binding moiety that binds to a target agent,
RG is a reactive group of formula -L^{LG2}-L^{LG3}-L^{LG4}-L^{RG1}-L^{RG2}-, wherein
L^{LG2} is —NH—C(O)O—C(R′)₂—, wherein each R′ is independently H or C1-C10 alkyl, wherein
R′ are optionally connected to form a ring;
L^{LG3} is an optionally substituted aryl ring;
L^{LG4-} is —NH— or —O—;
L^{RG1} is —C(O)—, —S(O)—, —OS(O)—, or —OP(O)(OR)₂—; and
L^{RG2}- is a covalent bond or [—C(R″)₂C(R″)═C(R″)]C(O)—, wherein each R″ is independently
H or C1-C10 alkyl, wherein any two R″ are optionally connected to form a ring;
L^{RM} is a linker; and
MOI is a moiety of interest.

2. The compound of claim 1, wherein RG is or comprises a reactive group having the following formula: 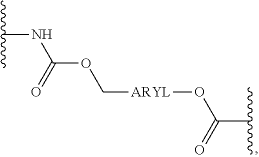
wherein ARYL is a substituted or unsubstituted para-phenylene ring.

3. The compound of claim 2, wherein ARYL has the structure of 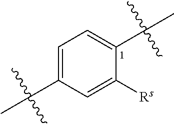
or 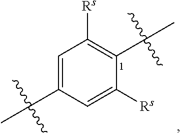
wherein R^{s} is independently chosen at each occurrence from halogen, —NO₂, —F, -L-R′, —C(O)-L-R′, —S(O)-L-R′, —S(O)₂-L-R′, and —P(O)(-L-R′)₂, and R′ is H or C₁-C₆alkyl.

4. The compound of claim 2 or 3, wherein the reactive group is or comprises has one of the following formulae:

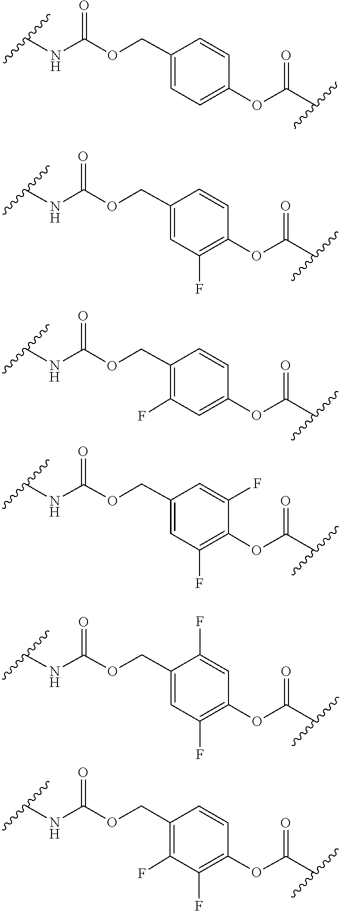

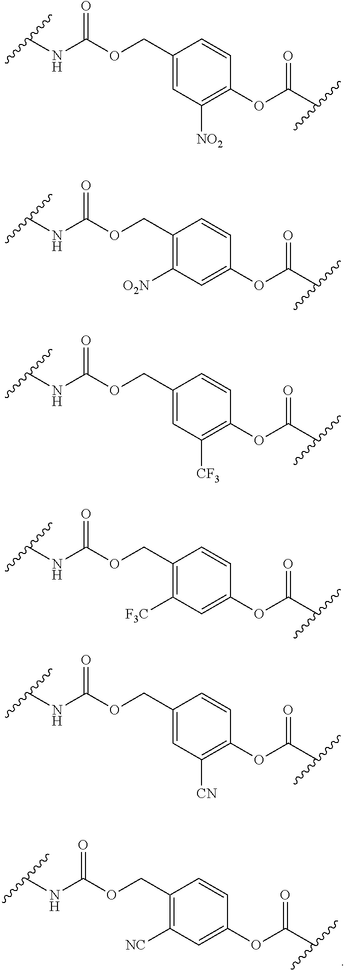 [SMILES: *NC(=O)OCc1ccc(OC(*)=O)c([N+](=O)[O-])c1 ; *NC(=O)OCc1ccc(OC(*)=O)cc1[N+](=O)[O-] ; *NC(=O)OCc1ccc(OC(*)=O)c(C(F)(F)F)c1 ; *NC(=O)OCc1ccc(OC(*)=O)cc1C(F)(F)F ; *NC(=O)OCc1ccc(OC(*)=O)c(C#N)c1 ; *NC(=O)OCc1ccc(OC(*)=O)cc1C#N]

5. The compound of claim 1, wherein RG is or comprises a reactive group having the following formula: 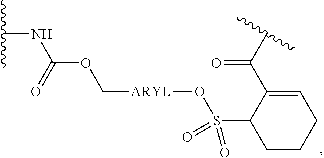
wherein ARYL is a substituted or unsubstituted para-phenylene ring.

6. The compound of claim 5, wherein ARYL has the structure of 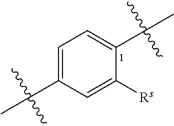
or 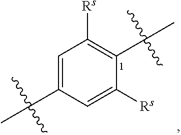
wherein R^{S} is independently chosen at each occurrence from halogen, —NO₂, —F, -L-R′, —C(O)-L-R′, —S(O)-L-R′, —S(O)₂-L-R′, and —P(O)(-L-R′)₂, and R′ is H or C₁-C₆alkyl.

7. The compound of claim 5 or 6, wherein the reactive group is or comprises has one of the following formulae:

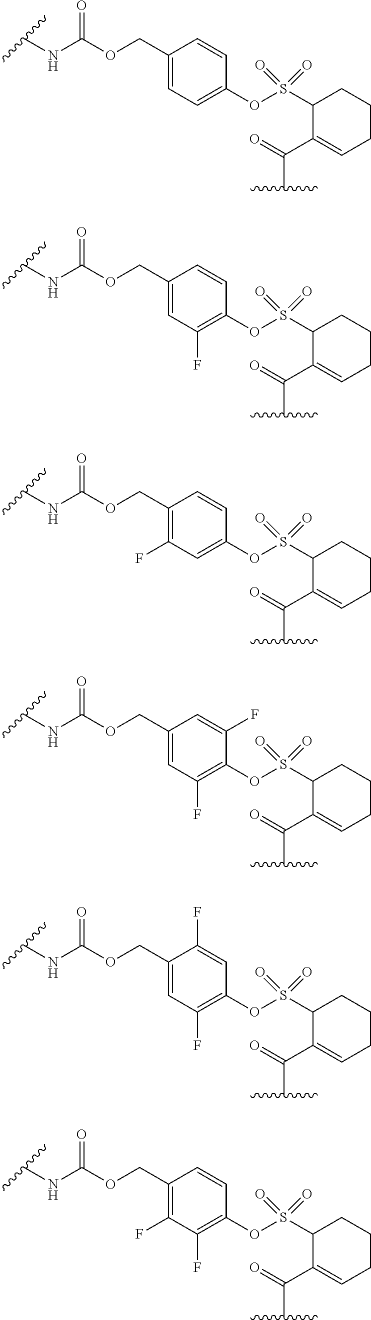 [SMILES: *NC(=O)OCc1ccc(OS(=O)(=O)C2CCCC=C2C(*)=O)cc1 ; *NC(=O)OCc1ccc(OS(=O)(=O)C2CCCC=C2C(*)=O)c(F)c1 ; *NC(=O)OCc1ccc(OS(=O)(=O)C2CCCC=C2C(*)=O)cc1F ; *NC(=O)OCc1cc(F)c(OS(=O)(=O)C2CCCC=C2C(*)=O)c(F)c1 ; *NC(=O)OCc1cc(F)c(OS(=O)(=O)C2CCCC=C2C(*)=O)cc1F ; *NC(=O)OCc1ccc(OS(=O)(=O)C2CCCC=C2C(*)=O)c(F)c1F]

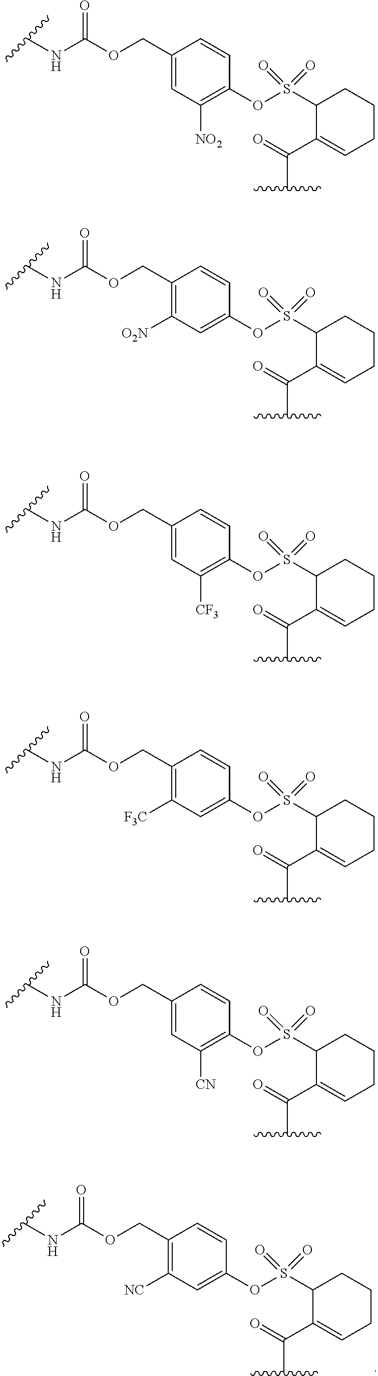 [SMILES: *NC(=O)OCc1ccc(OS(=O)(=O)C2CCCC=C2C(*)=O)c([N+](=O)[O-])c1 ; *NC(=O)OCc1ccc(OS(=O)(=O)C2CCCC=C2C(*)=O)cc1[N+](=O)[O-] ; *NC(=O)OCc1ccc(OS(=O)(=O)C2CCCC=C2C(*)=O)c(C(F)(F)F)c1 ; *NC(=O)OCc1ccc(OS(=O)(=O)C2CCCC=C2C(*)=O)cc1C(F)(F)F ; *NC(=O)OCc1ccc(OS(=O)(=O)C2CCCC=C2C(*)=O)c(C#N)c1 ; *NC(=O)OCc1ccc(OS(=O)(=O)C2CCCC=C2C(*)=O)cc1C#N]

8. The compound of claim 1, wherein
LG is R^{LG}-L^{LG};
R^{LG} is 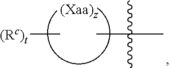
R^{c}—(Xaa)z-, a nucleic acid moiety, or a small molecule moiety;
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-;
each of L^{LG1}, L^{LG2}, L^{LG3}, and L^{LG4} is independently a covalent bond, or a bivalent optionally substituted, linear or branched C¹⁻¹⁰⁰ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—,—C(S)—, —C(NR')—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

9. The compound of claim 1, wherein LG is or comprises a target binding moiety that binds to a target agent, wherein the target agent is an antibody agent.

10. The compound of claim 1, wherein LG is or comprises a target binding moiety that binds to a Fc region, and/or R^{LG} is or comprises DCAWXLGELVWCT (SEQ ID NO:2), wherein the two cysteine residues optionally form a disulfide bond, and X is an amino acid residue.

11. The compound of claim 1, wherein at least one of the following conditions is met:
(a) the moiety of interest is or comprises a therapeutic agent;
(b) the moiety of interest is or comprises a moiety that can bind to a protein, nucleic acid or a cell; and/or
(c) the moiety of interest is or comprises a reactive moiety suitable for a bio-orthogonal reaction.

12. The compound of claim 1, wherein LG is or comprises a target binding moiety having the structure of formula A-1 to A-50 shown in the specification.

13. The compound of claim 1, wherein MOI is or comprises a therapeutic agent moiety and/or MOI is or comprises an antibody agent.

14. The compound of claim 1, wherein L^{RM} comprises one or more —[(CH₂)ₙ—O]ₘ—, wherein each n is independently 1-20, and m is 1-100.

15. The compound of claim 1, wherein in formula (R-I):
the target agent is an antibody comprising an IgG heavy chain comprising K246 or K248, and the target binding moiety is configured to bind the antibody so as to bring the reactive group in proximity with K246 or K248 of the IgG heavy chain to enable a reaction between K246 or K248 and the reactive group that results in attachment of a moiety comprising L^{RM}-MOI to K246 or K248 and expulsion of the group containing a target binding moiety from the compound.

16. A method of preparing an agent having the structure of P-I: 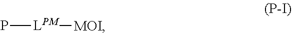
or a salt thereof, wherein:
P is a target agent moiety;
L^{PM} is a linker; and
MOI is a moiety of interest.
comprising steps of:
1) contacting a target agent with a reaction partner having the structure of formula R-I: 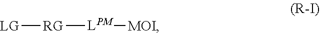
or a salt thereof, wherein:
LG is a group comprising a target binding moiety that binds to a target agent,
RG is a reactive group of formula -L^{LG2}-L^{LG3}-L^{LG4}-L^{RG1}-L^{RG2}-, wherein
L^{LG2} is —NH—C(O)O—C(R′)₂—, wherein each R′ is independently H or C1-C10 alkyl,
wherein R′ are optionally connected to form a ring;
L^{LG3} is an optionally substituted aryl ring;
L^{LG4-} is —NH— or —O—;
L^{RG1} is —C(O)—, —S(O)—, —OS(O)₂—, or —OP(O)(OR)₂—; and
L^{RG2}- is a covalent bond or [—C(R″)₂C(R″)═C(R″)]C(O)—, wherein each R″ is independently H or C1-C10 alkyl, wherein any two R″ are optionally connected to form a ring;
L^{RM} is a linker; and
MOI is a moiety of interest; and

2. forming an agent having the structure of formula P-I; or
a method of preparing an agent having the structure of P-II: 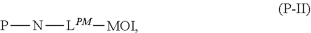
wherein:
P-N is a protein agent moiety comprising a lysine residue;
L^{PM} is a linker; and
MOI is a moiety of interest;
the method comprising:
contacting P-N with a reaction partner having a structure of formula R-I: 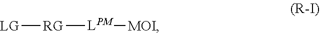
or a salt thereof, wherein:
LG is a group comprising a protein-binding moiety that binds to P-N,
RG is a reactive group of formula -L^{LG2}-L^{LG3}-L^{LG4}-L^{RG1}-L^{RG2}-, wherein
L^{LG2} is —NH—C(O)O—C(R′)₂-, wherein each R′ is independently H or C1-C10 alkyl, wherein R′ are optionally connected to form a ring;
L^{LG3} is an optionally substituted aryl ring;
L^{LG4-} is —NH— or —O—;
L^{RG1} is —C(O)—, —S(O)—, —OS(O)₂—, or —OP(O)(OR)₂—; and
L^{RG2}- is a covalent bond or [-C(R″)2C(R″)═C(R″)]C(O)—, wherein each R″ is independently H or C1-C10 alkyl, wherein any two R″ are optionally connected to form a ring;
L^{RM} is a linker; and
MOI is a moiety of interest.

17. The method of claim 16, wherein a target agent is or comprises an antibody agent.

18. The method of claim 17, wherein a moiety of interest is selectively attached to the antibody agent at K246 or K248 of an IgG1 heavy chain or a corresponding location.

19. The method of claim 17, wherein a moiety of interest is selectively attached to the antibody agent at K251 or K253 of an IgG2 heavy chain or a corresponding location.

20. The method of claim 17, wherein a moiety of interest is selectively attached to the antibody agent at K239 or K241 of an IgG4 heavy chain or a corresponding location.

21. The method of claim 16, wherein the contacting and forming steps are performed in one chemical reaction.

22. A composition comprising one or more compounds of any one of claims 1-15.

23. A composition comprising:
a first compound having the structure of formula (P-II): 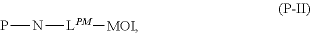
wherein:
P-N is a protein agent moiety comprising a lysine residue;
L^{PM} is a linker; and
MOI is a moiety of interest; and
a second compound having the structure: 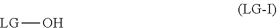
wherein LG is a group comprising a target binding moiety that binds to a target agent.

24. The composition of claim 23, further comprising:
a third compound having the formula (R-I): 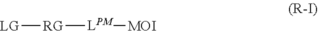
LG is a group comprising a target binding moiety that binds to a target agent, which is identical to LG in formula (LG-I);
RG is a reactive group of formula -L^{LG2}-L^{LG3}-L^{LG4}-L^{RG1}-L^{RG2}-, wherein
L^{LG2} is —NH—C(O)O—C(R′)₂—, wherein each R′ is independently H or C1-C10 alkyl, wherein R′ are optionally connected to form a ring;
L^{LG3} is an optionally substituted aryl ring;
L^{LG4-}is —NH— or —O—;
L^{RG1} is —C(O)—, —S(O)—, —OS(O)₂—, or —OP(O)(OR)2-; and
L^{RG2}- is a covalent bond or [—C(R″)₂C(R″)═C(R″)]C(O)—, wherein each R″ is independently H or C1-C10 alkyl, wherein any two R″ are optionally connected to form a ring;
L^{RM} is a linker, which is identical to in formula (P-II); and
MOI is a moiety of interest;
a fourth compound having the formula (R-III): 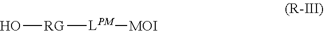
or a combination thereof.
